(19) United States
(12) Patent Application Publication
(10) Pub. No.: US 2024/0100081 A1
(43) Pub. Date: Mar. 28, 2024

(54) COMPOSITIONS AND METHODS FOR TREATING AGE-RELATED MACULAR DEGENERATION AND GEOGRAPHIC ATROPHY

(71) Applicant: University of Virginia Patent Foundation, Charlottesville, VA (US)

(72) Inventors: Jayakrishna Ambati, Charlottesville, VA (US); Nagaraj Kerur, Beaver Creek, OH (US)

(73) Assignee: University of Virginia Patent Foundation, Charlottesville, VA (US)

(21) Appl. No.: 18/316,981

(22) Filed: May 12, 2023

Related U.S. Application Data
(62) Division of application No. 16/754,565, filed on Apr. 8, 2020, now abandoned.
(60) Provisional application No. 62/570,207, filed on Oct. 10, 2017.

(51) Int. Cl. A61K 31/713; A61P 27/02; C07K 16/24; C12N 15/113; C12N 15/85

(52) U.S. Cl. CPC A61K 31/713; A61P 27/02; C07K 16/249; C12N 15/113; C12N 15/85; A61K 45/06

(57) ABSTRACT

It is disclosed herein that RPE degeneration in human cell culture and in mouse models is driven by a non-canonical inflammasome pathway that results in activation of caspase-4 (also known as caspase-11 in mouse) and caspase-1, and requires cyclic GMP-AMP synthase (cGAS)-dependent interferon-β (IFN-β) production and gasdermin D-dependent interleukin-18 (IL-18) secretion. Reduction of DICER1 or accumulation of Alu RNA triggers cytosolic escape of mitochondrial DNA, which engages cGAS. Collectively, these data highlight an unexpected role for cGAS in responding to mobile element transcripts, reveal cGAS-driven interferon signaling as a conduit for mitochondrial damage-induced NLRP3 activation, and expand the immune sensing repertoire of cGAS and caspase-4 to non-infectious human disease. Coupled with the unexpected result that caspase-4, gasdermin D, IFN-β, and cGAS are elevated in the RPE of human eyes with geographic atrophy, these findings also identify new targets for a major cause of blindness.

### CROSS REFERENCE TO RELATED APPLICATIONS

This application is a divisional of U.S. patent application Ser. No. 16/754,565, filed Apr. 8, 2020 (pending), which is a U.S. National Stage application of PCT International Patent Application Serial No. PCT/US2018/054941, filed Oct. 9, 2018, which itself claims priority under 35 U.S.C. § 119(e) to U.S. Provisional Application Ser. No. 62/570,207, filed Oct. 10, 2017. The disclosure of each of these applications is incorporated herein by reference in its entirety.

### GOVERNMENT INTEREST

This invention was made with government support under Grant Nos. GM114862, EY018350, EY018836, EY020672, EY022238, EY024068, and EY024336, awarded by The National Institutes of Health. The government has certain rights in the invention.

### REFERENCE TO SEQUENCE LISTING XML

The Sequence Listing XML associated with the instant disclosure has been electronically submitted to the United States Patent and Trademark Office via the Patent Center as a 25,837 byte UTF-8-encoded XML file created on Apr. 24, 2023 and entitled “3062_113_2_PCT_US_DIV.xml”. The Sequence Listing submitted via Patent Center is hereby incorporated by reference in its entirety.

### BACKGROUND

Age-related macular degeneration (AMD) affects over 180 million people⁴, and is increasingly the leading cause of blindness among the growing numbers of elderly across the world. Degeneration and death of the retinal pigmented epithelium (RPE), a monolayer of cells that provide trophic support to the photoreceptors⁵, is the hallmark of geographic atrophy and leads to vision loss. The RNase DICER1 is reduced in the RPE of human geographic atrophy eyes, leading to accumulation of toxic mobile element Alu RNA transcripts³; these Alu transcripts induce RPE cell death by activating the NLRP3 inflammasome². Although NLRP3 inflammasome activation has been widely implicated in macular degeneration⁶⁻¹⁰, the mechanisms regulating the inflammasome in this disease remain elusive.

There is a long felt need in the art for compositions and methods useful for treating diseases and disorders of the retinal pigmented epithelium such as age-related macular degeneration and geographic atrophy. The present invention satisfies these needs.

### SUMMARY OF THE INVENTION

The present application claims priority to a provisional application that was filed based on the draft of a manuscript by Kerur et al., which has since published in Nature Medicine as “cGAS drives non-canonical NLRP3 (NLR family pyrin domain containing 3) inflammasome in age-related macular degeneration” (Nat. Med. 2018, 24(1):50-61; available as epub. on Nov. 27, 2017).

It is disclosed herein that DICER1 deficit/Alu RNA-driven RPE degeneration in mouse models of macular degeneration is unexpectedly mediated by caspase-4- and gasdermin D-dependent NLRP3 inflammasome activation. Unexpectedly, it is also disclosed that this non-canonical inflammasome is dependent on the activation of the DNA sensor cyclic GMP-AMP synthase (cGAS)-driven type I interferon (IFN) signaling by cytosolic mitochondrial DNA (mtDNA).

Based on the unexpected results described above, further work disclosed herein demonstrates that RPE degeneration in macular degeneration can be inhibited or prevented by targeting the alternative, non-canonical inflammasome signaling molecules, protein complexes, or their signal transduction pathways. In one aspect, the molecules and pathways include, but are not limited to, cGAS, Caspase-4/11, Gasdermin D (GSDMD), stimulator of interferon genes (STING), peptidyl-prolyl cis-trans isomerase F (PPIF), mitochondrial permeability transition pore (MPTP), IFN-β, or interferon-α/β receptor (IFNAR). Administering an inhibitor or blocking the activity of Caspase-4/11, cGAS, GSDMD STING, PPIF, MPTP, IFN-β, or IFNAR, or their signal transduction pathways, can protect RPE cells from death, and be therapeutically useful for diseases such as geographic atrophy and age-related macular degeneration. In one aspect, the protein complex is MPTP (see FIG. 21 and Supplementary FIG. 15).

Data disclosed herein highlight an unexpected role for cGAS in responding to mobile element transcripts, reveal cGAS-driven interferon signaling as a conduit for mitochondrial damage-induced NLRP3 activation, and expand the immune sensing repertoire of cGAS and caspase-4 to non-infectious human disease. Coupled with the unexpected result that caspase-4, gasdermin D, IFN-β, and cGAS are elevated in the RPE of human eyes with geographic atrophy, these findings also identify new targets for a major cause of blindness.

In one embodiment, the present application discloses compositions and methods useful for inhibiting mitochondrial damage-induced NLRP3 activation.

Therefore, the present invention provides compositions and methods for protecting RPE cells against death or degeneration. In one aspect, the compositions and methods of the invention protect RPE cells by inhibiting one or more of the molecules of the non-canonical inflammasome signaling pathway as disclosed herein. In one aspect, the compositions and methods of the invention protect RPE cells by inhibiting one or more of Caspase-4 (a.k.a. Caspase-11 in mice), Gasdermin D, IFN-β, IFNAR, STING, cGAS, PPIF, or mitochondrial permeability transition pore (mPTP) opening. In one aspect, each is inhibited.

The present application demonstrates that, unexpectedly, cGAS is increased in geography atrophy RPE cells and that inhibiting cGAS can be useful for inhibiting Alu RNA-induced RPE cell death. The present application discloses that cGAS-driven interferon signaling is a conduit for mitochondrial-damage-induced inflammasome activation. In one embodiment, the application provides compositions and methods useful for inhibiting cGAS-driven signaling and inflammasome activation.

It is further disclosed herein that many signaling molecules can be targeted for preventing or slowing down RPE cell death in geographic atrophy and age-related macular degeneration (AMD). These signaling molecules have not been previously reported to play a role mediating RPE death. The present invention encompasses molecules, compositions, and methods useful to block the key signaling molecules including cGAS, Caspase-11/4, STING, MPTP, PPIF, Gasdermin D, IFNAR, or IFN-β. For example, it is disclosed herein that Gasdermin D is required for Alu RNA-induced RPE degeneration and inflammasome activation. In fact, it is disclosed herein for the first time that Gasdermin D is involved in a non-infectious human disease.

In one embodiment, the present invention provides compositions and methods for inhibiting Caspase-4 (Caspase-11). It is disclosed herein that Caspase-4 is required for Alu RNA-induced RPE degeneration and inflammasome activation.

In one embodiment, the compositions and methods of the invention are useful for inhibiting RPE cell death. In one aspect, the RPE cell death is Alu RNA-induced cell death. In one aspect, the compositions and methods of the invention are useful for inhibiting RPE cell death associated with age-related macular degeneration.

In one embodiment, the present invention provides compositions and methods for inhibiting GSDMD.

In one embodiment, the present invention provides compositions and methods for inhibiting STING.

In one embodiment, the present invention provides compositions and methods for inhibiting cGAS. In one aspect, inhibiting cGAS inhibits the cGAS-driven interferon signaling as disclosed herein. In one aspect, inhibiting cGAS-driven interferon signaling inhibits mitochondrial damage-induced NLRP3 activation.

In one embodiment, the present invention provides compositions and methods for inhibiting IFNAR.

In one embodiment, the present invention provides compositions and methods for inhibiting IFN-β.

In one embodiment, the present invention provides compositions and methods for inhibiting PPIF.

In one embodiment, the present invention provides compositions and methods for inhibiting mPTP.

The present application encompasses the use of multiple types of inhibitors. In one aspect, a useful inhibitor can be an antisense oligonucleotide, small interfering RNA (siRNA), short hairpin RNA (shRNA), antibody, and biologically active fragments or homologs of the antibody. In one aspect, a useful inhibitor of the invention is cGAS shRNA (shcGAS), cGAS siRNA, Caspase-4 shRNA, caspase-4 siRNA, or an IFN-β neutralizing antibody. In one aspect, a useful inhibitor of the invention is GSDMD shRNA, STING shRNA, PPIF shRNA, IFNB shRNA, or IFNAR1 shRNA.

Useful antibodies include monoclonal antibody, humanized antibody, chimeric antibody, single chain antibody, and biologically active fragments and homologs thereof.

In one embodiment, an inhibitor of INF-β is administered to a subject in need thereof. In one aspect, the inhibitor is an IFN-β neutralizing antibody or a biologically active fragment or homolog thereof.

In one embodiment, an inhibitor of cGAS is administered to a subject in need thereof. In one aspect, the inhibitor is an shRNA. In another aspect, it is an siRNA.

By “inhibiting” a molecule is meant that its expression, activity, or levels are decreased relative to what it would be in the diseased state or that it is blocked from increasing to what is found in the disease state.

A homolog of a protein or peptide (including antibodies) of the invention may comprise one or more conservative amino substitutions relative to the parent protein or peptide.

In one aspect, a homolog has sequence identity of about 70, 75, 80, 85, 90, 91, 92, 93, 94, 95, 96, 97, 98, or 99% with the parent. In one aspect, a homolog has sequence identity of at least about 70, 75, 80, 85, 90, 91, 92, 93, 94, 95, 96, 97, 98, or 99% with the parent molecule.

In one embodiment, a pharmaceutical composition comprising an effective amount of at least one therapeutic agent (inhibitor) of the invention is administered to a subject in need thereof. A subject in need thereof is one diagnosed with a disease or disorder such as age-related macular degeneration or geographic atrophy or is one who has been determined to be susceptible to a disease or disorder such as age-related macular degeneration or geographic atrophy. Methods and biomarkers are available for predicting whether a subject is susceptible to AMD, including, for example, genetic variants of complement factor H (CFH) and high-temperature requirement factor A-1 (HTRA1), smoking, and age.

In one aspect, at least two therapeutic agents or methods of the present invention are administered to a subject in need thereof. In one aspect, the at least two therapeutic agents are directed to one of the target molecules disclosed herein. In another aspect, a combination therapy is administered to target at least two different alternative, non-canonical inflammasome signaling molecules or protein complexes to prevent or inhibit RPE degeneration. When a subject has been diagnosed to be susceptible to an RPE disease or disorder, one or more of the therapeutic agents of the invention can be administered prophylactically. In one embodiment, administration of a therapeutic agent of the invention inhibits RPE degeneration.

In one aspect, the present application provides for treatment using at least one agent or method of the present invention regulating the non-canonical pathway in combination with other agents or methods known to be useful for treating or preventing Alu RNA induced RPE degeneration in AMD or geographic atrophy. Other agents and methods that are known include, but are not limited to, the use of agents to inhibit Alu RNA, stimulate DICER1, and inhibit IL-18, MyD88, the NLRP3 inflammasome, or Caspase 1. In one aspect, the inhibitor of Alu RNA is an siRNA or an antisense oligonucleotide.

The dose administered to a subject in need thereof can vary depending on the disease state as well as on the age, sex, weight, and health of the subject.

The model as presented in FIG. 21 (also referred to as Supplementary FIG. 15) summarizes the unexpected role of cGAS, Caspase 4/11, and Gasdermin D in RPE cell degeneration and also demonstrates the useful targets disclosed herein.

siRNA and shRNA targeting the cGAS, Caspase-4 and Gasdermin D are available and can also be made based on the known sequences of cGAS, Caspase-4, and Gasdermin D.

In one aspect, the human cGAS shRNA (TRCN0000146282) is 5′-CCGGCTTTGATAACTGCGTGACATACTCGAGTATGTCACGCAGTTATCAAAGTTTT TTG-3′ (SEQ ID NO: 1). In one aspect, the cGAS shRNA is SEQ ID NO: 15 (CCGGCCTGCTGTAACACTTCTTATTCTCGAGAATAAGAAGTGTTACAGCAGGTTT TTTG).

In one aspect, the human cGAS siRNA is human cGAS siRNA (SASI_Hs01_AAGAAGAAACATGGCGGCTATCCTTCTCTCACATCGAAAAGGAAATTT) (SEQ ID NO: 2).

In one aspect, an shRNA directed against Caspase-4 has SEQ ID NO: 16.

In one aspect, a human siRNA directed against Caspase-4 has a sequence selected from the group consisting of SEQ ID NOs: 9, 10, 11, 12, 13, and 14.

In one aspect, an shRNA directed against GSDMD has SEQ ID NO: 19.

In one aspect, an shRNA directed against STING has SEQ ID NO: 17.

In one aspect, an shRNA directed against PPIF has SEQ ID NO: 18.

In one aspect, an shRNA directed against IFNB has SEQ ID NO: 20.

In one aspect, an shRNA directed against IFNAR1 has SEQ ID NO: 21.

In one aspect, two or more different shRNAs are administered to a subject.

In one aspect, two or more different siRNAs are administered to a subject.

In one aspect, one shRNA and one siRNA are administered to a subject.

In one aspect, an inhibitory antibody, or a biologically active fragment or analog thereof, is administered to a subject in combination with an shRNA or an siRNA of the invention.

In one embodiment, the compositions and methods disclosed in the present application are useful for preventing or inhibiting blindness.

In one embodiment, additional therapeutic agents are administered in addition to the inhibitors of the invention. For example, additional therapeutic agent include, but are not limited to, cyclosporin A, Alu RNA antisense oligonucleotide, and a reverse transcriptase inhibitor.

Other agents can be administered, including antimicrobials.

The present invention further provides compositions and methods for method for inhibiting a non-canonical inflammasome signaling molecule, protein complex, or pathway in an RPE cell. In one aspect, the method comprises contacting an RPE cell with an effective amount of an inhibitor of at least one a non-canonical inflammasome molecule, protein complex, or pathway. In one aspect, the method comprises contacting the RPE cell with an inhibitor of at least one molecule or complex selected from the group consisting of cGAS, caspase-4, STING, PPIF, MPTP, GSDMD, IFN-β, and IFNAR. In one aspect, the type of inhibitor includes, but is not limited to, antisense oligonucleotide, small interfering RNA (siRNA), short hairpin RNA (shRNA), antibody, and biologically active fragments or homologs of the antibody. In one aspect, a homolog of an antibody or useful protein or peptide of the invention comprises at least 95% sequence identity with antibody, protein, or peptide. In one aspect, the antibody is a monoclonal antibody, humanized antibody, chimeric antibody, or single chain antibody. In one aspect, the useful inhibitors include, but are not limited to, shcGAS, cGAS siRNA, caspase-4 shRNA, caspase-4 siRNA and an IFN-β neutralizing antibody. In one aspect, shcGAS is SEQ ID NO: 1 or SEQ ID NO: 15 and cGAS siRNA is SEQ ID NO: 2. In one aspect, the inhibitor is Caspase-4 shRNA or Caspase-4 siRNA. In one aspect, the Caspase-4 shRNA is SEQ ID NO: 16. In one aspect, the Caspase-4 siRNA has a sequence selected from the group consisting of SEQ ID NOs: 9, 10, 11, 12, 13, and 14. In one aspect, the method protects said RPE cell from cell death. In one aspect, the method inhibits Alu RNA-induced RPE degeneration.

Also provided are compositions and methods useful for determining whether a subject has age-related macular degeneration or is susceptible to age-related macular degeneration. For example, the present application discloses an increase in each of Caspase-4, cGAS, and Gasdermin D to be associated with macular degeneration. One or more of these markers can be measured to determine whether a subject has macular degeneration. These methods can be used with known diagnostic methods previously in use for diagnosing macular degeneration, particularly age-related macular degeneration.

In one embodiment, the compositions and methods of the present application provide for treating a subject for age-related macular degeneration once the subject has been diagnosed with age-related macular degeneration.

In one embodiment, the levels of caspase-4, cGAS, or Gasdermin D, or at least two of the three, are determined in a subject and when the levels are found to be higher than control levels the subject is treated to inhibit RPE cell degeneration. In one aspect, it is suspected that the subject has or is developing age-related macular degeneration when one or more of the assays is performed. In one aspect, the determination of the levels of one or more of caspase-4, cGAS, or Gasdermin D is made as a routine preventative measure or checkup.

Kits are encompassed by the present invention and can include one of more of the therapeutic agents, optionally additional therapeutic compounds, an applicator, and an instructional material.

Various aspects and embodiments of the invention are described in further detail below.

Some useful sequences of the invention-

SEQ ID NO: 1:
human cGAS shRNA-
CCGGCTTTGATAACTGCGTGACATACTCGAGTATGTCACGCAGTTATCAA
AGTTTTTTG
SEQ ID NO: 2:
human cGAS siRNA-
AAGAAGAAACATGGCGGCTATCCTTCTCTCACATCGAAAAGGAAATTT
SEQ ID NO: 3:
human cGAS protein-
MQPWHGKAMQRASEAGATAPKASARNARGAPMDPTESPAAPEAALPKAG
KFGPARKSGSRQKKSAPDTQERPPVRATGARAKKAPQRAQDTQPSDATSA
PGAEGLEPPAAREPALSRAGSCRQRGARCSTKPRPPPGPWDVPSPGLPVS
APILVRRDAAPGASKLRAVLEKLKLSRDDISTAAGMVKGVVDHLLLRLKC
DSAFRGVGLLNTGSYYEHVKISAPNEFDVMFKLEVPRIQLEEYSNTRAYY
FVKFKRNPKENPLSQFLEGEILSASKMLSKFRKIIKEEINDIKDTDVIMK
RKRGGSPAVTLLISEKISVDITLALESKSSWPASTQEGLRIQNWLSAKVR
KQLRLKPFYLVPKHAKEGNGFQEETWRLSFSHIEKEILNNHGKSKTCCEN
KEEKCCRKDCLKLMKYLLEQLKERFKDKKHLDKFSSYHVKTAFFHVCTQN
PQDSQWDRKDLGLCFDNCVTYFLQCLRTEKLENYFIPEFNLFSSNLIDKR
SKEFLTKQIEYERNNEFPVFDEF
SEQ ID NO: 4:
human cGAS mRNA/nucleotide-
AGCCTGGGGTTCCCCTTCGGGTCGCAGACTCTTGTGTGCCCGCCAGTAGT
GCTTGGTTTCCAACAGCTGCTGCTGGCTCTTCCTCTTGCGGCCTTTTCCT
GAAACGGATTCTTCTTTCGGGGAACAGAAAGCGCCAGCCATGCAGCCTTG
GCACGGAAAGGCCATGCAGAGAGCTTCCGAGGCCGGAGCCACTGCCCCC
AAGGCTTCCGCACGGAATGCCAGGGGCGCCCCGATGGATCCCACCGAGT
CTCCGGCTGCCCCCGAGGCCGCCCTGCCTAAGGCGGGAAAGTTCGGCCC
CGCCAGGAAGTCGGGATCCCGGCAGAAAAAGAGCGCCCCGGACACCCA
GGAGAGGCCGCCCGTCCGCGCAACTGGGGCCCGCGCCAAAAAGGCCCCT
CAGCGCGCCCAGGACACGCAGCCGTCTGACGCCACCAGCGCCCCTGGGG
CAGAGGGGCTGGAGCCTCCTGCGGCTCGGGAGCCGGCTCTTTCCAGGGC
TGGTTCTTGCCGCCAGAGGGGCGCGCGCTGCTCCACGAAGCCAAGACCT
CCGCCCGGGCCCTGGGACGTGCCCAGCCCCGGCCTGCCGGTCTCGGCCC
CCATTCTCGTACGGAGGGATGCGGCGCCTGGGGCCTCGAAGCTCCGGGC
GGTTTTGGAGAAGTTGAAGCTCAGCCGCGATGATATCTCCACGGCGGCG
GGGATGGTGAAAGGGGTTGTGGACCACCTGCTGCTCAGACTGAAGTGCG
ACTCCGCGTTCAGAGGCGTCGGGCTGCTGAACACCGGGAGCTACTATGA
GCACGTGAAGATTTCTGCACCTAATGAATTTGATGTCATGTTTAAACTGG
AAGTCCCCAGAATTCAACTAGAAGAATATTCCAACACTCGTGCATATTA
CTTTGTGAAATTTAAAAGAAATCCGAAAGAAAATCCTCTGAGTCAGTTTT
TAGAAGGTGAAATATTATCAGCTTCTAAGATGCTGTCAAAGTTTAGGAA
AATCATTAAGGAAGAAATTAACGACATTAAAGATACAGATGTCATCATG
AAGAGGAAAAGAGGAGGGAGCCCTGCTGTAACACTTCTTATTAGTGAAA
AAATATCTGTGGATATAACCCTGGCTTTGGAATCAAAAAGTAGCTGGCC
TGCTAGCACCCAAGAAGGCCTGCGCATTCAAAACTGGCTTTCAGCAAAA
GTTAGGAAGCAACTACGACTAAAGCCATTTTACCTTGTACCCAAGCATG
CAAAGGAAGGAAATGGTTTCCAAGAAGAAACATGGCGGCTATCCTTCTC
TCACATCGAAAAGGAAATTTTGAACAATCATGGAAAATCTAAAACGTGC
TGTGAAAACAAAGAAGAGAAATGTTGCAGGAAAGATTGTTTAAAACTAA
TGAAATACCTTTTAGAACAGCTGAAAGAAAGGTTTAAAGACAAAAAACA
TCTGGATAAATTCTCTTCTTATCATGTGAAAACTGCCTTCTTTCACGTAT
GTACCCAGAACCCTCAAGACAGTCAGTGGGACCGCAAAGACCTGGGCCTC
TGCTTTGATAACTGCGTGACATACTTTCTTCAGTGCCTCAGGACAGAAAA
ACTTGAGAATTATTTTATTCCTGAATTCAATCTATTCTCTAGCAACTTAA
TTGACAAAAGAAGTAAGGAATTTCTGACAAAGCAAATTGAATATGAAAG
AAACAATGAGTTTCCAGTTTTTGATGAATTTTGAGATTGTATTTTTAGAA
AGATCTAAGAACTAGAGTCACCCTAAATCCTGGAGAATACAAGAAAAAT
TTGAAAAGGGGCCAGACGCTGTGGCTCAC
SEQ ID NO: 5:
human Caspase-4 protein-
MAEGNHRKKPLKVLESLGKDFLTGVLDNLVEQNVLNWKEEEKKKYYDAK
TEDKVRVMADSMQEKQRMAGQMLLQTFFNIDQISPNKKAHPNMEAGPPES
GESTDALKLCPHEEFLRLCKERAEEIYPIKERNNRTRLALIICNTEEDHL
PPRNGADFDITGMKELLEGLDYSVDVEENLTARDMESALRAFATRPEHKS
SDSTFLVLMSHGILEGICGTVHDEKKPDVLLYDTIFQIFNNRNCLSLKDK
PKVIIVQACRGANRGELWVRDSPASLEVASSQSSENLEEDAVYKTHVEKD
FIAFCSSTPHNVSWRDSTMGSIFITQLITCFQKYSWCCHLEEVFRKVQQS
FETPRAKAQMPTIERLSMTRYFYLFPGN
SEQ ID NO: 6:
human Caspase-4 mRNA/nucleotide-
ATACATAGTTTACTTTCATTTTTGACTCTGAGGCTCTTTCCAACGCTGTA
AAAAAGGACAGAGGCTGTTCCCTATGGCAGAAGGCAACCACAGAAAAAA
GCCACTTAAGGTGTTGGAATCCCTGGGCAAAGATTTCCTCACTGGTGTTT
TGGATAACTTGGTGGAACAAAATGTACTGAACTGGAAGGAAGAGGAAA
AAAAGAAATATTACGATGCTAAAACTGAAGACAAAGTTCGGGTCATGGC
AGACTCTATGCAAGAGAAGCAACGTATGGCAGGACAAATGCTTCTTCAA
ACCTTTTTTAACATAGACCAAATATCCCCCAATAAAAAAGCTCATCCGAA
TATGGAGGCTGGACCACCTGAGTCAGGAGAATCTACAGATGCCCTCAAG
CTTTGTCCTCATGAAGAATTCCTGAGACTATGTAAAGAAAGAGCTGAAG
AGATCTATCCAATAAAGGAGAGAAACAACCGCACACGCCTGGCTCTCAT
CATATGCAATACAGAGTTTGACCATCTGCCTCCGAGGAATGGAGCTGAC
TTTGACATCACAGGGATGAAGGAGCTACTTGAGGGTCTGGACTATAGTG
TAGATGTAGAAGAGAATCTGACAGCCAGGGATATGGAGTCAGCGCTGAG
GGCATTTGCTACCAGACCAGAGCACAAGTCCTCTGACAGCACATTCTTG
GTACTCATGTCTCATGGCATCCTGGAGGGAATCTGCGGAACTGTGCATG
ATGAGAAAAAACCAGATGTGCTGCTTTATGACACCATCTTCCAGATATTC
AACAACCGCAACTGCCTCAGTCTGAAGGACAAACCCAAGGTCATCATTG
TCCAGGCCTGCAGAGGTGCAAACCGTGGGGAACTGTGGGTCAGAGACTC
TCCAGCATCCTTGGAAGTGGCCTCTTCACAGTCATCTGAGAACCTAGAGG
AAGATGCTGTTTACAAGACCCACGTGGAGAAGGACTTCATTGCTTTCTGC
TCTTCAACGCCACACAACGTGTCCTGGAGAGACAGCACAATGGGCTCTA
TCTTCATCACACAACTCATCACATGCTTCCAGAAATATTCTTGGTGCTGC
CACCTAGAGGAAGTATTTCGGAAGGTACAGCAATCATTTGAAACTCCAA
GGGCCAAAGCTCAAATGCCCACCATAGAACGACTGTCCATGACAAGATA
TTTCTACCTCTTTCCTGGCAATTGAAAATGGAAGCCACAAGCAGCCCAGC
CCTCCTTAATCAACTTCAAGGAGCACCTTCATTAGTACAGCTTGCATATT
TAACATTTTGTATTTCAATAAAAGTGAAGACAAACGA
SEQ ID NO: 7:
human Gasdermin D protein-
MGSAFERVVRRVVQELDHGGEFIPVTSLQSSTGFQPYCLVVRKPSSSWFW
KPRYKCVNLSIKDILEPDAAEPDVQRGRSFHFYDAMDGQIQGSVELAAPG
QAKIAGGAAVSDSSSTSMNVYSLSVDPNTWQTLLHERHLRQPEHKVLQQL
RSRGDNVYVVTEVLQTQKEVEVTRTHKREGSGRFSLPGATCLQGEGQGHL
SQKKTVTIPSGSTLAFRVAQLVIDSDLDVLLFPDKKQRTFQPPATGHKRS
TSEGAWPQLPSGLSMMRCLHNFLTDGVPAEGAFTEDFQGLRAEVETISKE
LELLDRELCQLLLEGLEGVLRDQLALRALEEALEQGQSLGPVEPLDGPAG
AVLECLVLSSGMLVPELAIPVVYLLGALTMLSETQHKLLAEALESQTLLG
PLELVGSLLEQSAPWQERSTMSLPPGLLGNSWGEGAPAWVLLDECGLELG
EDTPHVCWEPQAQGRMCALYASLALLSGLSQEPH
SEQ ID NO: 8:
human Gasdermin D mRNA/nucleotide-
CCTGGGCGGGCCCTGCGTCAGGTTGCAGTTTCACTTTTAGCTCTGGGCAC
CTCCAGCTCCTGCTCGCCGGACGGCTCCCAGGGAGAGCAGACGCGCCAG
ACGCGCCACCCTCGGGGCGCCGACGGTCACGGAGCATGGGGTCGGCCTT
TGAGCGGGTAGTCCGGAGAGTGGTCCAGGAGCTGGACCATGGTGGGGA
GTTCATCCCTGTGACCAGCCTGCAGAGCTCCACTGGCTTCCAGCCCTACT
GCCTGGTGGTTAGGAAGCCCTCAAGCTCATGGTTCTGGAAACCCCGTTAT
AAGTGTGTCAACCTGTCTATCAAGGACATCCTGGAGCCGGATGCCGCGG
AACCAGACGTGCAGCGTGGCAGGAGCTTCCACTTCTACGATGCCATGGA
TGGGCAGATACAGGGCAGCGTGGAGCTGGCAGCCCCAGGACAGGCAAA
GATCGCAGGCGGGGCCGCGGTGTCTGACAGCTCCAGCACCTCAATGAAT
GTGTACTCGCTGAGTGTGGACCCTAACACCTGGCAGACTCTGCTCCATGA
GAGGCACCTGCGGCAGCCAGAACACAAAGTCCTGCAGCAGCTGCGCAGC
CGCGGGGACAACGTGTACGTGGTGACTGAGGTGCTACAGACACAGAAG
GAGGTGGAAGTCACGCGCACCCACAAGCGGGAGGGCTCGGGCCGGTTTT
CCCTGCCCGGAGCCACGTGCTTGCAGGGTGAGGGCCAGGGCCATCTGAG
CCAGAAGAAGACGGTCACCATCCCCTCAGGCAGCACCCTCGCATTCCGG
GTGGCCCAGCTGGTTATTGACTCTGACTTGGACGTCCTTCTCTTCCCGGA
TAAGAAGCAGAGGACCTTCCAGCCACCCGCGACAGGCCACAAGCGTTCC
ACGAGCGAAGGCGCCTGGCCACAGCTGCCCTCTGGCCTCTCCATGATGA
GGTGCCTCCACAACTTCCTGACAGATGGGGTCCCTGCGGAGGGGGCGTT
CACTGAAGACTTCCAGGGCCTACGGGCAGAGGTGGAGACCATCTCCAAG
GAACTGGAGCTTTTGGACAGAGAGCTGTGCCAGCTGCTGCTGGAGGGCC
TGGAGGGGGTGCTGCGGGACCAGCTGGCCCTGCGAGCCTTGGAGGAGGC
GCTGGAGCAGGGCCAGAGCCTTGGGCCGGTGGAGCCCCTGGACGGTCCA
GCAGGTGCTGTCCTGGAGTGCCTGGTGTTGTCCTCCGGAATGCTGGTGCC
GGAACTCGCTATCCCTGTTGTCTACCTGCTGGGGGCACTGACCATGCTGA
GTGAAACGCAGCACAAGCTGCTGGCGGAGGCGCTGGAGTCGCAGACCCT
GTTGGGGCCGCTCGAGCTGGTGGGCAGCCTCTTGGAGCAGAGTGCCCCG
TGGCAGGAGCGCAGCACCATGTCCCTGCCCCCCGGGCTCCTGGGGAACA
GCTGGGGCGAAGGAGCACCGGCCTGGGTCTTGCTGGACGAGTGTGGCCT
AGAGCTGGGGGAGGACACTCCCCACGTGTGCTGGGAGCCGCAGGCCCAG
GGCCGCATGTGTGCACTCTACGCCTCCCTGGCACTGCTATCAGGACTGAG
CCAGGAGCCCCACTAGCCTGTGCCCGGGCATGGCCTGGCAGCTCTCCAG
CAGGGCAGAGTGTTTGCCCACCAGCTGCTAGCCCTAGGAAGGCCAGGAG
CCCAGTAGCCATGTGGCCAGTCTACCATGGGGCCCAGGAGTTGGGGAAA
CACAATAAAGGTGGCATACGAAGGAAAAAAAAAAAAAAAAAAAAACCA
AAAAAAAAAAAAAAAAAAAAAAAAAAAAAAAAAAAAAAAAAAAAAAA
AAAAAAAA
SEQ ID NO: 9:
siRNA directed against Caspase-4, S1
GUGUAGAUGUAGAAGAGAATT
SEQ ID NO: 10:
siRNA directed against Caspase-4, S2
CCUAGAGGAAGAUGCUGUUTT
SEQ ID NO: 11:
siRNA directed against Caspase-4
CUACACUGUGGUUGACGAA
SEQ ID NO: 12:
siRNA directed against Caspase-4
CCAUAGAACGAGCAACCUU
SEQ ID NO: 13:
siRNA directed against Caspase-4
CAGCAGAAUCUACAAAUAU
SEQ ID NO: 14:
siRNA directed against Caspase-4
CGGAUGUGCUGCUUUAUGA
SEQ ID NO: 15:
shRNA against cGAS
CCGGCCTGCTGTAACACTTCTTATTCTCGAGAATAAGAAGTGTTACAG
CAGGTTTTTTG
SEQ ID NO: 16:
shRNA against Caspase-4
CCGGAGACTATGTAAAGAAAGAGCTCTCGAGAGCTCTTTCTTTACATA
GTCTTTTTT
SEQ ID NO: 17:
shRNA against STING
CCGGCCAACATTCGCTTCCTGGATACTCGAGTATCCAGGAAGCGAATG
TTGGTTTTTTG
SEQ ID NO: 18:
shRNA against PPIF
CCGGCTGTGGCCAGTTGAGCTAATCCTCGAGGATTAGCTCAACTGGCC
ACAGTTTTTG
SEQ ID NO: 19:
shRNA against GSDMD
CCGGCAACCTGTCTATCAAGGACATCTCGAGATGTCCTTGATAGACAG
GTTGTTTTTTG
SEQ ID NO: 20:
shRNA against IFNB
CCGGCAGAGTGGAAATCCTAAGGAACTCGAGTTCCTTAGGATTTCCAC
TCTGTTTTT
SEQ ID NO: 21:
shRNA against IFNAR1
CCGGGCCAAGATTCAGGAAATTATTCTCGAGAATAATTTCCTGAATCT
TGGCTTTTTG

The present application provides for the preparation and use of homologs and fragments of the sequences disclosed herein where the homologs and fragments have similar activity to the parent as disclosed herein.

siRNAs and shRNAs encompassed by the invention can be prepared based on the nucleic acid sequences provided above and herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1I. Caspase-4/11 in geographic atrophy and RPE degeneration. (FIG. 1A) Immunoblots show increased caspase-4 activation (p30 subunits) in the RPE of human eyes with geographic atrophy compared to unaffected controls (Ctr). Specific bands of interest are indicated by an arrow head. Bar graph shows densitometry of the bands corresponding to caspase-4 p30 normalized to loading control. (FIG. 1B) Activation of caspase-4 (p30 subunit) in human RPE cells by Alu RNA, pAlu, or DICER1 anti-sense oligonucleotides (DICER1 AS). Specific bands of interest are indicated by arrowheads. (FIG. 1C) Immunoblot shows subretinal injection of Alu RNA in WT mice induces activation of caspase-11 in the RPE. n=3. (FIG. 1D) Alu RNA induces RPE degeneration in WT but not Casp11^{-/-} mice. n=8-10. (FIG. 1E) Casp11^{-/-} mice expressing human caspase-4 transgene (Casp11^{-/-} hCasp4^{Tg}) are susceptible to Alu RNA-induced RPE degeneration. n=8. In fundus photographs (upper row), the degenerated retinal area is outlined by blue arrowheads. RPE cellular boundaries are visualized by immunostaining with zonula occludens-1 (ZO-1) antibody. Loss of regular hexagonal cellular boundaries represents degenerated RPE. (FIG. 1F) Alu RNA induced caspase-1 activation (p20 subunit) in the RPE of WT but not Casp11^{-/-} mice, n=3. (FIG. 1G) Alu RNA activates caspase-1 (p20 subunit) in WT but not in Casp11^{-/-} mouse RPE cells. (FIG. 1H) Induction of IL-18 secretion by Alu RNA in mouse WT and Casp11^{-/-} RPE cells. n=3; *P<0.05. Error bars denote SD. (FIG. 1I) Caspase-1 and caspase-11 deficient mice (Casp1^{-/-} Casp11^{129mt/129mt}) as well as Casp1^{-/-} Casp11^{129mt/129mt} mice expressing functional mouse caspase-11 from bacterial artificial chromosome transgene (Casp1^{-/-} Casp11^{129mt/129mt} Casp11^{Tg}) were not susceptible to Alu-induced RPE degeneration. n=7-8. Representative immunoblots of three independent experiments and densitometric analysis (mean (SEM)) are shown. Binary and morphometric quantification of RPE degeneration are shown (*, P<0.05; **, P<0.01; ***, P<0.001). PM, polymegethism (mean (SEM)).

FIGS. 2A-2L. Gasdermin D in geographic atrophy and RPE degeneration. (FIG. 2A) Alu RNA induced RPE degeneration in WT but not Gsdmd^{-/-} mice. n=6-10. (FIG. 2B) Induction of IL-18 secretion by Alu RNA in WT and Gsdmd^{-/-} mouse RPE cells. n=3; *P<0.05. Error bars denote SD. (FIG. 2C) Alu RNA activates caspase-1 (p10 subunit) in WT but not in Gsdmd^{-/-} mouse RPE cells. (FIG. 2D) Similar activation of caspase-11 by Alu RNA in WT and Gsdmd^{-/-} mouse RPE cells. (FIG. 2E) Gasdermin D p30 cleavage occurs in WT mouse BMDMs following intracellular LPS exposure but not in primary human RPE cells and WT mouse RPE cells following Alu RNA exposure. (FIG. 2F) In vivo subretinal transfection of plasmids expressing wild type gasdermin D (pGSDMD-WT) or mutant gasdermin D incapable of undergoing p30 cleavage (pGSDMD-D276A), but not of control vector (pNull), restored Alu RNA-induced RPE degeneration in Gsdmd^{-/-} mice. n=4-5. (FIG. 2G) Resistance of Gsdmd^{-/-} mice to Alu RNA-induced RPE degeneration was overcome by subretinal administration of recombinant mature IL-18 (recIL-18) or enforced expression by pIL-18ss. n=7-8. (FIG. 2H) GSDMD mRNA abundance was greater in the RPE of human AMD eyes (n=7) than in healthy age-matched control eyes (n=8); *P<0.05. Geometric means with 95% confidence intervals are depicted. (FIG. 2I) Increased immunolocalization of gasdermin D in the RPE of human geographic atrophy eyes compared to age-matched healthy controls. Representative immunoblots of three independent experiments and densitometric analysis (mean (SEM)) are shown. Binary and morphometric quantification of RPE degeneration are shown (*, P<0.05; **, P<0.01; * * *, P<0.001). PM, polymegethism (mean (SEM)).

FIGS. 3A-3L. Non-canonical inflammasome activation and RPE degeneration induced by Alu RNA is mediated via interferon signaling. (FIG. 3A) PAlu induces RPE degeneration in WT but not Ifnar1^{-/-} mice. n=7. (FIG. 3B) Caspase-11 activation by Alu RNA is abrogated in Ifnar1^{-/-} mouse RPE cells. (FIG. 3C) IFN-β treatment of human RPE cells induces caspase-4 abundance. (FIG. 3D) PAlu induces IFN-β secretion by human RPE cells. n=3; *P<0.05. Error bars denote SD. (FIG. 3E) Induction of STAT2 phosphorylation in human RPE cells by DICER1 antisense oligonucleotides (DICER1 AS) and pAlu. (FIG. 3F) pAlu induces RPE degeneration in WT but not Irf3^{-/-} or Stat2^{-/-} mice. n=6-7. (FIG. 3G) IFN-β neutralizing antibody, but not isotype control IgG, confers protection against Alu RNA-induced RPE degeneration in WT mice. n=10. (FIG. 3H) Increased immunolocalization of IFN-β in the RPE of human geographic atrophy eyes compared to age-matched unaffected controls. (FIG. 3I) Increased abundance of IFN-β mRNA in the RPE of human GA eyes compared to age-matched healthy controls, n=4, *P<0.05. Error bars denote SEM. Representative immunoblots of three independent experiments and densitometric analysis (mean (SEM)) are shown. Binary and morphometric quantification of RPE degeneration are shown (*, P<0.05; **, P<0.01; * * *, P<0.001). PM, polymegethism (mean (SEM)).

FIGS. 4A-4I. CGAS driven signaling licenses non-canonical inflammasome and RPE degeneration. (FIG. 4A) Alu RNA-induced Ifnb mRNA in WT mouse RPE cells is markedly depressed in Mb21d1^{-/-} mouse RPE cells. n=3, *P<0.05. Error bars denote SEM. (FIG. 4B) Caspase-1 activation (p20 subunit) by pAlu in WT but not Mb21d1^{-/-} mouse RPE cells. (FIG. 4C) Caspase-11 activation (p30 subunit) by pAlu in WT but not Mb21d1^{-/-} mouse RPE cells. (FIG. 4D) Impaired IL-18 secretion in Alu RNA-treated Mb21d1^{-/-} compared to WT mouse RPE cells. n=3; *P<0.05, Error bars denote SD. (FIG. 4E) DICER1 antisense oligonucleotides (DICER1 AS)-induced IFNB mRNA in human RPE cells is markedly depressed by cGAS shRNA (shcGAS). n=3; *P<0.05. Error bars denote SEM. (FIG. 4F) Alu RNA-induced phosphorylation of STAT2 and activation of caspase-4 (p30 subunit) and caspase-1 (p20 subunit) are suppressed in human RPE cells treated with cGAS shRNA. (FIG. 4G) Alu RNA induces RPE degeneration in WT but not Mb21d1^{-/-} mice. n=6-8. (FIG. 4H) Reconstitution of Mb21d1^{-/-} mice with in vivo transfection of Flag-cGAS, but not of control Flag-GFP, restored their susceptibility to Alu RNA-induced RPE degeneration, n=7. (FIG. 4I) Resistance of Mb21d1^{-/-} mice to Alu RNA-induced RPE degeneration was overcome by subretinal administration of recombinant IFN-β (rec IFN-β) or enforced expression by pIFNB. n=10-12. Representative immunoblots of three independent experiments and densitometric analysis (mean (SEM)) are shown. Binary and morphometric quantification of RPE degeneration are shown (*, P<0.05; * *, P<0.01; * * *, P<0.001). PM, polymegethism (mean (SEM)).

FIGS. 5A-5E. CGAS in geographic atrophy and cGAS signaling in RPE degeneration. (FIG. 5A) Increased immunolocalization of cGAS in the RPE of human geographic atrophy eyes compared to age-matched healthy controls. (FIG. 5B) Caspase-1 activation (p20 subunit) by pAlu is impaired in Tmem173^{-/-} mouse RPE cells. (FIG. 5C) Caspase-11 activation (p30 subunit) by Alu RNA is impaired in Tmem173^{-/-} mouse RPE cells. (FIG. 5D) Alu RNA induces RPE degeneration in WT but not Tmem173^{-/-} mice. n=6-10. (FIG. 5E) Resistance of Tmem173^{-/-} mice to Alu RNA-induced RPE degeneration was overcome by subretinal administration of recombinant IFN-β (rec IFN-β) or enforced expression by pIFNB. n=8. Representative immunoblots of three independent experiments and densitometric analysis (mean (SEM)) are shown. Binary and morphometric quantification of RPE degeneration are shown (*, P<0.05; **, P<0.01; * * *, P<0.001). PM, polymegethism (mean (SEM)).

FIGS. 6A-6J. MtDNA in non-canonical inflammasome activation and RPE degeneration. (FIG. 6A) Increased cytosolic mitochondrial DNA (mtDNA) abundance in Alu RNA-treated human RPE cells. n=3; *P<0.05, Error bars denote SEM. (FIG. 6B) Alu RNA induces engagement of cGAS with mtDNA as demonstrated in a ChIP-like cGAS pull-down experiment. n=3; *P<0.05, Error bars denote SEM. (FIG. 6C) Alu RNA induces cytosolic release of mtDNA in WT but not Ppif^{-/-} mouse RPE cells. (FIG. 6D) Alu RNA induces RPE degeneration in WT but not Ppif^{-/-} mice. n=6-12. (FIG. 6E) Alu RNA induces caspase-1 activation in WT but not Ppif^{-/-} mouse RPE cells. (FIG. 6F) Alu RNA induces caspase-11 activation in WT but not Ppif^{-/-} mouse RPE cells. (FIG. 6G) Caspase-4 activation by Alu RNA is abrogated in Rho⁰ human ARPE19 cells lacking mtDNA. (FIG. 6H) Alu RNA-induced secretion of IL-18 is blunted in Rho⁰ human ARPE 19 cells. n=3; *P<0.05, Error bars denote SD. (FIG. 6I) Alu RNA-induced IFN-β secretion is blunted in Rho⁰ human ARPE19 cells. n=3; *P<0.05, Error bars denote SD. (FIG. 6J) Resistance of Ppif^{-/-} mice to Alu RNA-induced RPE degeneration was overcome by subretinal administration of recombinant IFN-β (rec IFN-β) or enforced expression by pIFNB. n=10-11. All immunoblots are representative of three independent experiments. Representative immunoblots of three independent experiments and densitometric analysis (mean (SEM)) are shown. Binary and morphometric quantification of RPE degeneration are shown (*, P<0.05; **, P<0.01; * * *, P<0.001). PM, polymegethism (mean (SEM)).

FIGS. 7A-7H (also referred to as Supplementary FIG. 1). Caspase-4/11 is required for Alu-induced RPE degeneration. (FIG. 7A) Alu RNA induces caspase-4 mRNA in human RPE cells. n=3; *P<0.05, Error bars denote SEM. (FIG. 7B) Protein lysates from RPE of human donor eyes were immunoblotted with an isotype antibody (control for anti-caspase-4 immunoblotting antibody in FIG. 1a). No immunoreactive bands were observed in isotype control immunoblot. (FIG. 7C) Human RPE cells mock treated or stimulated with Alu RNA. Protein lysates were immunoblotted with secondary antibody alone, an isotype antibody, or an anti-caspase-4 antibody; caspase-4 activation (p30 subunits) was observed in Alu RNA stimulated cells; no bands were observed in secondary alone or isotype control immunoblots. Specific bands of interest are indicated by arrowheads. (FIG. 7D) Caspase-4 activation (p30 subunit) in human RPE cells treated with DICER1 antisense oligonucleotides (DICER1 AS) compared to control oligonucleotides (Ctrl AS) is abrogated by simultaneous treatment with Alu RNA antisense oligonucleotides (Alu AS) but not by scrambled oligonucleotide (Scr). (FIG. 7E) Activation of caspase-11 (p26 subunit) in mouse RPE cells by Alu RNA and pAlu. (FIG. 7F) pAlu, but not the control plasmid (pNull), induces RPE degeneration in WT but not Casp11^{-/-} mice, n=6-10. (FIG. 7G) 129S6 mice which carry a caspase-11 inactivating passenger mutation are not susceptible to Alu RNA- or pAlu-induced RPE degeneration, n=5-7. (FIG. 7H) Dicer1 siRNA induces RPE degeneration in WT but not 129S6 mice, n=7-10. Binary and morphometric quantification of RPE degeneration are shown (*, P<0.05; * *, P<0.01; * * *, P<0.001). PM, polymegethism (mean (SEM)).

FIGS. 8A-8E (also referred to as Supplementary FIG. 2). Caspase-11 is required for Alu RNA-induced caspase-1 activation and RPE degeneration. (FIG. 8A) Casp11^{-/-} mouse RPE cells reconstituted via transfecting caspase-11 expression plasmid or control plasmid were stimulated withAlu RNA. Caspase-1 activity was assessed using CASPALUX®1-E₁D₂. Alu RNA-induced relative caspase-1 activity is presented; error bars denote SEM. (FIG. 8B) Caspase-1 activation (p10 subunit) by Alu RNA in Casp11^{-/-} mouse RPE cells is increased following lentiviral vector delivered caspase-11 reconstitution. (FIG. 8C) pAlu induces RPE degeneration in WT mice but not in caspase-1 and caspase-11 deficient mice (Casp1^{-/-} Casp11^{129mt/129mt}) or in Casp1^{-/-} Casp11^{129mt/129mt} mice expressing functional mouse caspase-11 from a bacterial artificial chromosome transgene (Casp1^{-/-} Casp11^{129mt/129mt} Casp11^{Tg}), n=5-6. (FIG. 8D) Alu RNA-induced caspase-11 activation is impaired in P2rx7^{-/-} mouse RPE cells compared to WT mouse RPE cells. (FIG. 8E) Alu RNA-induced caspase-11 activation is not impaired in Pycard^{-/-} mouse RPE cells. In fundus photographs, RPE degeneration is outlined by blue arrowheads. Representative immunoblots of three independent experiments and densitometric analysis (mean (SEM)) are shown. Binary and morphometric quantification of RPE degeneration are shown (*, P<0.05; **, P<0.01; ***, P<0.001). PM, polymegethism (mean (SEM)).

FIGS. 9A-9F (also referred to as Supplementary FIG. 3). Cellular morphometry analysis. Wild-type mouse RPE flat mount images were analyzed in semi-automated fashion by 3 masked raters. (FIG. 9A) Box plot of RPE cell size (μm²) in mouse RPE flat mounts after mock injection or Alu RNA injection into the vitreous humor. (FIG. 9B) Box plot of RPE cell size (μm²) in mouse RPE flat mounts after pNull (null plasmid) injection or pAlu (plasmid encoding Alu) injection into the subretinal space. (FIG. 9C) Box plot of RPE cell density (cells/mm²) in mouse RPE flat mounts after pNull (null plasmid) injection or pAlu (plasmid encoding Alu) injection into the subretinal space. (FIG. 9D) Box plot of RPE cell density (cells/mm²) in mouse RPE flat mounts after mock injection or Alu RNA injection into the vitreous humor. (FIG. 9E) Box plot of polymegethism, a measure of variability of cell size (%) in mouse RPE flat mounts after pNull (null plasmid) injection or pAlu (plasmid encoding Alu) injection into the subretinal space. (FIG. 9F) Box plot of polymegethism, a measure of variability of cell size (%) in mouse RPE flat mounts after mock injection or Alu RNA injection into the vitreous humor. There was a significant difference in cell density (FIGS. 9A and 9B), mean cell area (FIGS. 9C and 9D), and polymegethism (FIGS. 9E and 9F) between Alu RNA- and pAlu-treated eyes compared with their respective controls. ***, P<0.0001, t test. Box plot shows median (red line), interquartile range (box), and the extremes (line segments).

FIGS. 10A-10D (also referred to as Supplementary FIG. 4). Increased abundance of phospholipid oxidation products in Alu RNA-stimulated RPE cells. (FIG. 10A) Brief schematic highlighting select products of PAPC (1-palmitoyl-2-arachidonyl-sn-glycero-3-phosphocholine) oxidation, collectively referred to as oxPAPC. (FIG. 10B) Representative mass scan of pure unoxidized PAPC and oxPAPC using an ABI Sciex 4000 QTrap mass spectrometer. (FIG. 10C) Representative mass scan of oxPAPC, formed from air-oxidized PAPC. (FIG. 10D) Quantification of individual species of oxPAPC by liquid chromatography-mass spectrometry. Human RPE cells stimulated with Alu RNA had higher levels of PGPC (1-palmitoyl-2-glutaroyl-sn-glycero-3-phosphocholine) and LysoPC (1-palmitoyl-2-hydroxy-sn-glycero-3-phosphocholine) levels, indicative of extended oxidation, concomitant with a trending decrease of precursor PAPC and intermediate POVPC. n=6; * P<0.05, Error bars denote standard error.

FIGS. 11A-11D (also referred to as Supplementary FIG. 5). Gasdermin D is required for Alu-induced RPE degeneration. (FIG. 11A) pAlu induces RPE degeneration in WT but not Gsdmd^{-/-} mice, n=7-9. (FIG. 11B) Dicer1 siRNA induces RPE degeneration in WT but not Gsdmd^{-/-} mice, n=7-10. (FIG. 11C) Alu RNA induced secretion of IL-18 in Gsdmd^{-/-} mouse RPE cells reconstituted with either pGSDMD-WT or pGSDMD-D276A. (FIG. 11D) IL-8, IL-6, and MIP-la mRNA abundance in the RPE was not different between human AMD eyes (n=4) and healthy age-matched control eyes (n=4); Error bars denote SEM. Binary and morphometric quantification of RPE degeneration are shown (*, P<0.05; **, P<0.01; * * *, P<0.001). PM, polymegethism (mean (SEM)).

FIGS. 12A and 12B (also referred to as Supplementary FIG. 6), each comprising 16 panels. Alu RNA induces apoptotic cell death in human RPE cells. Human RPE cells mock treated or stimulated with Alu RNA were incubated with FITC-conjugated annexin V (green) and propidium iodide (PI, red). Staining by annexin V and PI uptake was monitored by time-lapse imaging. Representative images at various time points showing annexin V and PI staining is presented for (FIG. 12A) Alu RNA stimulated and (FIG. 12B) mock treated human RPE cells.

FIGS. 13A and 13B (also referred to as Supplementary FIG. 7). Alu RNA induces apoptotic RPE cell death in mice and in human cell culture. (FIG. 13A) Annexin V (periwinkle blue) and propidium iodide (PI; red) staining of RPE flat mounts from WT mice treated with Alu RNA. The area of Alu RNA-induced RPE degeneration contained predominantly annexin-V⁺ PI⁻ cells, consistent with apoptosis. The RPE in regions of the eye distant from the site of Alu RNA exposure was healthy and negative for both annexin V and propidium iodide staining. (FIG. 13B) Immunoblots show that cleaved caspase-3 and PARP-1 were increased in human RPE cells exposed to Alu RNA. ON, optic nerve.

FIG. 14 (also referred to as Supplementary FIG. 8), comprises 18 panels. Resistance of the RPE in Gsdmd^{-/-} mice to Alu RNA-induced apoptotic cell death is overcome by IL-18. Lack of annexin V (periwinkle blue; middle column) and propidium iodide (PI; red, left column) staining in RPE flat mounts of Gsdmd^{-/-} mice treated with Alu RNA. Administration of recombinant mature IL-18 led to the appearance of numerous annexin-V⁺ PI⁻ cells in the area of RPE degeneration. ON, optic nerve.

FIGS. 15A-15E (also referred to as Supplementary FIG. 9). Interferon signaling in RPE toxicity. (FIG. 15A) Induction of IRF3 and STAT2 phosphorylation by Alu RNA in human RPE cells. (FIG. 15B) pAlu induces STAT2 phosphorylation in WT but not Ifnar1^{-/-} mouse RPE cells. (FIG. 15C) Dicer1 siRNA, but not control siRNA, induces RPE degeneration in WT but not Stat2^{-/-} mice. n=7. (FIG. 15D) Alu RNA induces RPE degeneration in WT but not Stat2^{-/-} mice. n=6-7. (FIG. 15E) Alu RNA induces caspase-11 activation in WT but not Stat2^{-/-} mouse RPE cells. Expression of representative immunoblots of three independent experiments and densitometric analysis (mean (SEM)) are shown. Binary and morphometric quantification of RPE degeneration are shown (*, P<0.05; **, P<0.01; ***, P<0.001). PM, polymegethism (mean (SEM)).

FIGS. 16A-16I (also referred to as Supplementary FIG. 10). cGAS driven signaling licenses non-canonical inflammasome. (FIG. 16A) Alu RNA increased abundance of cGAS mRNA in human RPE cells. n=3; * P<0.05, Error bars denote SEM. (FIG. 16B) Increased cGAS protein abundance in human RPE cells exposed to Alu expression plasmid (pAlu) and Alu RNA. (FIG. 16C) Alu RNA-induced caspase-1 activation (p20 subunit) is suppressed in Mb21d1^{-/-} mouse RPE cells compared to WT cells. (FIG. 16D) Secretion of IL-18 by canonical inflammasome activating monosodium urate (MSU) crystals is unaffected in Casp11^{-/-} and Mb21d1^{-/-} mouse RPE cells. (FIG. 16E) Confirmation of shRNA-mediated knockdown of cGAS mRNA in human RPE cells transfected with control or DICER1 targeted antisense oligonucleotides. Representative data of three experiments presented. Error bars denote SEM of technical replicates. (FIG. 16F) Confirmation of antisense oligonucleotide-mediated DICER1 knockdown in human RPE cells transduced with lentiviral vectors expressing control and cGAS targeted shRNA sequences. Representative data of three experiments presented. Error bars denote SEM of technical replicates. (FIG. 16G) pAlu induces RPE degeneration in WT but not Mb21d1^{-/-} mice. n=6-8. (FIG. 16H) Dicer1 siRNA induces RPE degeneration in WT but not Mb21d1^{-/-} mice. n=6-9. (FIG. 16I) Alu RNA-induced Ifnb mRNA expression is increased in Mb21d1^{-/-} mouse RPE cells reconstituted with cGAS expression plasmid compared to empty vector treated cells. n=3. Error bars denote SEM. Representative immunoblots of three independent experiments and densitometric analysis (mean (SEM)) are shown. Binary and morphometric quantification of RPE degeneration are shown (*, P<0.05; **, P<0.01; * * *, P<0.001). PM, polymegethism (mean (SEM)).

FIGS. 17A-17D (also referred to as Supplementary FIG. 11). CGAS expression validation, and STING involvement in Alu RNA-induced IRF3 activation. (FIG. 17A) Immunoblot shows successful enforced cGAS expression in the RPE, in in vitro and in vivo reconstitution experiments using plasmid transfection described in Supplementary FIG. 101 and FIG. 4h, respectively. (FIGS. 17B and 17D) Increased immunofluorescent localization of phosphorylated IRF3 (pIRF3) in the nucleus of wild-type (FIG. 17B) compared to Tmem173^{-/-} (FIG. 17D) mouse RPE cells. (FIG. 17C) Immunoblot shows Alu RNA-induced pIRF3 in wild-type mouse RPE cells is impaired in Tmem173^{-/-} mouse RPE cells.

FIGS. 18A-18H (also referred to as Supplementary FIG. 12). Activation of cGAS driven signaling by Alu RNA is mediated by cytosolic mtDNA. (FIG. 18A) Increased cytosolic mtDNA abundance in human RPE cells treated with DICER1 antisense oligonucleotides (DICER1 AS) compared to control oligonucleotides (Ctr AS). n=3; * P<0.05, error bars denote SEM. (FIG. 18B) Western blot shows the purity of the mitochondria-free cytosolic fractions used for measuring mtDNA abundance in cytosolic fractions; VDAC-1 is a mitochondrial marker. (FIG. 18C) Stimulation of HA-cGAS cells (Mb21d1 MEF reconstituted with HA-mouse cGAS) with poly I:C (which does not activate cGAS signaling) does not induce engagement of cGAS with mtDNA, as demonstrated in a ChIP-like cGAS pull-down experiment. (FIG. 18D) Positive control for ChIP-like cGAS pull-down assay wherein HA-cGAS cells were transfected with plasmid DNA pUC19, followed by assaying of interaction between cGAS and pUC19 plasmid. (FIG. 18E) Subretinal administration of mtDNA induces RPE degeneration in WT but not Mb21d1^{-/-} mice. n=4-6. (FIG. 18F) Alu RNA-induced abundance of Ifnb mRNA is reduced in Mb21d1^{-/-} mouse RPE cells. (FIG. 18G) Mitochondrial membrane potential (ΔΨm), assessed by the potential-sensitive fluorochrome JC-1, was significantly reduced by Alu RNA in WT but not Ppif^{-/-} mouse RPE cells. Cyclosporin A (CsA) inhibited the reduction in ΔΨm in WT cells. n=5, *P<0.05, error bars denote SEM. (FIG. 18H) Mitochondrial permeabilization, assessed by the quenching of calcein-AM fluorescence by cobalt chloride, was significantly increased by Alu RNA in WT but not Ppif^{-/-} mouse RPE cells. Cyclosporin A (CsA) blocked the mitochondrial permeabilization in WT cells. n=5, *P<0.05, error bars denote SEM. Binary and morphometric quantification of RPE degeneration are shown (*, P<0.05; **, P<0.01; ***, P<0.001). PM, polymegethism (mean (SEM)).

FIGS. 19A-19C (also referred to as Supplementary FIG. 13). Macrophages and microglia are dispensable for Alu RNA-induced RPE degeneration. (FIG. 19A) Alu RNA induces RPE degeneration in WT mice treated with clodronate liposomes. n=6. (FIG. 19B) Alu RNA induces RPE degeneration in Cx3cr1^{CreER} ROSA-DTA mice treated with tamoxifen. n=4-11. (FIG. 19C) Tamoxifen-induced depletion of microglia in Cx3cr1^{CreER} ROSA-DTA mice was confirmed by staining for microglial marker F4/80 superimposed with endothelial cell staining with isolectin B4 in retinal flat mounts. Binary and morphometric quantification of RPE degeneration are shown (*, P<0.05; * *, P<0.01; * * *, P<0.001). PM, polymegethism (mean (SEM)).

FIG. 20 (also referred to as Supplementary FIG. 14). Activation of caspase-1 by Alu RNA in bone marrow derived macrophages (BMDMs) is dependent on caspase-11, cGAS, and gasdermin D. Immunoblots show Alu RNA induced caspase-1 activation (Casp1 p10) in WT but not Casp11^{-/-}, Mb21d1^{-/-}, or Gsdmd^{-/-} BMDMs.

FIG. 21 (also referred to as Supplementary FIG. 15). Schematic Model of the presently disclosed cGAS-mediated licensing of non-canonical NLRP3 inflammasome activation by DICER deficit/Alu RNA. Elevated Alu RNA triggers release of mitochondrial DNA (mtDNA) into the cytosol. Cytosolic mtDNA subsequently activates cGAS-driven type I interferons (IFNs). The resulting IFN signaling via interferon-α/β receptor (IFNAR) and STATs triggers caspase-4/11 priming and activation that, in turn, dictates gasdermin D and NLRP3 inflammasome-mediated secretion of IL-18. Secreted IL-18 drives RPE degeneration via a mechanism involving Myd88, FAS/FasL, and caspase-8^{2,21}. Three different RPE cells are depicted in the schematic model to illustrate the mechanism of Alu RNA-induced inflammasome activation and the autocrine and paracrine IL-18 signaling leading to RPE cell death via Myd88, Fas/FasL, Caspase-8, and Caspase-3.

### DETAILED DESCRIPTION

### Abbreviation and Acronyms

AMD age-related macular degeneration
AS antisense
BMDM bone marrow derived macrophage
caspase a family of cysteine-aspartic acid protease proteases
caspase-4 a human caspase (the murine homolog is caspase 11)
caspase-11 a murine caspase (the human homolog is caspase 4)
cGAS cyclic GMP-AMP synthase
CFH complement factor H
CsA Cyclosporin A (CAS No. 59865-13-3)
DAMPs damage-associated molecular patterns
DICER1 AS DICER1 antisense
GSDMD Gasdermin D
HTRA1 high-temperature requirement factor A-1
IFN interferon
IFN-β interferon beta (also referred to as IFNB)
IFNAR interferon-α/β receptor
IRF3 interferon regulatory factor 3
LPS lipopolysaccharide
LRR leucine-rich repeat
LysoPC 1-palmitoyl-2-hydroxy-3-phosphatidylcholine
MRM multiple reaction monitoring
MSU monosodium urate
mtDNA mitochondrial DNA
mPTP mitochondrial permeability transition pore (a complex of proteins often referred to as just “a” protein)
NLR nucleotide-binding domain and leucine-rich repeat containing protein
NLRP3 NLR family pyrin domain containing 3; also known as NALP3 and cryoporin;
ON optic nerve
pALU plasmid-mediated enforced expression of Alu RNA
PAMP pathogen-associated molecular pattern
PAPC 1-palmitoyl-2-arachidonoyl-3-phosphatidylcholine
PARP-1 poly(ADP-ribose) polymerase 1
PGPC 1-palmitoyl-2-glutaryl-3-phosphatidylcholine
POVPC 1-palmitoyl-2-(5-oxovaleryl)-3-phosphatidylcholine
PI propidium iodide
PM polymegethism
PPIF peptidyl-prolyl cis-trans isomerase F
RPE retinal pigmented epithelium
Scr scrambled oligonucleotide
shcGAS cGAS shRNA
shRNA short hairpin RNA
siRNA small interfering RNA
STING stimulator of interferon genes
TAM tamoxifen
WT wild type
ZO-1 zonula occludens-1

### Definitions

In describing and claiming the invention, the following terminology will be used in accordance with the definitions set forth below.

The articles “a” and “an” are used herein to refer to one or to more than one (i.e. to at least one) of the grammatical object of the article. By way of example, “an element” means one element or more than one element.

The term “about”, as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about”.

The terms “additional therapeutically active compound” or “additional therapeutic agent”, as used in the context of the present invention, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As used herein, the terms “administration of” and or “administering” a compound should be understood to mean providing a compound of the invention or a prodrug of a compound of the invention to a subject in need of treatment.

As used herein, an “agonist” is a composition of matter which, when administered to a mammal such as a human, enhances or extends a biological activity attributable to the level or presence of a target compound or molecule of interest in the mammal.

The term “alterations in peptide structure” as used herein refers to changes including, but not limited to, changes in sequence, and post-translational modification.

The term “the alternative, non-canonical inflammasome signaling molecules, protein complex, or signal transduction pathways in retinal pigment epithelium (RPE)” as used herein refers to the pathway and molecules disclosed herein and to their signal transduction pathways. Regarding the signal transduction pathways of the alternative, non-canonical inflammasome, this includes both upstream and downstream regulation that can be regulated or inhibited in RPE to inhibit or prevent RPE degeneration and age-related macular degeneration or its progression to geographic atrophy.

An “antagonist” is a composition of matter which when administered to a mammal such as a human, inhibits a biological activity attributable to the level or presence of a compound or molecule of interest in the mammal.

As used herein, “alleviating a disease or disorder symptom”, means reducing the severity of the symptom or the frequency with which such a symptom is experienced by a patient, or both.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The term “amino acid” is used interchangeably with “amino acid residue”, and may refer to a free amino acid and to an amino acid residue of a peptide. It will be apparent from the context in which the term is used whether it refers to a free amino acid or a residue of a peptide.

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

Amino acids have the following general structure:

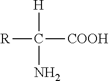

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “antagomir” refers to a small RNA or DNA (or chimeric) molecule to antagonize endogenous small RNA regulators like microRNA (miRNA). These antagonists bear complementary nucleotide sequences for the most part, which means that antagomirs should hybridize to the mature microRNA (miRNA). They prevent other molecules from binding to a desired site on an mRNA molecule and are used to silence endogenous microRNA (miR). Antagomirs are therefore designed to block biological activity of these post-transcriptional molecular switches. Like the preferred target ligands (microRNA, miRNA), antagomirs have to cross membranes to enter a cell. Antagomirs are also known as anti-miRs or blockmirs.

An “antagonist” is a composition of matter which when administered to a mammal such as a human, inhibits a biological activity attributable to the level or presence of a compound or molecule of interest in the mammal.

The term “antibody”, as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies.

An “antibody heavy chain”, as used herein, refers to the larger of the two types of polypeptide chains present in all antibody molecules.

An “antibody light chain”, as used herein, refers to the smaller of the two types of polypeptide chains present in all antibody molecules.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

The term “antigenic determinant” as used herein refers to that portion of an antigen that makes contact with a particular antibody (i.e., an epitope). When a protein or fragment of a protein, or chemical moiety is used to immunize a host animal, numerous regions of the antigen may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “antimicrobial agents” as used herein refers to any naturally-occurring, synthetic, or semi-synthetic compound or composition or mixture thereof, which is safe for human or animal use as practiced in the methods of this invention, and is effective in killing or substantially inhibiting the growth of microbes. “Antimicrobial” as used herein, includes antibacterial, antifungal, and antiviral agents.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

An “aptamer” is a compound that is selected in vitro to bind preferentially to another compound (for example, the identified proteins herein). Often, aptamers are nucleic acids or peptides because random sequences can be readily generated from nucleotides or amino acids (both naturally occurring or synthetically made) in large numbers but of course they need not be limited to these.

As used herein, the term “attach”, or “attachment”, or “attached”, or “attaching”, used herein interchangeably with “bind”, or “binding” or “binds’ or “bound” refers to any physical relationship between molecules that results in forming a stable complex, such as a physical relationship between a ligand, such as a peptide or small molecule, with a “binding partner” or “receptor molecule”. The relationship may be mediated by physicochemical interactions including, but not limited to, a selective noncovalent association, ionic attraction, hydrogen bonding, covalent bonding, Van der Waals forces or hydrophobic attraction.

As used herein, the term “avidity” refers to a total binding strength of a ligand with a receptor molecule, such that the strength of an interaction comprises multiple independent binding interactions between partners, which can be derived from multiple low affinity interactions or a small number of high affinity interactions.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the term “biologically active fragments” or “bioactive fragment” of the proteins or polypeptides encompasses natural or synthetic portions of the full-length protein that are capable of specific binding to their natural ligand or of performing the function of the protein.

The term “biological sample”, as used herein, refers to samples obtained from a subject, including, but not limited to, skin, hair, tissue, blood, plasma, cells, sweat and urine.

As used herein, the term “biopsy tissue” refers to a sample of tissue that is removed from a subject for the purpose of determining if the sample contains cancerous tissue. In some embodiment, biopsy tissue is obtained because a subject is suspected of having cancer. The biopsy tissue is then examined for the presence or absence of cancer.

As used herein, the term “carrier molecule” refers to any molecule that is chemically conjugated to a molecule of interest.

Caspase-4 is a human protein and is known as caspase-11 in the mouse. Caspases (cysteine-aspartic proteases/cysteine aspartases/cysteine-dependent aspartate-directed proteases) are a family of protease enzymes playing a role in programmed cell death. There is a precursor and isoforms for caspase-4. Isoform alpha is known as the canonical sequence.

Caspase-11 is a mouse protein and is known as caspase-4 in humans.

The terms “cell”, “cell line”, and “cell culture” as used herein may be used interchangeably. All of these terms also include their progeny, which are any and all subsequent generations. It is understood that all progeny may not be identical due to deliberate or inadvertent mutations.

As used herein, the term “chemically conjugated”, or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homolog thereof that competes with another peptide for its cognate binding site.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound”, as used herein, refers to any type of substance or agent that is commonly considered a drug, or a candidate for use as a drug, as well as combinations and mixtures of the above, and can also include biologics that are used for a treatment or effect in the context of the uses described herein.

As used herein, the term “conservative amino acid substitution” is defined herein as an amino acid exchange within one of the following five groups:
I. Small aliphatic, nonpolar or slightly polar residues: Ala, Ser, Thr, Pro, Gly
II. Polar, negatively charged residues and their amides: Asp, Asn, Glu, Gln
III. Polar, positively charged residues: His, Arg, Lys
IV. Large, aliphatic, nonpolar residues: Met, Leu, Ile, Val, Cys
V. Large, aromatic residues: Phe, Tyr, Trp

A “control” cell is a cell having the same cell type as a test cell. The control cell may, for example, be examined at precisely or nearly the same time the test cell is examined. The control cell may also, for example, be examined at a time distant from the time at which the test cell is examined, and the results of the examination of the control cell may be recorded so that the recorded results may be compared with results obtained by examination of a test cell.

A “test” cell is a cell being examined.

“Cytokine”, as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets and effector activities of these cytokines have been described.

As used herein, a “derivative” of a compound refers to a chemical compound that may be produced from another compound of similar structure in one or more steps, as in replacement of H by an alkyl, acyl, or amino group.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

As used herein, the term “effector domain” refers to a domain capable of directly interacting with an effector molecule, chemical, or structure in the cytoplasm which is capable of regulating a biochemical pathway.

The term “elixir”, as used herein, refers in general to a clear, sweetened, alcohol-containing, usually hydroalcoholic liquid containing flavoring substances and sometimes active medicinal agents.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “enhancer” is a DNA regulatory element that can increase the efficiency of transcription, regardless of the distance or orientation of the enhancer relative to the start site of transcription.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′-ATTGCC-5′ and 3′-TATGGC-5′ share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator using the BLAST tool at the NCBI website. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

As used herein, the term “inhaler” refers both to devices for nasal and pulmonary administration of a drug, e.g., in solution, powder and the like. For example, the term “inhaler” is intended to encompass a propellant driven inhaler, such as is used to administer antihistamine for acute asthma attacks, and plastic spray bottles, such as are used to administer decongestants.

The term “inhibit”, as used herein, refers to the ability of a compound, agent, or method to reduce or impede a described function, level, activity, rate, etc., based on the context in which the term “inhibit” is used. The term also refers to inhibiting any metabolic or regulatory pathway which can regulate the synthesis, levels, activity, or function of a protein, mRNA, or other molecule of interest. Preferably, inhibition is by at least 10%. The term “inhibit” is used interchangeably with “reduce” and “block”.

The term “inhibit a complex”, as used herein, refers to inhibiting the formation of a complex or interaction of two or more proteins, as well as inhibiting the function or activity of the complex. The term also encompasses disrupting a formed complex. However, the term does not imply that each and every one of these functions must be inhibited at the same time.

The term “inhibit a protein”, as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting or applying” includes administration of a compound of the invention by any number of routes and means including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, or rectal means.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

A “ligand” is a compound that specifically binds to a target receptor or target molecule.

A “receptor” or target molecule is a compound that specifically binds to a ligand.

A ligand or a receptor “specifically binds to” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically binds under hybridization conditions to a compound polynucleotide comprising a complementary sequence; an antibody specifically binds under immunoassay conditions to an antigen bearing an epitope against which the antibody was raised.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions, e.g., a nucleic acid molecule that hybridizes to one complementary sequence at the 5′ end and to another complementary sequence at the 3′ end, thus joining two non-complementary sequences.

“Malexpression” of a gene means expression of a gene in a cell of a patient afflicted with a disease or disorder, wherein the level of expression (including non-expression), the portion of the gene expressed, or the timing of the expression of the gene with regard to the cell cycle, differs from expression of the same gene in a cell of a patient not afflicted with the disease or disorder. It is understood that malexpression may cause or contribute to the disease or disorder, be a symptom of the disease or disorder, or both.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present.

The term “method of identifying peptides in a sample”, as used herein, refers to identifying small and large peptides, including proteins.

Micro-RNAs are generally about 16-25 nucleotides in length. In one aspect, miRNAs are RNA molecules of 22 nucleotides or less in length. These molecules have been found to be highly involved in the pathology of several types of cancer. Although the miRNA molecules are generally found to be stable when associated with blood serum and its components after EDTA treatment, introduction of locked nucleic acids (LNAs) to the miRNAs via PCR further increases stability of the miRNAs. LNAs are a class of nucleic acid analogues in which the ribose ring is “locked” by a methylene bridge connecting the 2′-O atom and the 4′-C atom of the ribose ring, which increases the molecule's affinity for other molecules. miRNAs are species of small non-coding single-stranded regulatory RNAs that interact with the 3′-untranslated region (3′-UTR) of target mRNA molecules through partial sequence homology. They participate in regulatory networks as controlling elements that direct comprehensive gene expression. Bioinformatics analysis has predicted that a single miRNA can regulate hundreds of target genes, contributing to the combinational and subtle regulation of numerous genetic pathways.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid”, “DNA”, “RNA” and similar terms also include nucleic acid analogs, i.e. analogs having other than a phosphodiester backbone. For example, the so-called “peptide nucleic acids”, which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject. These can be used as controls, as can standard samples comprising known amounts of the target to be detected or measured.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides.

As used herein, the term “peptide ligand” (or the word “ligand” in reference to a peptide) refers to a peptide or fragment of a protein that specifically binds to a molecule, such as a protein, carbohydrate, and the like. A receptor or binding partner of the peptide ligand can be essentially any type of molecule such as polypeptide, nucleic acid, carbohydrate, lipid, or any organic derived compound. Specific examples of ligands are peptide ligands of the present inventions.

The term “per application” as used herein refers to administration of a drug or compound to a subject.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application.

As used herein, “pharmaceutical compositions” include formulations for human and veterinary use.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A “prophylactic” treatment is a treatment administered to a subject who does not exhibit signs of a disease or exhibits only early signs of the disease for the purpose of decreasing the risk of developing pathology associated with the disease.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

As used herein, the term “providing a prognosis” refers to providing information regarding the impact of the presence of or susceptibility to, for example, age-related macular degeneration (e.g., as determined by the diagnostic methods of the present invention) on a subject's future health (e.g., age, risks such as smoking, the likelihood of getting AMD or geographic atrophy).

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. In particular, purified sperm cell DNA refers to DNA that does not produce significant detectable levels of non-sperm cell DNA upon PCR amplification of the purified sperm cell DNA and subsequent analysis of that amplified DNA. A “significant detectable level” is an amount of contaminate that would be visible in the presented data and would need to be addressed/explained during analysis of the forensic evidence.

“Pyroptosis” as used herein is a highly inflammatory form of programmed cell death that occurs most frequently upon infection with intracellular pathogens and is likely to form part of the antimicrobial response. “Pyroptotic” refers to an agent or process that can induce pyroptosis.

A “recombinant adeno-associated viral (AAV) vector comprising a regulatory element active in RPE cells” refers to an AAV that has been constructed to comprise a new regulatory element to drive expression or tissue-specific expression in RPE of a gene of choice or interest. As described herein such a constructed vector may also contain at least one promoter and optionally at least one enhancer as part of the regulatory element, and the recombinant vector may further comprise additional nucleic acid sequences, including those for other genes, including therapeutic genes of interest.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

A “recombinant cell” is a cell that comprises a transgene. Such a cell may be a eukaryotic or a prokaryotic cell. Also, the transgenic cell encompasses, but is not limited to, an embryonic stem cell comprising the transgene, a cell obtained from a chimeric mammal derived from a transgenic embryonic stem cell where the cell comprises the transgene, a cell obtained from a transgenic mammal, or fetal or placental tissue thereof, and a prokaryotic cell comprising the transgene.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl-β-galactoside to the medium (Gerhardt et al., eds., 1994, Methods for General and Molecular Bacteriology, American Society for Microbiology, Washington, DC, p. 574).

A “sample”, as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

As used herein, the term “secondary antibody” refers to an antibody that binds to the constant region of another antibody (the primary antibody).

A “short hairpin RNA” (shRNA) as used herein is an artificial RNA molecule with a tight hairpin turn that can be used to silence target gene expression via RNA interference (RNAi). Expression of shRNA in cells is typically accomplished by delivery of plasmids or through viral or bacterial vectors. shRNA is an advantageous mediator of RNAi in that it has a relatively low rate of degradation and turnover.

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

By “small interfering RNAs (siRNAs)” is meant, inter alia, an isolated dsRNA molecule comprised of both a sense and an anti-sense strand. In one aspect, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

As used herein, the term “solid support” relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

By the term “specifically binds to”, as used herein, is meant when a compound or ligand functions in a binding reaction or assay conditions which is determinative of the presence of the compound in a sample of heterogeneous compounds, or it means that one molecule, such as a binding moiety, e.g., an oligonucleotide or antibody, binds preferentially to another molecule, such as a target molecule, e.g., a nucleic acid or a protein, in the presence of other molecules in a sample.

The terms “specific binding” or “specifically binding” when used in reference to the interaction of a peptide (ligand) and a receptor (molecule) also refers to an interaction that is dependent upon the presence of a particular structure (i.e., an amino sequence of a ligand or a ligand binding domain within a protein); in other words the peptide comprises a structure allowing recognition and binding to a specific protein structure within a binding partner rather than to molecules in general. For example, if a ligand is specific for binding pocket “A”, in a reaction containing labeled peptide ligand “A” (such as an isolated phage displayed peptide or isolated synthetic peptide) and unlabeled “A” in the presence of a protein comprising a binding pocket A the unlabeled peptide ligand will reduce the amount of labeled peptide ligand bound to the binding partner, in other words a competitive binding assay.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, preferably a human.

As used herein, a “subject in need thereof” is a patient, animal, mammal, or human, who will benefit from the method of this invention.

As used herein, the term “subject at risk for AMD” refers to a subject with one or more risk factors for developing AMD. Risk factors may include, but are not limited to, gender, age, genetic predisposition, environmental expose, and lifestyle.

As used herein, a “substantially homologous amino acid sequences” includes those amino acid sequences which have at least about 95% homology, preferably at least about 96% homology, more preferably at least about 97% homology, even more preferably at least about 98% homology, and most preferably at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. Preferably, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; preferably in 7% (SDS), 0.5 M NaPO₄, 1 mM EDTA at 50° C. with washing in 1×SSC, 0.1% SDS at 50° C.; preferably 7% SDS, 0.5 M NaPO₄, 1 mM EDTA at 50° C. with washing in 0.5×SSC, 0.1% SDS at 50° C.; and more preferably in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the present invention.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “susceptible to age-related macular degeneration or geographic atrophy” or “age-related macular degeneration or geographic atrophy” is not meant to infer that geographic atrophy is not a form or stage of age-related macular degeneration, but that a treatment or diagnosis can be in reference to the two.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

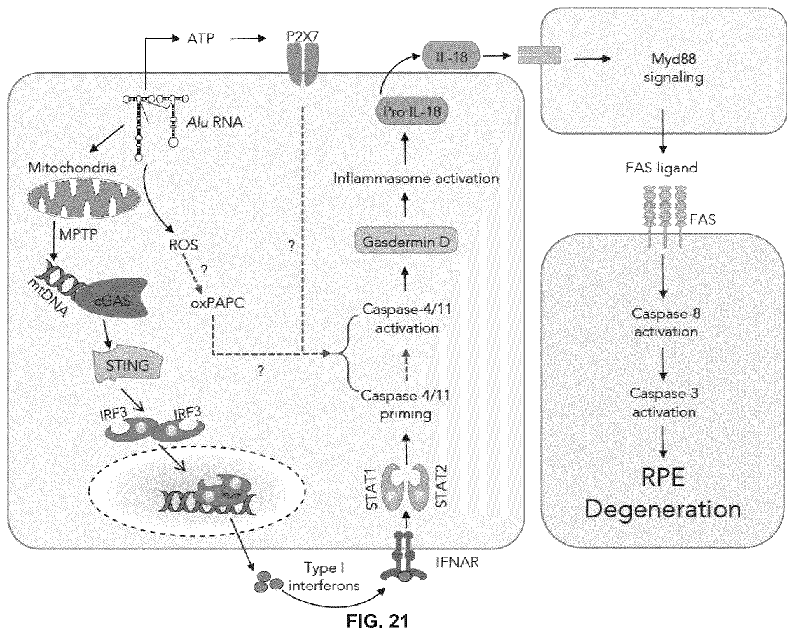

By “targeting at least one of the alternative, non-canonical inflammasome signaling molecules or pathways in retinal pigment epithelium (RPE)” is meant either targeting one of the molecules directly (see Schematic of FIG. 21), such as with an inhibitor, or by targeting part of the molecule's signal transduction pathway that also impacts the alternative, non-canonical inflammasome signaling in RPE that leads to degeneration. The targeting and treatment encompass administering an effective amount of an inhibitor of noncanonical-inflammasome activation in RPE. Also, reference to a “protein complex” includes the mPTP, which is also sometimes just referred to as a protein. The targeting can include the use of agents that are stimulatory or inhibitory, depending on the context of the particular molecule being targeted and the result desired.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The term to “treat”, as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

The term “transfection” is used interchangeably with the terms “gene transfer” “, transformation”, and “transduction”, and means the intracellular introduction of a polynucleotide. “Transfection efficiency” refers to the relative amount of the transgene taken up by the cells subjected to transfection. In practice, transfection efficiency is estimated by the amount of the reporter gene product expressed following the transfection procedure.

The term “transgene” is used interchangeably with “inserted gene”, or “expressed gene” and, where appropriate, “gene”. “Transgene” refers to a polynucleotide that, when introduced into a cell, is capable of being transcribed under appropriate conditions so as to confer a beneficial property to the cell such as, for example, expression of a therapeutically useful protein. It is an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “transgenic mammal” means a mammal, the germ cells of which comprise an exogenous nucleic acid.

As used herein, a “transgenic cell” is any cell that comprises a nucleic acid sequence that has been introduced into the cell in a manner that allows expression of a gene encoded by the introduced nucleic acid sequence.

Where appropriate, the term “transgene” should be understood to include a combination of a coding sequence and optional non-coding regulatory sequences, such as a polyadenylation signal, a promoter, an enhancer, a repressor, etc.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system.

Expression vectors include all those known in the art, such as cosmids, plasmids (e.g., naked or contained in liposomes) and viruses that incorporate the recombinant polynucleotide.

### Embodiments

The present application discloses a new and unexpected pathway that regulates RPE cell degeneration and provides compositions and method for regulating that pathway. That is, it is disclosed herein that RPE degeneration is regulated by the non-canonical inflammasome, aspects of which were previously known only to function in inflammation subsequent to infections.

Useful inhibitory molecules of the invention for inhibiting the activity and levels of the target molecules described herein include, but are not limited to, drugs, antibodies and biologically active fragments and homologs thereof, monoclonal antibodies and biologically active fragments and homologs thereof, humanized antibodies, antisense oligonucleotides, shRNA, siRNA, aptamers, and anti-oxidants. By biologically active is meant that they have the intended function as described herein for inhibiting the target molecule of interest.

The present invention also provides for homologs of proteins and peptides. Homologs can differ from naturally occurring proteins or peptides by conservative amino acid sequence differences or by modifications which do not affect sequence, or by both.

For example, conservative amino acid changes may be made, which although they alter the primary sequence of the protein or peptide, do not normally alter its function. To that end, 10 or more conservative amino acid changes typically have no effect on protein function.

In another embodiment, the methods of the present invention may be used to prevent or treat macular degeneration. In one embodiment, macular degeneration is characterized by damage to or breakdown of the macula, which in one embodiment, is a small area at the back of the eye. In one embodiment, macular degeneration causes a progressive loss of central sight, but not complete blindness. In one embodiment, macular degeneration is of the dry type, while in another embodiment, it is of the wet type. In one embodiment, the dry type is characterized by the thinning and loss of function of the macula tissue. In one embodiment, the wet type is characterized by the growth of abnormal blood vessels behind the macula. In one embodiment, the abnormal blood vessels hemorrhage or leak, resulting in the formation of scar tissue if untreated. In some embodiments, the dry type of macular degeneration can turn into the wet type. In one embodiment, macular degeneration is age-related, which in one embodiment is caused by an ingrowth of choroidal capillaries through defects in Bruch's membrane with proliferation of fibrovascular tissue beneath the retinal pigment epithelium.

Diagnosis of AMD using the compositions and methods of the present invention can be coupled with known methods. For example, the early and intermediate stages of AMD usually start without symptoms. A comprehensive dilated eye exam can detect AMD. The eye exam may include the following:
1. Visual acuity test. An eye chart measure is used to measure vision at distances.
2. Dilated eye exam. The eye care professional places drops in the eyes to widen or dilate the pupils. This provides a better view of the back of the eye. Using a special magnifying lens, he or she then looks at your retina and optic nerve for signs of AMD and other eye problems.
3. Amsler grid. The eye care professional also may ask you to look at an Amsler grid. Changes in central vision may cause the lines in the grid to disappear or appear wavy, a sign of AMD.
4. Fluorescein angiogram. In this test, which is performed by an ophthalmologist, a fluorescent dye is injected into the subject's arm. Pictures are taken as the dye passes through the blood vessels in the eye. This makes it possible to see leaking blood vessels, which occur in a severe, rapidly progressive type of AMD (see below).
5. Optical coherence tomography. This technique uses light waves, and can achieve very high-resolution images of any tissues that can be penetrated by light-such as the eyes.

There are also multiple methods available for predicting susceptibility to age-related macular degeneration or geographic atrophy. As mentioned above, for example, “age-related macular degeneration or geographic atrophy” is not meant to infer that geographic atrophy is not a form or stage of age-related macular degeneration, but that a treatment or diagnosis can be in reference to the two. Methods and biomarkers are available for predicting whether a subject is susceptible to AMD, including, for example, the existence genetic variants of complement factor H (CFH) and high-temperature requirement factor A-1 (HTRA1) that can be detected, smoking, and, of course, age. When a subject has been tested and is diagnosed or predicted to be susceptible to an RPE disease or disorder, one or more of the therapeutic agents of the invention can be administered prophylactically.

Caspases (cysteine-aspartic proteases/cysteine aspartases/cysteine-dependent aspartate-directed proteases) are a family of protease enzymes playing a role in programmed cell death. There is a precursor and isoforms for caspase-4. Isoform alpha is known as the canonical sequence.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides or antibody fragments which have been modified using ordinary molecular biological techniques so as to improve their resistance to proteolytic degradation or to optimize solubility properties or to render them more suitable as a therapeutic agent. Homologs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Substantially pure protein or peptide obtained as described herein may be purified by following known procedures for protein purification, wherein an immunological, enzymatic, or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

RNA interference (RNAi) is a commonly used method to regulate gene expression. This effect is often achieved by using small interfering RNA (siRNA) or short hairpin RNA (shRNA). Applying these small RNAs to cells under in vitro conditions is relatively easy but this application under in vivo conditions is difficult due to various issues, such as short life of these molecules and their inability to access target cells. In one aspect, these issues can be solved by plasmid or vector-mediated delivery.

In one aspect, AAV can be used. The natural tissue tropism of the various AAV serotypes can be exploited to favor gene delivery to one organ over another. This tropism is based on the viral capsids recognizing specific viral receptors expressed on specific cell types, thus allowing a degree of cell specific targeting within a given organ. Cell-specific expression may be further aided by the use of tissue-specific promoters conferring gene expression restricted to a specific cell type. This is desirable for gene therapy applications targeting organ specific diseases, as this will help avoid any possible harmful side effects due to gene expression in off target organs.

In one embodiment, an isolated nucleic acid of the invention is encoded by a vector.

In one aspect, the isolated nucleic acid is operably-linked to a cell-specific promoter.

In one aspect, a lipid vehicle comprises said isolated nucleic acid.

In one aspect, the vectors further comprise a gene of interest, which may be a therapeutic gene. The regulatory element may include an enhancer and/or a promoter. The combination of specific vectors, including AAV vectors, enhancers, promoters, and therapeutic genes, and fragments and homologs thereof that are used can be modified to ensure a high rate of targeting cells and tissues of interest and expression of therapeutic genes and genes of interest in the target cell of tissue of interest.

The present invention does not just encompass administering pharmaceutical compositions comprising an effective amount of a compound of the invention. The present invention further encompasses targeting RPE cells.

In one embodiment, the present invention provides for the administration of at least one miRNA, including pre-miRNA and mature miRNA, or a mimic thereof. “miRNA mimics” are chemically synthesized nucleic acid based molecules, preferably double-stranded RNAs which mimic mature endogenous miRNAs after transfection into cells. In one aspect, an antagonist of the miRNA can be used. In another aspect, an agonist of the miRNA can be used. The type of regulator can be chosen depending on the role of the molecule(s) or pathway to be targeted in the RPE cell.

miRNAs are transcribed by RNA polymerase II (pol II) or RNA polymerase III and arise from initial transcripts, termed primary miRNA transcripts (pri-miRNAs), that are generally several thousand bases long. Pri-miRNAs are processed in the nucleus by the RNase Drosha into about 70- to about 100-nucleotide hairpin-shaped precursors (pre-miRNAs). Following transport to the cytoplasm, the hairpin pre-miRNA is further processed by Dicer to produce a double-stranded miRNA. The mature miRNA strand is then incorporated into the RNA-induced silencing complex (RISC), where it associates with its target mRNAs by base-pair complementarity. In the relatively rare cases in which a miRNA base pairs perfectly with an mRNA target, it promotes mRNA degradation. More commonly, miRNAs form imperfect heteroduplexes with target mRNAs, affecting either mRNA stability or inhibiting mRNA translation.

In one aspect, an miR-specific inhibitor may be an anti-miRNA (anti-miR) oligonucleotide (for example, see WO2005054494).

An administered miRNA may be the naturally occurring miRNA or it may be an analogue or homologue of the miRNA. In one aspect, the miRNA, or analogue or homologues, are modified to increase the stability thereof in the cellular milieu. In an alternative aspect the miRNA is encoded by an expression vector and may be delivered to the target cell in a liposome or microvesicle.

Amino Acid Substitutions

In certain embodiments, the disclosed methods and compositions may involve preparing peptides with one or more substituted amino acid residues. In various embodiments, the structural, physical and/or therapeutic characteristics of peptide sequences may be optimized by replacing one or more amino acid residues.

In one embodiment, the invention encompasses the substitution of a serine or an alanine residue for a cysteine residue in a peptide of the invention. Support for this includes what is known in the art. For example, see the following citation for justification of such a serine or alanine substitution: Kittlesen et al., 1998 Human melanoma patients recognize an HLA-A1-restricted CTL epitope from tyrosinase containing two cysteine residues: implications for tumor vaccine development J Immunol., 60, 2099-2106.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

The skilled artisan will be aware that, in general, amino acid substitutions in a peptide typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art. For example, one can make the following isosteric and/or conservative amino acid changes in the parent polypeptide sequence with the expectation that the resulting polypeptides would have a similar or improved profile of the properties described above:

Substitution of alkyl-substituted hydrophobic amino acids: including alanine, leucine, isoleucine, valine, norleucine, S-2-aminobutyric acid, S-cyclohexylalanine or other simple alpha-amino acids substituted by an aliphatic side chain from C1-10 carbons including branched, cyclic and straight chain alkyl, alkenyl or alkynyl substitutions.

Substitution of aromatic-substituted hydrophobic amino acids: including phenylalanine, tryptophan, tyrosine, biphenylalanine, 1-naphthylalanine, 2-naphthylalanine, 2-benzothienylalanine, 3-benzothienylalanine, histidine, amino, alkylamino, dialkylamino, aza, halogenated (fluoro, chloro, bromo, or iodo) or alkoxy-substituted forms of the previous listed aromatic amino acids, illustrative examples of which are: 2-,3- or 4-aminophenylalanine, 2-,3- or 4-chlorophenylalanine, 2-,3- or 4-methylphenylalanine, 2-,3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2,3, or 4-biphenylalanine, 2′-, 3′-, or 4′-methyl-2, 3 or 4-biphenylalanine, and 2- or 3-pyridylalanine.

Substitution of amino acids containing basic functions: including arginine, lysine, histidine, ornithine, 2,3-diaminopropionic acid, homoarginine, alkyl, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma′-diethyl-homoarginine. Included also are compounds such as alpha methyl arginine, alpha methyl 2,3-diaminopropionic acid, alpha methyl histidine, alpha methyl ornithine where alkyl group occupies the pro-R position of the alpha carbon. Also included are the amides formed from alkyl, aromatic, heteroaromatic (where the heteroaromatic group has one or more nitrogens, oxygens, or sulfur atoms singly or in combination) carboxylic acids or any of the many well-known activated derivatives such as acid chlorides, active esters, active azolides and related derivatives) and lysine, ornithine, or 2,3-diaminopropionic acid.

Substitution of acidic amino acids: including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids.

Substitution of side chain amide residues: including asparagine, glutamine, and alkyl or aromatic substituted derivatives of asparagine or glutamine.

Substitution of hydroxyl containing amino acids: including serine, threonine, homoserine, 2,3-diaminopropionic acid, and alkyl or aromatic substituted derivatives of serine or threonine. It is also understood that the amino acids within each of the categories listed above can be substituted for another of the same group.

For example, the hydropathic index of amino acids may be considered (Kyte & Doolittle, 1982, J. Mol. Biol., 157:105-132). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within +/−2 is preferred, within +/−1 are more preferred, and within +/−0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see, e.g., Chou & Fasman, 1974, Biochemistry, 13:222-245; 1978, Ann. Rev. Biochem., 47: 251-276; 1979, Biophys. J., 26:367-384).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) leu, ile, val; Arg (R) gln, asn, lys; Asn (N) his, asp, lys, arg, gln; Asp (D) asn, glu; Cys (C) ala, ser; Gln (Q) glu, asn; Glu (E) gln, asp; Gly (G) ala; His (H) asn, gln, lys, arg; Ile (I) val, met, ala, phe, leu; Leu (L) val, met, ala, phe, ile; Lys (K) gln, asn, arg; Met (M) phe, ile, leu; Phe (F) leu, val, ile, ala, tyr; Pro (P) ala; Ser (S), thr; Thr (T) ser; Trp (W) phe, tyr; Tyr (Y) trp, phe, thr, ser; Val (V) ile, leu, met, phe, ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. (See, e.g., PROWL Rockefeller University website). For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. (Id.) Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix and Risler matrix (Idem.)

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

The invention is also directed to methods of administering the compounds of the invention to a subject.

Pharmaceutical compositions comprising the present compounds are administered to an individual in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means.

The present invention is also directed to pharmaceutical compositions comprising the peptides of the present invention. More particularly, such compounds can be formulated as pharmaceutical compositions using standard pharmaceutically acceptable carriers, fillers, solublizing agents and stabilizers known to those skilled in the art.

The invention also encompasses the use pharmaceutical compositions of an appropriate compound, homolog, fragment, analog, or derivative thereof to practice the methods of the invention, the composition comprising at least one appropriate compound, homolog, fragment, analog, or derivative thereof and a pharmaceutically-acceptable carrier.

The pharmaceutical compositions useful for practicing the invention may be administered to deliver a dose of between 1 ng/kg/day and 100 mg/kg/day. Pharmaceutical compositions that are useful in the methods of the invention may be administered systemically in oral solid formulations, ophthalmic, suppository, aerosol, topical or other similar formulations. In addition to the appropriate compound, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer an appropriate compound according to the methods of the invention.

The invention encompasses the preparation and use of pharmaceutical compositions comprising a compound useful for treatment of the conditions, disorders, and diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology. A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose.

Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively).

Known emulsifying agents include, but are not limited to, lecithin and acacia. Known preservatives include, but are not limited to, methyl, ethyl, or n-propyl para hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil in water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may also be prepared, packaged, or sold in a formulation suitable for rectal administration, vaginal administration, parenteral administration

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butanediol, for example.

Other acceptable diluents and solvents include, but are not limited to, Ringer's solution, isotonic sodium chloride solution, and fixed oils such as synthetic mono or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, PA, which is incorporated herein by reference.

Typically, dosages of the compound of the invention which may be administered to an animal, preferably a human, range in amount from 1 μg to about 100 g per kilogram of body weight of the subject. While the precise dosage administered will vary depending upon any number of factors, including but not limited to, the type of animal and type of disease state being treated, the age of the animal and the route of administration. In one embodiment, the dosage of the compound will vary from about 10 μg to about 10 g per kilogram of body weight of the animal. In another embodiment, the dosage will vary from about 10 mg to about 1 g per kilogram of body weight of the subject.

The compound may be administered to a subject as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the subject, etc.

In one aspect, additional therapeutic agents of the pharmaceutical compositions of the invention are anti-ischemia agents. One of ordinary skill in the art will appreciate that the composition may further comprise an effective amount of at least one additional therapeutic agents which may be useful for the type of injury, disease, or disorder being treated. Additional therapeutic agents include, but are not limited to, anesthetic, analgesic, antimicrobial, steroid, growth factor, cytokine, and anti-inflammatory agents. Useful anesthetic agents include benzocaine, lidocaine, bupivocaine, dibucaine, mepivocaine, etidocaine, tetracaine, butanilicaine, and trimecaine.

In another aspect, the agent is at least one analgesic. In yet another aspect, the agent is an additional therapeutic drug.

In a further aspect, the additional therapeutic agent is an antimicrobial agent. In one aspect, the antimicrobial agent is an antibacterial agent. In another aspect, the antimicrobial agent is an antifungal agent. In yet another aspect, the antimicrobial agent is an antiviral agent. Antimicrobial agents useful in the practice of the invention include, but are not limited to, silver sulfadiazine, Nystatin, Nystatin/triamcinolone, Bacitracin, nitrofurazone, nitrofurantoin, a polymyxin (e.g., Colistin, Surfactin, Polymyxin E, and Polymyxin B), doxycycline, antimicrobial peptides (e.g., natural and synthetic origin), Neosporin (i.e., Bacitracin, Polymyxin B, and Neomycin), Polysporin (i.e., Bacitracin and Polymyxin B). Additional antimicrobials include topical antimicrobials (i.e., antiseptics), examples of which include silver salts, iodine, benzalkonium chloride, alcohol, hydrogen peroxide, and chlorhexidine. It may be desirable for the antimicrobial to be other than Nystatin.

In another aspect, the agent is selected from aspirin, pentoxifylline, and clopidogrel bisulfate, or other angiogenic, or a rheologic active agent.

The invention also includes a kit comprising a compound of the invention and an instructional material which describes administering the composition to a cell or a tissue of a subject. In another embodiment, this kit comprises a (preferably sterile) solvent suitable for dissolving or suspending the composition of the invention prior to administering the compound to the subject. The invention also provides an applicator, and an instructional material for the use thereof.

### Examples

### Methods-

Mice

All animal experiments were approved by institutional review committees and performed in accordance with the Association for Research in Vision and Ophthalmology Statement for the Use of Animals in Ophthalmic and Visual Research. Both male and female mice between 6-10 weeks of age were used in the study. Wild-type C57BL/6J, Ppif^{-/-}, P2rx7^{-/-}, Stat2^{-/-} mice were purchased from The Jackson Laboratory. Gsdmd^{-/-}, Pycard^{-/-}, Casp11^{-/-}, Casp1^{-/-} Casp11^{129mt/129mt} and Casp1^{-/-} Casp11^{129mt/129mt} Casp11^{Tg} mice, described elsewhere¹⁻⁴, were a generous gift from V. M Dixit (Genentech). Caspase-11 deficient mouse transgenically expressing human caspase-4 (Casp11^{-/-} hCasp4^{Tg}) were described earlier⁵. Wild type 129S6 mice (that carry an inactivating passenger mutation in caspase-11) were purchased from Taconic Biosciences. Ifnar1^{-/-} mice described earlier⁶ were generous gift from M. Aguet. Irf3^{-/-} mice were a generous gift from T. Taniguchi via M. David⁷. Mb21d1^{-/-} mice were generated by K. A. Fitzgerald (University of Massachusetts Medical School) on a C57BL/6 background using cryopreserved embryos obtained from the European Conditional Mouse Mutagenesis Program (EUCOMM)⁸. Tmem173^{-/-} mice were described earlier⁹. For all procedures, anesthesia was achieved by intraperitoneal injection of 100 mg/kg ketamine hydrochloride (Ft. Dodge Animal Health) and 10 mg/kg xylazine (Phoenix Scientific), and pupils were dilated with topical 1% tropicamide and 2.5% phenylephrine (Alcon Laboratories).

Fundus Photography

TRC-50 IX camera (Topcon) linked to a digital imaging system (Sony) was used for fundus photographs of dilated mouse eyes.

Subretinal Injection

Subretinal injections (1 μl) in mice were performed using a Pico-Injector (PLI-100, Harvard Apparatus) or using a 35-gauge needle (Ito Co. Fuji, Japan). In vivo transfection of plasmids expressing Alu sequences (pAlu)^{10,11}, empty control vector (pNull), Flag-cGAS (pFlag-cGAS), Flag-GFP, mouse mature IL-18 (pIL-18ss)^{12,13}, wildtype mouse gasdermin D (pGSDMD-WT), the p30 cleavage incompetent mutant mouse gasdermin D ((pGSMDD-D276A)², IFN-β (Origene Cat #MR226101), or mtDNA (10 ng) was achieved using 10% Neuroporter (Genlantis) as previously described^{14,15}. In vitro transcribed Alu RNA (0.15-0.3 μg/μl), IFN-β neutralizing antibody (10 ng; Abcam Cat #ab24324), control isotype IgG, recombinant IL-18 (100 ng/μl, MBL Cat #B002-5), or IFN-β (500 mUnit/μl, PBL Cat #12410-1) were administered via subretinal injection^{14,15}. Similarly, to knock down Dicer1, 1 μl of cholesterol conjugated siRNA (1 μg/μl) targeting mouse Dicer1 or scrambled control siRNAs were injected. The choice of eye for active versus control injection was chosen randomly.

Assessment of RPE Degeneration

Alu-mediated RPE degeneration was induced by exposing mice to Alu RNA as previously described¹⁴⁻¹⁸. Seven days later, RPE health was assessed by fundus photography and immunofluorescence staining of zonula occludens-1 (ZO-1) on RPE flat mounts (whole mount of posterior eye cup containing RPE and choroid layers). Mouse RPE and choroid flat mounts were fixed with 4% paraformaldehyde or 100% methanol, stained with rabbit polyclonal antibodies against mouse ZO-1 (1:100, Invitrogen), and visualized with Alexa-594 (Invitrogen). All images were obtained by microscopy (model SP-5, Leica; or Axio Observer Z1, Zeiss). Imaging was performed by an operator masked to the group assignments.

Quantification of RPE Degeneration

Binary Assignment: Healthy RPE cells form a polygonal tessellation with a principally hexagonal “honeycomb” formation. RPE degeneration was assessed as a disruption of this uniformity of this polygonal sheet. Thus, RPE health was assessed as the presence or absence of morphological disruption in RPE flat mounts by two independent raters who were masked to the group assignments. Both raters deemed 100% of images as gradable. Inter-rater reliability was measured by agreement on assignments, Pearson coefficient of determination, and Fleiss κ. Fisher's exact test was used to determine statistical significance between the fractions of healthy RPE sheets across groups.

Cellular Morphometry: Quantifying cellular morphometry for hexagonally packed cells was performed in semi-automated fashion by three masked graders by adapting our previous analysis of corneal endothelial cell density¹⁹. As RPE cells when viewed en face typically exhibit a principally hexagonal morphology similar to the corneal endothelium, they readily lend themselves to a similar analysis strategy. We obtained measures of cell size, polymegethism (coefficient of variation of cell size), and cell density. For this analysis, microscopy images of the RPE were captured and transmitted in deidentified fashion to the Doheny Image Reading & Research Lab (DIRRL). Images in which no cell borders could be seen were excluded from further analysis (1.8%). All images were rescaled to 304×446 pixels to permit importation into the Konan CellCheck software (Ver. 4.0.1), a commercial U.S. FDA-cleared software that has been used for registration clinical trials. RPE cell metrics were generated by three certified reading center graders in an independent, masked fashion. Inter-grader agreement was assessed for all three metrics by computing the multiple adjusted coefficient of determination. The previously published center method was utilized which entails the user selecting the center of each identifiable cell in the image²⁰⁻²³. Once the cell centers were defined, the software automatically generated the mean cell area, cell density, and polymegethism values. By default, the Konan software assumes a scaling factor of 124 pixels per 100 μm. Based on the dimensions of the original RPE image (1,024×1,024 pixels, 0.21 μm/pixel), the Konan provided values were converted to the actual physical values in μm.

Human Tissue

All studies on human tissue followed the guidelines of the Declaration of Helsinki. Institutional review boards granted approval for allocation and histological analysis of specimens. Donor eyes from patients with geographic atrophy, an advanced form of AMD or age-matched patients without AMD were obtained from various eye banks. These diagnoses were confirmed by dilated ophthalmic examination prior to acquisition of the tissues or eyes or upon examination of the eye globes post mortem. Enucleated donor eyes isolated within six hours post mortem were immediately preserved in RNALater (ThermoFisher). The neural retina was removed and tissue consisting of macular RPE and choroid were snap frozen in liquid nitrogen. For eyes with GA, the RPE and choroidal tissue was collected comprising of both atrophic and marginal areas.

Immunohistochemistry

Human eyes fixed in 2-4% paraformaldehyde were prepared for immunohistochemistry, and stained as described earlier¹⁴⁻¹⁵. Briefly, immunohistochemical staining of fixed human eyes was performed with rabbit antibody against cGAS (0.1 μg/ml, Sigma-Aldrich, Cat #HPA031700) or interferon β (0.2 μg/ml, Santa Cruz Biotechnology, Cat #sc-20107) and mouse antibody against gasdermin D (1.5 μg/ml, Abcam, Cat #ab57785). Rabbit or mouse IgG controls were used to ascertain the specificity of the staining. Biotin-conjugated secondary antibodies, followed by incubation with VECTASTAIN® ABC reagent and development using Vector Blue (Vector Laboratories) were utilized to detect the bound primary antibody. Slides were washed in PBS, and then mounted in Vectamount (Vector Laboratories). All images were obtained using Zeiss Axio Observer Z1 microscope.

Real-time PCR Total RNA purified from cell using Trizol reagent (Invitrogen) following manufacturer's recommendation was DNase treated and reverse transcribed using QUANTITECT® Reverse Transcription kit (QIAGEN). The RT products (cDNA) were amplified by real-time quantitative PCR (Applied Biosystems 7900 HT Fast Real-Time PCR system) with Power SYBR green Master Mix. Relative gene expression was determined by 2^{-ΔΔCt} t method using 18S rRNA or GAPDH as internal control. Primers were used as described in the provisional patent application from which this application depends and as in Kerur et al., 2018, Nature, the publication resulting from the data in the provisional application. The primers were for human IFNB1, CASP4, DICER, cGAS, 18s rRNA, mitochondrial DNA, GAPDH, and GSDMD, MIP1α, IL6, IL8, and mouse Ifnb1, Gapdh, 18s rRNA, and mitochondrial DNA.

Mitochondrial DNA Preparation

Total DNA extracted from ARPE19 cells was used to PCR-amplify mtDNA segments as described earlier²⁴. The purified mtDNA PCR products were subretinally delivered using 10% Neuroporter (Genlantis) as described above.

Western Blotting

Cell and tissue lysates prepared in RIPA buffer where homogenized by sonication. Protein concentration was determined using Pierce BCA Protein Assay Kit (Thermo Fisher Scientific Inc.). Equal quantity of protein (10-50 μg) prepared in Laemmli buffer were resolved by SDS-PAGE on NOVEX® Tris-Glycine Gels (Invitrogen), and transferred onto Immobilon-FL PVDF membranes (Millipore). The transferred membranes were blocked in ODYSSEY® Blocking Buffer (PBS) or 5% nonfat dry skim milk for 1 hour at RT and then incubated with primary antibody at 4° C. overnight. The immunoreactive bands were visualized with help of species specific secondary antibodies conjugated with IRDYE® or HRP. The blot image were either captured on ODYSSEY® imaging systems or on an autoradiography film. Rabbit polyclonal anti-human and mouse caspase-1 antibodies (1:500, Biovision Cat #3019-100; 1:1000, Invitrogen Cat #AHZ0082; 1:200 Santa Cruz Biotechnology Cat #sc-514), rabbit anti-caspase-1 mAb (1:1000, Abcam Cat #ab108362), anti-human caspase-4 (1:200, Santa Cruz Cat #1229), anti-mouse caspase-11 (1:200, Novus Rat mAb 17D9 Cat #NB120-10454, or 1: 1000 Abcam Rabbit mAb Cat #ab180673 1:500), anti-STAT2 (1:500, Cell Signaling, Cat #72604), anti-pSTAT2 (1:250, Millipore Cat #07-224), anti-human cGAS (1:1000, Cell Signaling Cat #15102), anti-VDAC-1 (1:1000, Cell Signaling Cat ##4661), anti-mouse IRF3 (1:500, Novus Biologicals, Cat #NBP1-78769), anti-phospho-IRF3 (1:500, Cell Signaling Cat #4947S, Cat #29047), anti-HA-tag (1:1000; Cell Signaling Cat #2367), anti-α-tubulin mouse mAb (1:50000, Sigma-Aldrich), anti-β-actin mouse mAb (1:50000, Sigma-Aldrich), anti-vinculin (1:2000, Sigma-Aldrich), anti-cleaved caspase-3 (1:500, Cell Signaling Cat #9661), anti-PARP (1:1000, Cell Signaling Cat #9542), anti-human GSDMD gasdermin D (1:500, Abcam Cat #ab57785) and anti-mouse gasdermin D mAb (1 μg/mL; a generous gift from V. M Dixit (Genentech)) Immunoblotting for activated Caspase 1 in the supernatant was performed as described earlier². Briefly, supernatants collected were briefly spun down to remove floating cells. Proteins from cell-free supernatant were precipitated by adding sodium deoxycholate (0.15% final) followed by adding TCA (7.2% final) and incubating on ice for 30 mins to overnight. Samples were spun down at 13000 g for 30 mins and pellets were washed 2 times with ice-cold acetone. Precipitated proteins solubilized in 4×LDS Buffer with 2-mercaptoethanol was used for immunoblotting.

Cell Culture:

Primary mouse and human RPE cells were isolated as previously described^{25,26}. All cells were maintained at 37° C. and 5% CO₂ environment. Mouse RPE were cultured in Dulbecco Modified Eagle Medium (DMEM) supplemented with 20% FBS and standard penicillin/streptomycin antibiotics concentrations and primary human RPE cells were maintained in DMEM supplemented with 10% FBS and antibiotics. The human RPE cell line ARPE19 and those lacking mitochondrial DNA (Rho⁰ ARPE19) were cultured as described earlier²⁷. Rho⁰ ARPE19 cells were maintained at 37° C. in 24 mM Na₂HCO₃, 10% FBS, 50 μg/ml uridine, 1 mM sodium pyruvate in DMEM-F12 (Gibco, Cat #11320-033) containing pen/strep, Fungizone, and gentamicin. ARPE19 cells were maintained in DMEM-F12 containing pen/strep, Fungizone, and gentamicin. Bone marrow derived macrophages (BMDM) were cultured in DMEM with 10% fetal bovine serum and 20% L929 supernatants. Mb21d1 and HA-cGAS reconstituted Mb21d1 mouse embryonic fibroblasts were cultured in DMEM with 10% FBS and antibiotics²⁸.

Synthesis of In Vitro Transcribed Alu RNA

T7 promoter containing Alu expression plasmid was linearized and used for making in vitro transcribed Alu RNA using AmpliScribe T7-Flash Transcription Kit (Epicenter) following manufacturer's instructions. The resulting Alu RNA was DNase treated and purified using MEGAclear (Ambion), and integrity was monitored by gel electrophoresis^{14,15}.

Transfection

Alu expression plasmid (pAlu), empty vector control (pNull) or in vitro transcribed Alu RNA were transfected in human and mouse RPE using Lipofectamine 2000 (Invitrogen) following the manufacturer's instructions.

LPS Transfection in BMDM

Approximately 2×10⁶ BMDM cells were cultured overnight at 37° C. in a 60-mm dish. After 4-6 h of priming with 1 μg/ml Pam3CSK4 (Invivogen, Cat #tlrl-pms), cells were transfected with LPS (5 μg/ml final concentration, Invivogen, Cat #tlrl-3pelps, ultrapure) with FugeneHD (Promega, Cat #E2311) using standard transfection protocol. 16 h post-transfection, cell lysates were collected and analyzed.

Extraction of Mitochondria-Free Cytosolic Fractions:

Human and mouse RPE cells either mock treated or stimulated with Alu RNA. 24 h post Alu RNA transfection or 48 h post scrambled or DICER1 AS oligonucleotide transfection, cells were harvested by trypsinization. 2×10⁶ cells were used for collecting mitochondria free cytosolic fractions using Mitochondrial Isolation kit (Thermo Scientific Cat #89874). Briefly, cells resuspended in 800 μl of Reagent A and placed on ice for 2 min, the suspension was dounce homogenized (10 strokes) to lyse the cells release nuclei or incubated for 5 min on ice, vortexing every minute after adding 10 μL Reagent B. To this suspension 800 μl Mitochondria Isolation Reagent C was added and the resulting suspension was centrifuged at 700 g for 10 min at 4° C. to pellet the nuclei. The supernatant containing cytoplasmic fraction was centrifuged at 700 g for 10 min at 4° C. total of five times to completely remove nuclei and or any unlysed cells. The resulting nuclei-free cytoplasmic fraction was centrifuged at 13,000 g for 15 min at 4° C. to pellet the mitochondria. The resulting supernatant was further centrifuged at 13,000 g for 15 min at 4° C. a total of six times to remove all the mitochondria. The supernatant was next tested for absence of mitochondria by immunoblotting for the mitochondrial marker protein VDAC and cytosolic marker protein tubulin.

Reconstitution Experiment

Mb21d1^{-/-} mouse RPE cells were transfected with 2 μg cGAS expression plasmid²⁹ or empty vector in a 60 mm dish at 70-80% confluency. 24 h post transfection, cells were plated on 6-well dishes. 24 h post plating, cells were transfected with Alu RNA (50 pmol) or mock transfected using Lipofectamine 2000. 18 h post Alu RNA transfection, cells were collected for RNA extraction to examine induction IFN-β mRNA. For Caspase-11 reconstitution. Casp11^{-/-} mouse RPE cells were transduced with control or caspase-11 expressing lentiviral particles. The transduced cells were allowed to rest for three days and the cells were then plated in 60 mm dish at 70-80% confluency. Control or Caspase-11 reconstituted Casp11^{-/-} cells were mock treated or stimulated with Alu RNA as described above and activation of caspase-1 was assessed by western blotting. For caspase-1 activity assay, Casp11^{-/-} mouse RPE cells transfected with caspase-11 expression plasmid (pCasp11) or empty vector (pNull) were exposed to Alu RNA as described above and caspase-1 activity was assessed using CASPALUX®1-E₁D₂ kit (OncoImmunin Cat #CPL1R1E-5). Quantification of the CaspaLux signal was performed by a blinded operator measuring the integrated density of fluorescent micrographs using Image J software (NIH) and normalizing to the number of cells.

Lentiviral Transduction

Lentivirus articles were either produced by the University of Kentucky Viral Production Core facilities or in house. Lentivirus vector plasmids expressing scrambled sequences or shRNA sequences targeting human caspase-4 and cGAS were purchased (MISSION®shRNA, Sigma-Aldrich) to produce lentiviral particles. Human RPE cells at passage 3 were incubated with lentiviral particles at multiplicity of infection (MOI) of 5 overnight in regular growth media containing polybrene (4 μg/ml). On day 2 cells were washed and incubated in regular growth media allowed to rest for 24 h. Lentivirus transduced cells were then cultured under puromycin (5 μg/ml) selection pressure for 5 days. Knockdown of the target proteins was determined by immunoblotting.

ShRNA Sequences

Some of the shRNAs used in the present application are provided below. All were obtained from Sigma-Aldrich.

shcGAS- Catalog no. TRCN0000150010 (see genes/MB21D1)- SEQ ID NO: 15-
CCGGCCTGCTGTAACACTTCTTATTCTCGAGAATAAGAAGTGTTACAGC
AGGTTTTTTG
shCasp4- Catalog no. TRCN0000003511 (see /genes/CASP4)- SEQ ID NO: 16-
CCGGAGACTATGTAAAGAAAGAGCTCTCGAGAGCTCTTTCTTTACATAG
TCTTTTTT
shSTING- Catalog no. TRCN0000164628 (see /genes/TMEM173)- SEQ ID NO: 17-
CCGGCCAACATTCGCTTCCTGGATACTCGAGTATCCAGGAAGCGAATGT
TGGTTTTTTG
shPPIF- Catalog no. TRCN0000232684 (see /genes/PPIF)- SEQ ID NO: 18-
CCGGCTGTGGCCAGTTGAGCTAATCCTCGAGGATTAGCTCAACTGGCCA
CAGTTTTTG
shGSDMD- Catalog no. TRCN0000178784 (see /genes/GSDMD)- SEQ ID NO: 19-
CCGGCAACCTGTCTATCAAGGACATCTCGAGATGTCCTTGATAGACAGG
TTGTTTTTTG
shIFNB- Catalog no. TRCN0000005803 (see /genes/IFNB1)- SEQ ID NO: 20-
CCGGCAGAGTGGAAATCCTAAGGAACTCGAGTTCCTTAGGATTTCCACT
CTGTTTTT
shIFNAR1- Catalog no. TRCN0000059013 (see /genes/IFNAR1)- SEQ ID NO: 21-
CCGGGCCAAGATTCAGGAAATTATTCTCGAGAATAATTTCCTGAATCTTGG
CTTTTTG

Enzyme-Linked Immunosorbent Assay (ELISA)

Secreted human and mouse interferon-β and IL-18 in the media were detected using the ELISA kits (mouse IFN-β, R&D Systems Cat #42400-1; mouse IL-18, R&D Systems Cat #7625; human IFN-β, R&D Systems Cat #41410; human IL-18, R&D systems Cat #DY318-05) according to the manufacturer's instructions. Primary mouse cells cultured as above. WT, Gsdmd^{-/-}, Casp11^{-/-}, and Mb21d1^{-/-} mouse RPE cells were seeded at a density of 250,000 cells/well in a 12-well plate. When confluency reached 60-70%, cells were transfected with 20 pmol of in vitro transcribed Alu RNA or mock using Lipofectamine 2000 reagent (Life Technologies, Carlsbad, CA) following the manufacturer protocol. Media was collected to detect secreted cytokine content at 8 to 24 h post-transfection. For examining the induction of IL-18 secretion by monosodium urate (MSU) crystals (Invivogen Cat #tlrl-msu), mouse RPE cells were primed with LPS (500 ng/ml) for 6 h and exposed to MSU (250 μg/ml) for 16 h, and media were collected to detect secreted cytokine.

cGAS-mtDNA Interaction Immunoprecipitation Assay

Immortalized cGAS^{-/-} mouse embryonic fibroblasts (MEF) reconstituted with HA-tagged mouse cGAS (HA-cGAS) were described earlier²⁸. Interaction between mtDNA and cGAS was monitored using Express Chromatin Immunoprecipitation Kit (Active Motif, CHIP-IT® Express, cat #53008). Briefly mock, Alu RNA, poly I: C or plasmid DNA (pUC19) transfected HA-cGAS reconstituted cGAS^{-/-} MEFs were fixed with 1% formaldehyde per manufacturer's instructions. The cells were then lysed by sonication in the shearing buffer, centrifuged for 10 min at 18,000 g in a 4° C. microfuge. The supernatant containing the cell lysate was collected and cGAS was immunoprecipitated from each sample using anti-HA tag antibody (Abcam, cat #ab9110). DNA in the IP was eluted, reverse crosslinked and purified using Chromatin IP DNA Purification Kit (Active Motif, cat #58002). Purified DNA was analyzed by qPCR using mouse mtDNA specific primer pairs. Fold enrichment of mtDNA in HA-cGAS IP, in cells exposed to Alu RNA was calculated compared to mock transfected cells.

Quantification of PAPC and oxPAPC by LC-MS

Human RPE cells, mock or stimulated with Alu RNA, were washed with cold PBS and trypsinized at 24 h post-stimulation. The cells were washed with cold PBS and 2×10⁶ cells were used for lipid extraction by a modified Bligh-Dyer extraction method³⁰. Briefly, the cell pellet was manually homogenized and then mixed in a glass tube with 700 μL HPLC-grade chloroform and 300 μL HPLC-grade Methanol (Sigma) supplemented with 0.01% butylated hydroxytoluene (BHT from Sigma) and 189 nmol of the internal standard, di-nonanoyl-phosphatidylcholine (DNPC from Avanti). 1 mL of HPLC-grade water was added and the mixture was vigorously vortexed for 60 sec. Next, the mixture was centrifuged (1,000 rpm for 10 min) to separate the fractions and the organic layer (bottom) was removed and placed into a fresh glass tube. 1 mL of chloroform was added to the aqueous fraction and the extraction was performed once more. The organic layer of the second extraction was combined with the first, and then dried down under nitrogen. Upon complete evaporation of the organic solvent, the lipids were suspended in 300 μL of Solvent A (69% water; 31% methanol; 10 mM ammonium acetate) and stored at −80° C. The determination and quantification of oxidized phosphatidylcholine and phosphatidylethanolamine species was performed by liquid chromatography-linked ESI mass spectrometry, using an ABI Sciex 4000 QTrap. Separation of the phospholipids was achieved by loading samples onto a C8 column (Kinetex 5 μm, 150×4.6 mm from Phenomenex). Elution of the phospholipids was achieved using a binary gradient with Solvent A (69% water; 31% methanol; 10 mM ammonium acetate) and Solvent B (50% methanol; 50% isopropanol; 10 mM ammonium acetate) as the mobile phases. Detection for phosphatidylcholine (PC) was conducted using multiple reaction monitoring (MRM) in positive mode by identification of two transition states for each analyte. Quantification of each analyte was performed based on the peak area of the 184 m/z fragment ion for PC.

Determination of Mitochondrial Permeability Transition Pore Opening.

Mitochondrial permeability transition pore (mPTP) opening in WT and Ppif^{-/-} mouse RPE cells was monitored by the calcein-Co²⁺ technique³¹ using the Mitochondrial Permeability Transition Pore Assay Kit (Biovision Inc Cat #K239-100). Mitochondrial membrane potential was evaluated with the JC-1 fluorochrome-based MITO-ID® Membrane Potential Cytotoxicity Kit (Enzo Cat #ENZ-51019-KP002). mPTP opening was inhibited by performing the above assays using cyclosporine A (10 μM)-containing media. The assay was performed in a 96-well microtiter plate according to the manufacturer's instruction.

Live Cell Imaging

2×10⁴ human RPE cells were plated on each well of an 8-well chambered slide (Thermo Scientific Cat #155411). 18-24 h after plating, cells were transfected with 11.5 pmol in vitro transcribed Alu RNA (using Lipofectamine 2000) in a media supplemented with 2.5 mM CaCl₂) and annexin-V 488 (1:200, Invitrogen V13241) and propidium iodide (1:1500, Invitrogen Cat #P3566). Immediately following transfection, annexin V, propidium iodide, and DIC signals were acquired using a Nikon A1R confocal microscope equipped with an automated stage. Images were captured at 3 min intervals for a total duration of 50 h. Cells were maintained in at 37° C. and 5% CO₂ for the duration of the imaging study via a stage top incubator.

RPE Flat Mount Annexin V/PI-Staining

Mouse RPE/choroid flat mounts prepared in DMEM with 10% FBS were washed with binding buffer once and then incubated with Alexa Fluor™ 647 conjugated Annexin V (Invitrogen) for 15 min. The annexin V stained mouse RPE/choroid flat mounts were fixed with 2% paraformaldehyde for 30 min, stained with propidium iodide (PI) containing RNase (Invitrogen) for 30 min and mounted using ProLong™ Gold Antifade Mountant solution (Thermo Fisher Scientific).

Microglia Depletion

Microglia were depleted via administering tamoxifen to CX3CR1^{CreER}; DTA^{flox} mice which express Cre-ER under control of microglia specific CX3CR1 promoter and also contain flox-STOP-flox diphtheria toxin subunit α (DTA) gene cassette in the ROSA26 locus (DTAV^{flox}). Cx3cr1^{CreER}; DTA^{flox} mice were generated by breeding heterozygous Cx3cr1^{CreER} mice with DTA^{flox} mice (both mice generous gifts from Wai T. Wong and Lian Zhao, NIH). To deplete microglia, tamoxifen was administered to Cx3cr1^{CreER}; DTA^{flox} mice as described earlier³². Briefly, adult 2- to 3-month-old TG mice were administered with tamoxifen (TAM) dissolved in corn oil (Sigma-Aldrich; 500 mg/kg dose of a 20 mg/ml solution) via oral gavage (Schedule: days −2, 0, 5, 10, and 15). On day 11, Alu RNA was delivered via subretinal injection. Alu RNA-induced RPE degeneration was assessed as described above. Microglial depletion was confirmed by staining retinal flat mounts for F4/80. Briefly retinal flat mounts were prepared and fixed in 2% paraformaldehyde for 1 h, and stained with RPE conjugated F4/80 (Bio-Rad, Cat #MCA497PET) and fluorescein labeled Griffonia simplicifolia Lectin isolectin B4 (IB4, Vector Laboratories, Cat #FL-1201). All images were obtained using Zeiss Axio Observer Z1 microscope.

Macrophage Depletion

Depletion of macrophages was achieved via administering clodronate liposomes, which eliminates macrophages, in wild-type mice³³. Briefly, animals received 200 μl clodronate liposomes (Liposoma Cat #LIP-01) through the tail vein on days −2 and day 0. Alu RNA or vehicle control were subretinally injected immediately after the day 0 tail vein injection.

Statistical Analyses

Real-time qPCR and ELISA data are expressed as means±standard error of the mean (SEM) were analyzed using Student t test. The binary readouts of RPE degeneration (i.e., presence or absence of RPE degeneration on fundus and ZO-1-stained flat mount images) were analyzed using Fisher's exact test. Cell morphometry data were assessed using Student t test. P values <0.05 were deemed statistically significant. Sample sizes were selected based on power analysis α=5%; 1−β=80%, such that we could detect a minimum of 50% change assuming a sample SD based on Bayesian inference. Outliers were assessed by Grubbs' test. Based on this analysis no outliers were detected and no data were excluded. Fewer than 5% of subretinal injection recipient tissues were excluded based on prescribed exclusion criteria relating to the technical challenges of this delicate procedure.

### METHODS BIBLIOGRAPHY

1. Mariathasan, S., et al. Differential activation of the inflammasome by caspase-1 adaptors ASC and Ipaf. Nature 430, 213-218 (2004).
2. Kayagaki, N., et al. Caspase-11 cleaves gasdermin D for non-canonical inflammasome signalling. Nature 526, 666-671 (2015).
3. Kayagaki, N., et al. Non-canonical inflammasome activation targets caspase-11. Nature 479, 117-121 (2011).
4. Kanneganti, T. D., et al. Bacterial RNA and small antiviral compounds activate caspase-1 through cryopyrin/Nalp3. Nature 440, 233-236 (2006).
5. Kajiwara, Y., et al. A critical role for human caspase-4 in endotoxin sensitivity. J Immunol 193, 335-343 (2014).
6. Muller, U., et al. Functional role of type I and type II interferons in antiviral defense. Science 264, 1918-1921 (1994).
7. Sato, M., et al. Distinct and essential roles of transcription factors IRF-3 and IRF-7 in response to viruses for IFN-alpha/beta gene induction. Immunity 13, 539-548 (2000).
8. Suschak, J. J., Wang, S., Fitzgerald, K. A. & Lu, S. A cGAS-Independent STING/IRF7 Pathway Mediates the Immunogenicity of DNA Vaccines. J Immunol 196, 310-316 (2016).
9. Jin, L., et al. MPYS is required for IFN response factor 3 activation and type I IFN production in the response of cultured phagocytes to bacterial second messengers cyclic-di-AMP and cyclic-di-GMP. J Immunol 187, 2595-2601 (2011).
10. Bennett, E. A., et al. Active Alu retrotransposons in the human genome. Genome Res 18, 1875-1883 (2008).
11. Shaikh, T. H., Roy, A. M., Kim, J., Batzer, M. A. & Deininger, P. L. cDNAs derived from primary and small cytoplasmic Alu (scAlu) transcripts. J Mol Biol 271, 222-234 (1997).
12. Nagai, H., et al. Gene transfer of secreted-type modified interleukin-18 gene to B16F10 melanoma cells suppresses in vivo tumor growth through inhibition of tumor vessel formation. J Invest Dermatol 119, 541-548 (2002).
13. Hirano, Y., et al. IL-18 is not therapeutic for neovascular age-related macular degeneration. Nat Med 20, 1372-1375 (2014).
14. Kaneko, H., et al. DICER1 deficit induces Alu RNA toxicity in age-related macular degeneration. Nature 471, 325-330 (2011).
15. Tarallo, V., et al. DICER1 loss and Alu RNA induce age-related macular degeneration via the NLRP3 inflammasome and MyD88. Cell 149, 847-859 (2012).
16. Fowler, B. J., et al. Nucleoside reverse transcriptase inhibitors possess intrinsic anti-inflammatory activity. Science 346, 1000-1003 (2014).
17. Kerur, N., et al. TLR-independent and P2X7-dependent signaling mediate Alu RNA-induced NLRP3 inflammasome activation in geographic atrophy. Invest Ophthalmol Vis Sci 54, 7395-7401 (2013).
18. Kim, Y., et al. DICER1/Alu RNA dysmetabolism induces Caspase-8-mediated cell death in age-related macular degeneration. Proc Natl Acad Sci USA 111, 16082-16087 (2014).
19. Huang, J., et al. Comparison of Noncontact Specular and Confocal Microscopy for Evaluation of Corneal Endothelium. Eye Contact Lens (2017).
20. Laing, R. A., Sanstrom, M. M., Berrospi, A. R. & Leibowitz, H. M. Changes in the corneal endothelium as a function of age. Exp Eye Res 22, 587-594 (1976).
21. Doughty, M. J., Muller, A. & Zaman, M. L. Assessment of the reliability of human corneal endothelial cell-density estimates using a noncontact specular microscope. Cornea 19, 148-158 (2000).
22. Inaba, M., Matsuda, M., Shiozaki, Y. & Kosaki, H. Regional specular microscopy of endothelial cell loss after intracapsular cataract extraction: a preliminary report. Acta Ophthalmol (Copenh) 63, 232-235 (1985).
23. Laing, R. A., Sandstrom, M. M. & Leibowitz, H. M. In vivo photomicrography of the corneal endothelium. Arch Ophthalmol 93, 143-145 (1975).
24. Bannwarth, S., Procaccio, V. & Paquis-Flucklinger, V. Rapid identification of unknown heteroplasmic mutations across the entire human mitochondrial genome with mismatch-specific Surveyor Nuclease. Nat Protoc 1, 2037-2047 (2006).
25. Yang, Z., et al. Toll-like receptor 3 and geographic atrophy in age-related macular degeneration. N Engl J Med 359, 1456-1463 (2008).
26. Yang, P., Tyrrell, J., Han, I. & Jaffe, G. J. Expression and modulation of RPE cell membrane complement regulatory proteins. Invest Ophthalmol Vis Sci 50, 3473-3481 (2009).
27. Miceli, M. V. & Jazwinski, S. M. Nuclear gene expression changes due to mitochondrial dysfunction in ARPE-19 cells: implications for age-related macular degeneration. Invest Ophthalmol Vis Sci 46, 1765-1773 (2005).
28. West, A. P., et al. Mitochondrial DNA stress primes the antiviral innate immune response. Nature 520, 553-557 (2015).
29. Sun, L., Wu, J., Du, F., Chen, X. & Chen, Z. J. Cyclic GMP-AMP synthase is a cytosolic DNA sensor that activates the type I interferon pathway. Science 339, 786-791 (2013).
30. Kagan, V. E., et al. Oxidized arachidonic and adrenic PEs navigate cells to ferroptosis. Nat Chem Biol 13, 81-90 (2017).
31. Wang, X., et al. Polydatin, a natural polyphenol, protects arterial smooth muscle cells against mitochondrial dysfunction and lysosomal destabilization following hemorrhagic shock. Am J Physiol Regul Integr Comp Physiol 302, R805-814 (2012).
32. Wang, X., et al. Requirement for Microglia for the Maintenance of Synaptic Function and Integrity in the Mature Retina. J Neurosci 36, 2827-2842 (2016).
33. Sakurai, E., Anand, A., Ambati, B. K., van Rooijen, N. & Ambati, J. Macrophage depletion inhibits experimental choroidal neovascularization. Invest Ophthalmol Vis Sci 44, 3578-3585 (2003).

Summary of some of the useful sequences of the invention

SEQ ID NO: 1:
human cGAS shRNA (TRCN0000146282)-5′-3′
CCGGCTTTGATAACTGCGTGACATACTCGAGTATGTCACGCAGTTATCAA
AGTTTTTTG.
SEQ ID NO: 2:
human cGAS siRNA (SASI_Hs01; see for example Sigma catalog
number for EHU015231-20UG and the target sequence provided).
AAGAAGAAACATGGCGGCTATCCTTCTCTCACATCGAAAAGGAAATTT.
SEQ ID NO: 3:
Human cGAS (cyclic GMP-AMP synthase) Protein-“cyclic GMP-
AMP synthase” [Homo sapiens], GenBank: AGB51853.1, 522 a.a.
MQPWHGKAMQRASEAGATAPKASARNARGAPMDPTESPAAPEAALPKAG
KFGPARKSGSRQKKSAPDTQERPPVRATGARAKKAPQRAQDTQPSDATS
APGAEGLEPPAAREPALSRAGSCRQRGARCSTKPRPPPGPWDVPSPGLP
VSAPILVRRDAAPGASKLRAVLEKLKLSRDDISTAAGMVKGVVDIILLL
RLKCDSAFRGVGLLNTGSYYEHVKISAPNEFDVMFKLEVPRIQLEEYSN
TRAYYFVKFKRNPKENPLSQFLEGEILSASKMLSKFRKIIKEEINDIKD
TDVIMKRKRGGSPAVTLLISEKISVDITLALESKSSWPASTQEGLRIQN
WLSAKVRKQLRLKPFYLVPKHAKEGNGFQEETWRLSFSHIEKEILNNHG
KSKTCCENKEEKCCRKDCLKLMKYLLFQLKFRFKDKKHLDKFSSYHVKT
AFFHVCTQNPQDSQWDRKDLGLCFDNCVTYFLQCLRTEKLENYFIPEFN
LFSSNLIDKRSKEFLTKQIEYERNNEFPVFDEF
SEQ ID NO: 4:
Human cGAS (cyclic GMP-AMP synthase) mRNA- NCBI
Reference Sequence: NM_138441.2, 1802 bp
AGCCTGGGGTTCCCCTTCGGGTCGCAGACTCTTGTGTGCCCGCCAGTAGT
GCTTGGTTTCCAACAGCTGCTGCTGGCTCTTCCTCTTGCGGCCTTTTCCT
GAAACGGATTCTTCTTTCGGGGAACAGAAAGCGCCAGCCATGCAGCCTTG
GCACGGAAAGGCCATGCAGAGAGCTTCCGAGGCCGGAGCCACTGCCCCC
AAGGCTTCCGCACGGAATGCCAGGGGCGCCCCGATGGATCCCACCGAGT
CTCCGGCTGCCCCCGAGGCCGCCCTGCCTAAGGCGGGAAAGTTCGGCCC
CGCCAGGAAGTCGGGATCCCGGCAGAAAAAGAGCGCCCCGGACACCCA
GGAGAGGCCGCCCGTCCGCGCAACTGGGGCCCGCGCCAAAAAGGCCCCT
CAGCGCGCCCAGGACACGCAGCCGTCTGACGCCACCAGCGCCCCTGGGG
CAGAGGGGCTGGAGCCTCCTGCGGCTCGGGAGCCGGCTCTTTCCAGGGC
TGGTTCTTGCCGCCAGAGGGGCGCGCGCTGCTCCACGAAGCCAAGACCT
CCGCCCGGGCCCTGGGACGTGCCCAGCCCCGGCCTGCCGGTCTCGGCCC
CCATTCTCGTACGGAGGGATGCGGCGCCTGGGGCCTCGAAGCTCCGGGC
GGTTTTGGAGAAGTTGAAGCTCAGCCGCGATGATATCTCCACGGCGGCG
GGGATGGTGAAAGGGGTTGTGGACCACCTGCTGCTCAGACTGAAGTGCG
ACTCCGCGTTCAGAGGCGTCGGGCTGCTGAACACCGGGAGCTACTATGA
GCACGTGAAGATTTCTGCACCTAATGAATTTGATGTCATGTTTAAACTGG
AAGTCCCCAGAATTCAACTAGAAGAATATTCCAACACTCGTGCATATTA
CTTTGTGAAATTTAAAAGAAATCCGAAAGAAAATCCTCTGAGTCAGTTTT
TAGAAGGTGAAATATTATCAGCTTCTAAGATGCTGTCAAAGTTTAGGAA
AATCATTAAGGAAGAAATTAACGACATTAAAGATACAGATGTCATCATG
AAGAGGAAAAGAGGAGGGAGCCCTGCTGTAACACTTCTTATTAGTGAAA
AAATATCTGTGGATATAACCCTGGCTTTGGAATCAAAAAGTAGCTGGCC
TGCTAGCACCCAAGAAGGCCTGCGCATTCAAAACTGGCTTTCAGCAAAA
GTTAGGAAGCAACTACGACTAAAGCCATTTTACCTTGTACCCAAGCATG
CAAAGGAAGGAAATGGTTTCCAAGAAGAAACATGGCGGCTATCCTTCTC
TCACATCGAAAAGGAAATTTTGAACAATCATGGAAAATCTAAAACGTGC
TGTGAAAACAAAGAAGAGAAATGTTGCAGGAAAGATTGTTTAAAACTAA
TGAAATACCTTTTAGAACAGCTGAAAGAAAGGTTTAAAGACAAAAAACA
TCTGGATAAATTCTCTTCTTATCATGTGAAAACTGCCTTCTTTCACGTATG
TACCCAGAACCCTCAAGACAGTCAGTGGGACCGCAAAGACCTGGGCCTC
TGCTTTGATAACTGCGTGACATACTTTCTTCAGTGCCTCAGGACAGAAAA
ACTTGAGAATTATTTTATTCCTGAATTCAATCTATTCTCTAGCAACTTAAT
TGACAAAAGAAGTAAGGAATTTCTGACAAAGCAAATTGAATATGAAAG
AAACAATGAGTTTCCAGTTTTTGATGAATTTTGAGATTGTATTTTTAGAA
AGATCTAAGAACTAGAGTCACCCTAAATCCTGGAGAATACAAGAAAAAT
TTGAAAAGGGGCCAGACGCTGTGGCTCAC
SEQ ID NO: 5:
Human Caspase-4-Protein-Full length caspase-4- 377 a.a.-
caspase-4 isoform alpha precursor [Homo sapiens], NCBI
Reference Sequence: NP_001216.1 (for a fragment see
caspase-4 isoform X1 [Homo sapiens], NCBI
Reference Sequence: XP_016873886.1, 286 a.a.)
MAEGNHRKKPLKVLESLGKDFLTGVLDNLVEQNVLNWKEEEKKKYYDAK
TEDKVRVMADSMQEKQRMAGQMLLQTFHNIDQISPNKKAHPNMEAGPPES
GESTDALKLCPHEEFLRLCKERAEEIYPIKERNNRTRLALIICNTEFDHL
PPRNGADFDITGMKELLEGLDYSVDVEENLTARDMESALRAFATRPEHKS
SDSTFLVLMSHGILEGICGTVHDEKKPDVLLYDTIFQIFNNRNCLSLKDK
PKVIIVQACRGANRGELWVRDSPASLEVASSQSSENLEEDAVYKTHVEKD
FIAFCSSTPHNVSWRDSTMGSIFITQLITCFQKYSWCCHLEEVFRKVQQS
FETPRAKAQMPTIERLSMTRYFYLFPGN
SEQ ID NO: 6:
Human Caspase-4 mRNA/nuclcotide-1319 bp, Homo sapiens
caspase 4 (CASP4), transcript variant alpha, mRNA, NCBI
Reference Sequence: NM_001225.3
ATACATAGTTTACTTTCATTTTTGACTCTGAGGCTCTTTCCAACGCTGTAA
AAAAGGACAGAGGCTGTTCCCTATGGCAGAAGGCAACCACAGAAAAAA
GCCACTTAAGGTGTTGGAATCCCTGGGCAAAGATTTCCTCACTGGTGTTT
TGGATAACTTGGTGGAACAAAATGTACTGAACTGGAAGGAAGAGGAAA
AAAAGAAATATTACGATGCTAAAACTGAAGACAAAGTTCGGGTCATGGC
AGACTCTATGCAAGAGAAGCAACGTATGGCAGGACAAATGCTTCTTCAA
ACCTTTTTTAACATAGACCAAATATCCCCCAATAAAAAAGCTCATCCGAA
TATGGAGGCTGGACCACCTGAGTCAGGAGAATCTACAGATGCCCTCAAG
CTTTGTCCTCATGAAGAATTCCTGAGACTATGTAAAGAAAGAGCTGAAG
AGATCTATCCAATAAAGGAGAGAAACAACCGCACACGCCTGGCTCTCAT
CATATGCAATACAGAGTTTGACCATCTGCCTCCGAGGAATGGAGCTGAC
TTTGACATCACAGGGATGAAGGAGCTACTTGAGGGTCTGGACTATAGTG
TAGATGTAGAAGAGAATCTGACAGCCAGGGATATGGAGTCAGCGCTGAG
GGCATTTGCTACCAGACCAGAGCACAAGTCCTCTGACAGCACATTCTTG
GTACTCATGTCTCATGGCATCCTGGAGGGAATCTGCGGAACTGTGCATG
ATGAGAAAAAACCAGATGTGCTGCTTTATGACACCATCTTCCAGATATTC
AACAACCGCAACTGCCTCAGTCTGAAGGACAAACCCAAGGTCATCATTG
TCCAGGCCTGCAGAGGTGCAAACCGTGGGGAACTGTGGGTCAGAGACTC
TCCAGCATCCTTGGAAGTGGCCTCTTCACAGTCATCTGAGAACCTAGAGG
AAGATGCTGTTTACAAGACCCACGTGGAGAAGGACTTCATTGCTTTCTGC
TCTTCAACGCCACACAACGTGTCCTGGAGAGACAGCACAATGGGCTCTA
TCTTCATCACACAACTCATCACATGCTTCCAGAAATATTCTTGGTGCTGC
CACCTAGAGGAAGTATTTCGGAAGGTACAGCAATCATTTGAAACTCCAA
GGGCCAAAGCTCAAATGCCCACCATAGAACGACTGTCCATGACAAGATA
TTTCTACCTCTTTCCTGGCAATTGAAAATGGAAGCCACAAGCAGCCCAGC
CCTCCTTAATCAACTTCAAGGAGCACCTTCATTAGTACAGCTTGCATATT
TAACATTTTGTATTTCAATAAAAGTGAAGACAAACGA
SEQ ID NO: 7:
Human Gasdermin D (Also known as: DF5L, DFNA5L, FKSG10,
GSDMDC1) Protein-484 a.a., Gasdermin D [Homo sapiens],
GenBank: AAH08904.1 (also NP_001159709)
MGSAFERVVRRVVQELDHGGEFIPVTSLQSSTGFQPYCLVVRKPSSSWFWK
PRYKCVNLSIKDILEPDAAEPDVQRGRSFHFYDAMDGQIQGSVELAAPGQA
KIAGGAAVSDSSSTSMNVYSLSVDPNTWQTLLHERHLRQPEHKVLQQLRSR
GDNVYVVTEVLQTQKEVEVTRTHKREGSGRFSLPGATCLQGEGQGHLSQK
KTVTIPSGSTLAFRVAQLVIDSDLDVLLFPDKKQRTFQPPATGHKRSTSEGA
WPQLPSGLSMMRCLHNFLTDGVPAEGAFTEDFQGLRAEVETISKELELLDRE
LCQLLLEGLEGVLRDQLALRALEEALEQGQSLGPVEPLDGPAGAVLECLVL
SSGMEVPELAIPVVYLLGALTMESETQHKELAEALESQTLLGPLELVGSLLE
QSAPWQERSTMSLPPGLLGNSWGEGAPAWVLLDECGLELCiEDTPHVCWEP
QAQGRMCALYASLALLSGLSQEPH
SEQ ID NO: 8:
Human Gasdermin D (Also known as: DF5L, DFNA5L, FKSG10,
GSDMDC1) mRNA/nucleotide- GenBank Accession numbers include
XM_011517301.2, NM_001166237.1, NM_024736.6, BC069000.1,
and BC008904.2.; Homo sapiens gasdermin D, mRNA
(cDNA clone MGC: 15043 IMAGE: 3634992), complete cds,
GenBank: BC008904.2, 1823 bp
CCTGGGCGGGCCCTGCGTCAGGTTGCAGTTTCACTTTTAGCTCTGGGCA
CCTCCAGCTCCTGCTCGCCGGACGGCTCCCAGGGAGAGCAGACGCGCC
AGACGCGCCACCCTCGGGGCGCCGACGGTCACGGAGCATGGGGTCGG
CCTTTGAGCGGGTAGTCCGGAGAGTGGTCCAGGAGCTGGACCATGGTG
GGGAGTTCATCCCTGTGACCAGCCTGCAGAGCTCCACTGGCTTCCAGC
CCTACTGCCTGGTGGTTAGGAAGCCCTCAAGCTCATGGTTCTGGAAAC
CCCGTTATAAGTGTGTCAACCTGTCTATCAAGGACATCCTGGAGCCGG
ATGCCGCGGAACCAGACGTGCAGCGTGGCAGGAGCTTCCACTTCTACG
ATGCCATGGATGGGCAGATACAGGGCAGCGTGGAGCTGGCAGCCCCA
GGACAGGCAAAGATCGCAGGCGGGGCCGCGGTGTCTGACAGCTCCAG
CACCTCAATGAATGTGTACTCGCTGAGTGTGGACCCTAACACCTGGCA
GACTCTGCTCCATGAGAGGCACCTGCGGCAGCCAGAACACAAAGTCCT
GCAGCAGCTGCGCAGCCGCGGGGACAACGTGTACGTGGTGACTGAGG
TGCTACAGACACAGAAGGAGGTGGAAGTCACGCGCACCCACAAGCGG
GAGGGCTCGGGCCGGTTTTCCCTGCCCGGAGCCACGTGCTTGCAGGGT
GAGGGCCAGGGCCATCTGAGCCAGAAGAAGACGGTCACCATCCCCTC
AGGCAGCACCCTCGCATTCCGGGTGGCCCAGCTGGTTATTGACTCTGA
CTTGGACGTCCTTCTCTTCCCGGATAAGAAGCAGAGGACCTTCCAGCC
ACCCGCGACAGGCCACAAGCGTTCCACGAGCGAAGGCGCCTGGCCAC
AGCTGCCCTCTGGCCTCTCCATGATGAGGTGCCTCCACAACTTCCTGAC
AGATGGGGTCCCTGCGGAGGGGGCGTTCACTGAAGACTTCCAGGGCCT
ACGGGCAGAGGTGGAGACCATCTCCAAGGAACTGGAGCTTTTGGACA
GAGAGCTGTGCCAGCTGCTGCTGGAGGGCCTGGAGGGGGTGCTGCGG
GACCAGCTGGCCCTGCGAGCCTTGGAGGAGGCGCTGGAGCAGGGCCA
GAGCCTTGGGCCGGTGGAGCCCCTGGACGGTCCAGCAGGTGCTGTCCT
GGAGTGCCTGGTGTTGTCCTCCGGAATGCTGGTGCCGGAACTCGCTAT
CCCTGTTGTCTACCTGCTGGGGGCACTGACCATGCTGAGTGAAACGCA
GCACAAGCTGCTGGCGGAGGCGCTGGAGTCGCAGACCCTGTTGGGGC
CGCTCGAGCTGGTGGGCAGCCTCTTGGAGCAGAGTGCCCCGTGGCAGG
AGCGCAGCACCATGTCCCTGCCCCCCGGGCTCCTGGGGAACAGCTGGG
GCGAAGGAGCACCGGCCTGGGTCTTGCTGGACGAGTGTGGCCTAGAG
CTGGGGGAGGACACTCCCCACGTGTGCTGGGAGCCGCAGGCCCAGGG
CCGCATGTGTGCACTCTACGCCTCCCTGGCACTGCTATCAGGACTGAG
CCAGGAGCCCCACTAGCCTGTGCCCGGGCATGGCCTGGCAGCTCTCCA
GCAGGGCAGAGTGTTTGCCCACCAGCTGCTAGCCCTAGGAAGGCCAG
GAGCCCAGTAGCCATGTGGCCAGTCTACCATGGGGCCCAGGAGTTGGG
GAAACACAATAAAGGTGGCATACGAAGGAAAAAAAAAAAAAAAAAA
AAACCAAAAAAAAAAAAAAAAAAAAAAAAAAAAAAAAAAAAAAAA
AAAAAAAAAAAAAAAA
SEQ ID NO: 9:
siRNA directed against caspase-4, S1
5′-GUGUAGAUGUAGAAGAGAATT-3′
SEQ ID NO: 10:
siRNA directed against caspase-4, S2
5′-CCUAGAGGAAGAUGCUGUUTT-3′
SEQ ID NO: 11:
siRNA directed against caspase-4
5′-CUACACUGUGGUUGACGAA-3′
SEQ ID NO: 12
siRNA directed against caspase-4
5′-CCAUAGAACGAGCAACCUU-3′
SEQ ID NO: 13:
siRNA directed against caspase-4
5′-CAGCAGAAUCUACAAAUAU-3′
SEQ ID NO: 14:
siRNA directed against caspase-4
5′-CGGAUGUGCUGCUUUAUGA-3′
SEQ ID NO: 15:
shRNA against cGAS
CCGGCCTGCTGTAACACTTCTTATTCTCGAGAATAAGAAGTGTTACAG
CAGGTTTTTTG
SEQ ID NO: 16:
shRNA against Caspase-4
CCGGAGACTATGTAAAGAAAGAGCTCTCGAGAGCTCTTTCTTTACATA
GTCTTTTTT
SEQ ID NO: 17:
shRNA against STING
CCGGCCAACATTCGCTTCCTGGATACTCGAGTATCCAGGAAGCGAATG
TTGGTTTTTTG
SEQ ID NO: 18:
shRNA against PPIF
CCGGCTGTGGCCAGTTGAGCTAATCCTCGAGGATTAGCTCAACTGGCC
ACAGTTTTTG
SEQ ID NO: 19:
shRNA against GSDMD
CCGGCAACCTGTCTATCAAGGACATCTCGAGATGTCCTTGATAGACAG
GTTGTTTTTTG
SEQ ID NO: 20:
shRNA against IFNB
CCGGCAGAGTGGAAATCCTAAGGAACTCGAGTTCCTTAGGATTTCCAC
TCTGTTTTT
SEQ ID NO: 21:
shRNA against IFNAR1
CCGGGCCAAGATTCAGGAAATTATTCTCGAGAATAATTTCCTGAATCTTG
GCTTTTTG

### Examples

### Results

### Caspase-4 is Activated in AMD

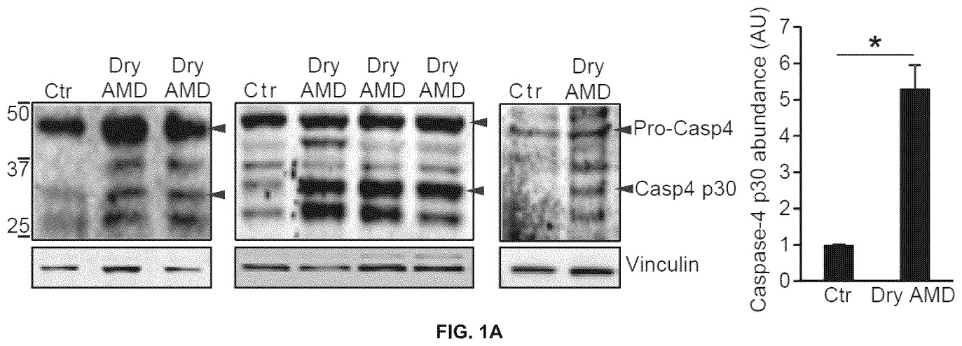

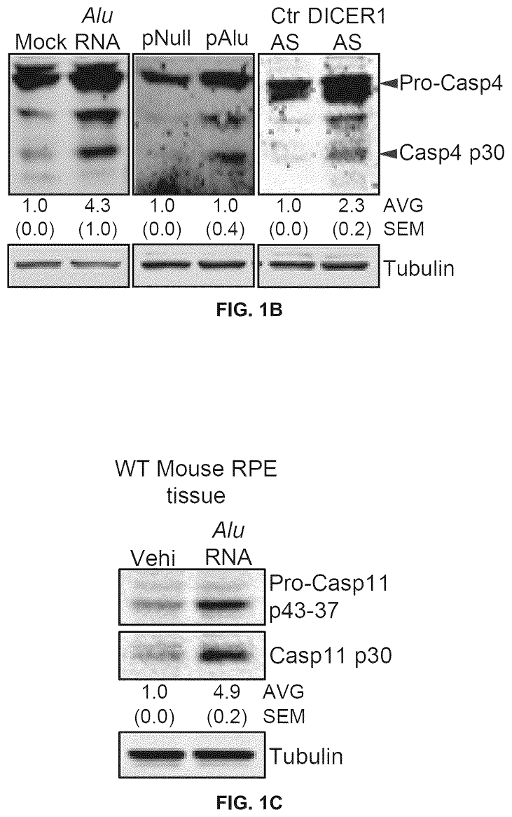

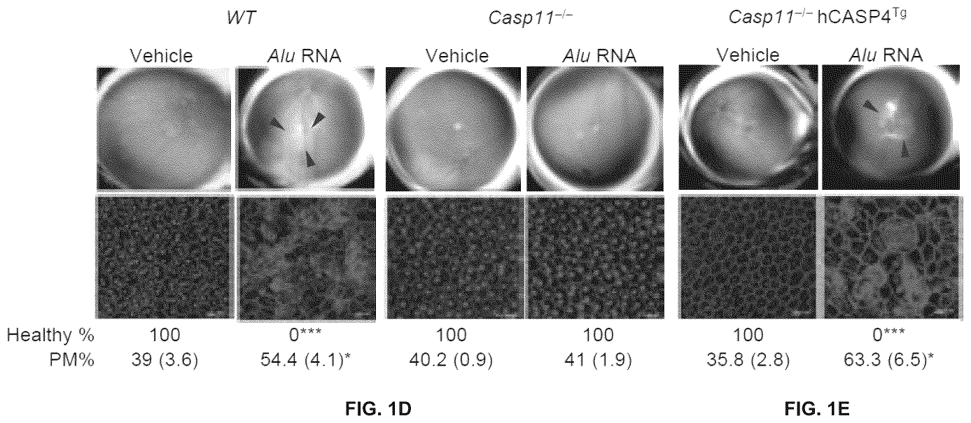

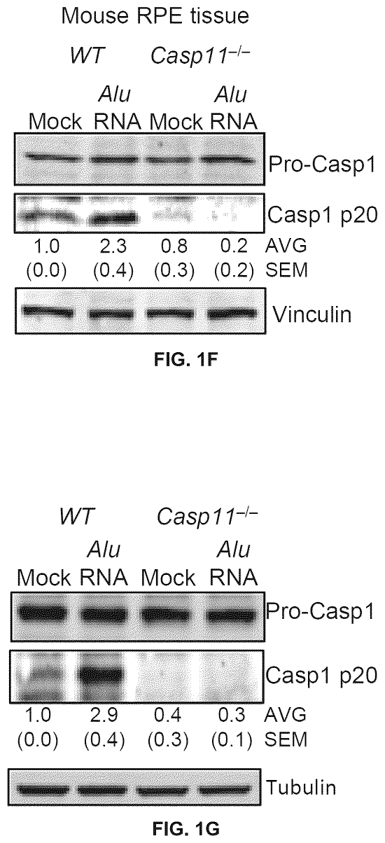

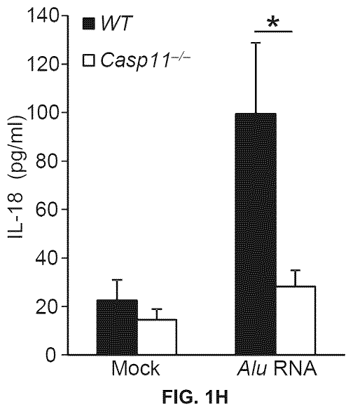

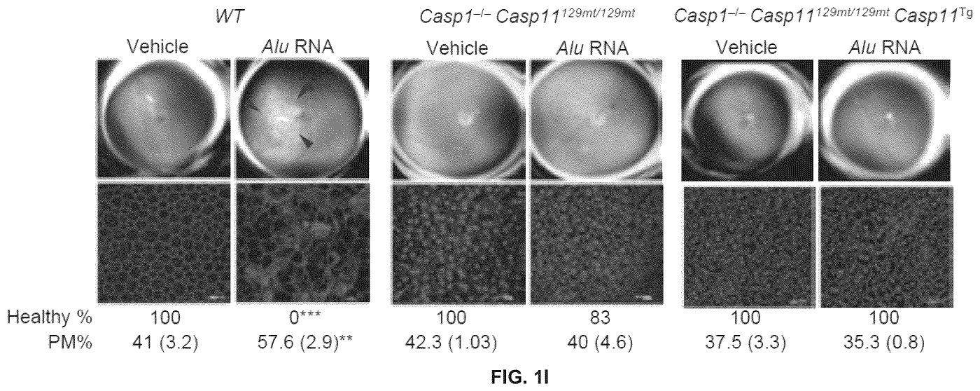

Caspase-4 (a human protein—also known as caspase-11 in mouse), which governs non-canonical inflammasome activation, was recently implicated in the immune response to exogenous pathogen-associated molecular patterns (PAMPs) such as intracellular LPS¹¹⁻¹⁷ and endogenously produced oxidized phospholipids (oxPAPC)¹¹⁻¹⁸. Caspase-4 abundance in the RPE and choroid of human geographic atrophy eyes is significantly increased compared to aged normal human eyes, as monitored by western blotting (FIG. 1A and Supplementary FIG. 1i). Introduction of in vitro transcribed Alu RNA or plasmid-mediated enforced expression of Alu RNA (pAlu) induced and activated caspase-4 in primary human RPE cells (FIG. 1B and Supplementary FIGS. 1A and 1C). Anti-sense oligonucleotide-mediated knockdown of DICER1 similarly induced caspase-4 activation in human RPE cells (FIG. 1i), which was blocked by concomitant anti-sense mediated inhibition of Alu RNA (Supplementary FIG. 1D). Caspase-11 activation was induced by subretinal injection of Alu RNA in wild-type (WT) C57BL/6J mice (FIG. 1C), and by Alu RNA transfection in primary RPE cells isolated from WT mice (Supplementary FIG. 1E). Collectively, these data identify caspase-4 as being preferentially activated in human AMD, and dysregulation of DICER1 and Alu RNA as novel endogenous agonists of caspase-4.

### Caspase-4 is Required for Alu RNA-Induced RPE Degeneration and Inflammasome Activation

RPE degeneration was quantified based on zonula occludens (ZO)-1-stained flat mount images using two strategies (Supplementary Text):

1. Binary assignment (healthy versus unhealthy)¹⁹⁻²¹ by two masked raters (inter-rater agreement=98.6%; Pearson r²=0.95, P<0.0001; Fleiss κ=0.97, P<0.0001).

2. Semi-automated cellular morphometry analysis by three masked raters adapting our prior analysis of the planar architecture of the corneal endothelium²², which resembles the RPE in its polygonal tessellation. Inter-rater agreement was high for all three metrics (multiple adjusted r²=0.99 (cell size), 0.72 (polymegethism, i.e., coefficient of variation of cell size), 0.99 (cell density)).

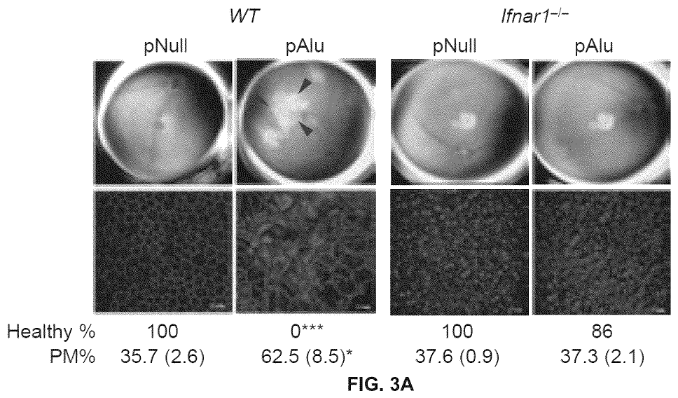

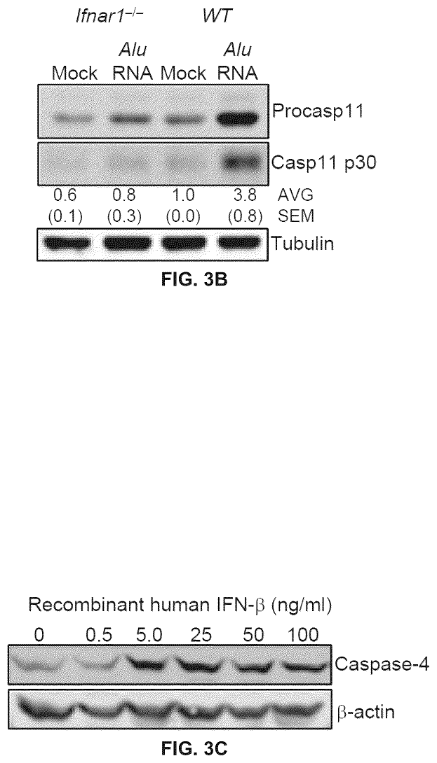

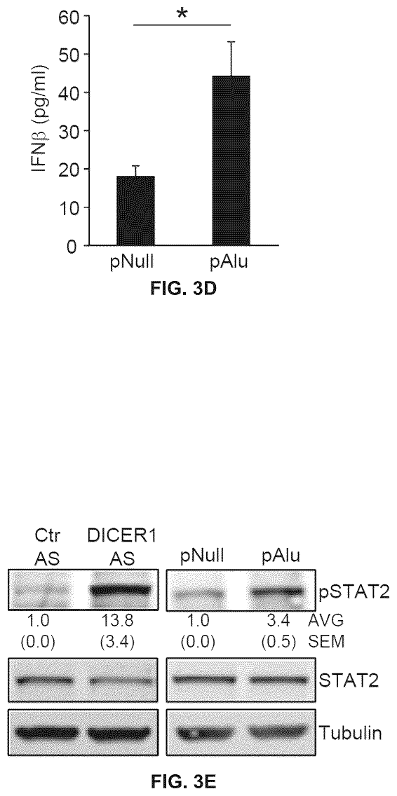

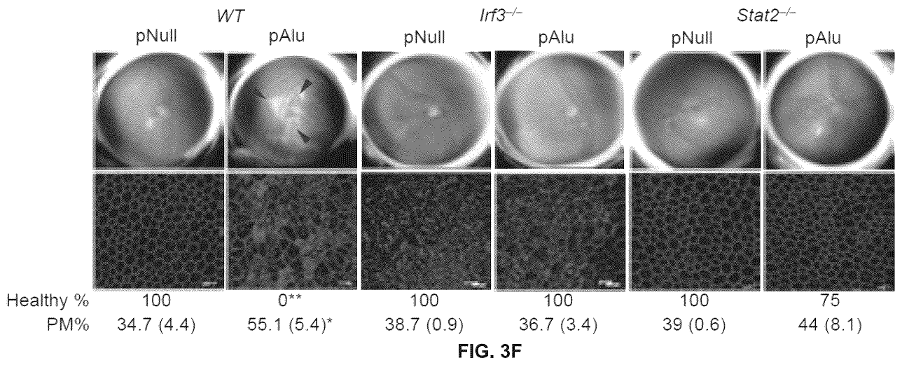

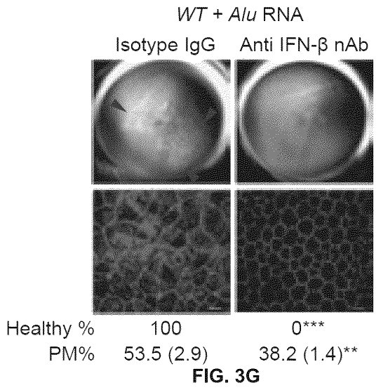

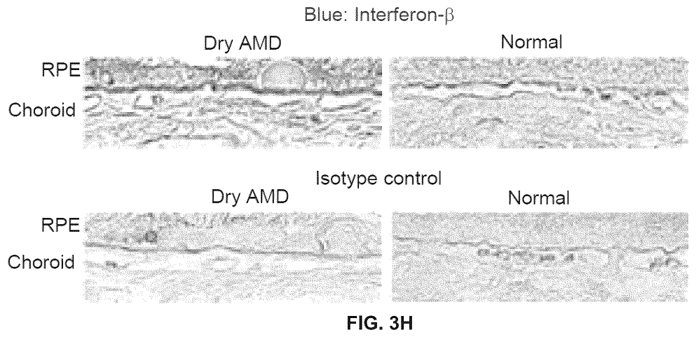

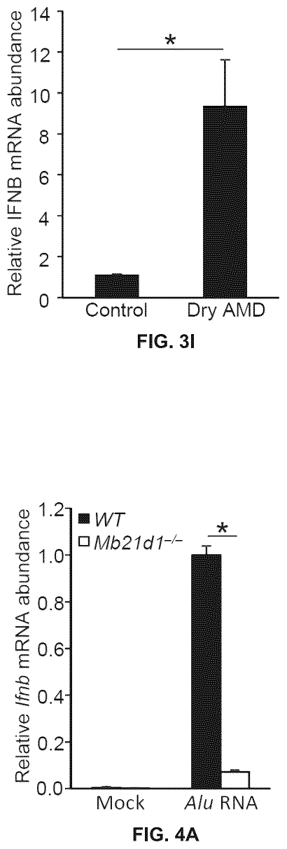

For eyes treated with Alu RNA, pAlu, and their respective controls, inter-rater agreement on binary assignment was 100%, and the fraction of eyes classified as healthy was 100% for both control groups versus 0% for Alu RNA or pAlu treatments (P<0.0001 for both comparisons, Fisher exact test). All three morphometric features were significantly different between control treatments versus Alu RNA or pAlu treatments (P<0.0001, t test; Supplementary FIG. 3). Given the similarity among all three features in differentiating healthy versus degenerated RPE cells, for all remaining groups, we quantified polymegethism, a prominent geometric feature of RPE cells in human geographic atrophy^{3,23-25}.

Exogenous delivery or endogenous over-expression of Alu RNA induces RPE degeneration in WT mice (FIG. 1D and Supplementary FIG. 1F). In contrast, neither subretinal injection of Alu RNA nor pAlu induced RPE degeneration in Casp11^{-/-} mice (FIG. 1D and Supplementary FIG. 1F). 129S6 mice, which lack functional caspase-11 due to a passenger mutation¹⁴, also were resistant to RPE degeneration induced by Alu RNA or pAlu (Supplementary FIG. 1G). Subretinal delivery of a cell-permeable, non-immunogenic 17+2-nt cholesterol-conjugated siRNA^{26,27} targeting Dicer1 induced RPE degeneration in WT but not 129S6 mice (Supplementary FIG. 1H). Transgenic expression of human caspase-4 on the caspase-11 deficient background (Casp11^{-/-} hCASP4^{TG})²⁸ restored the susceptibility of these mice to Alu RNA-induced RPE degeneration (FIG. 1E), demonstrating that human caspase-4 can compensate for mouse caspase-11 in this system. Collectively, these data demonstrate the critical role of caspase-4 (or mouse caspase-11) in responding to pathological accumulation of endogenous Alu mobile element transcripts.

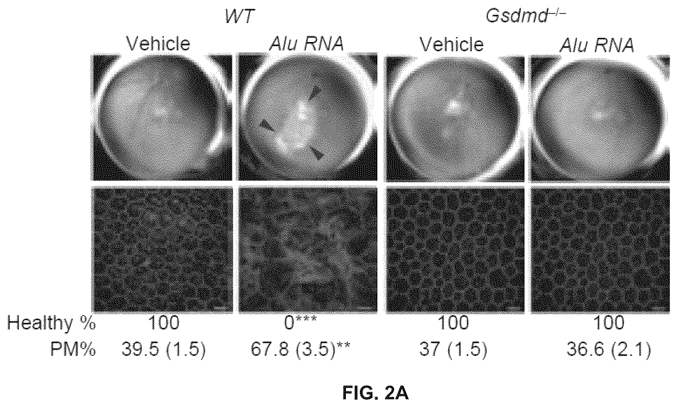

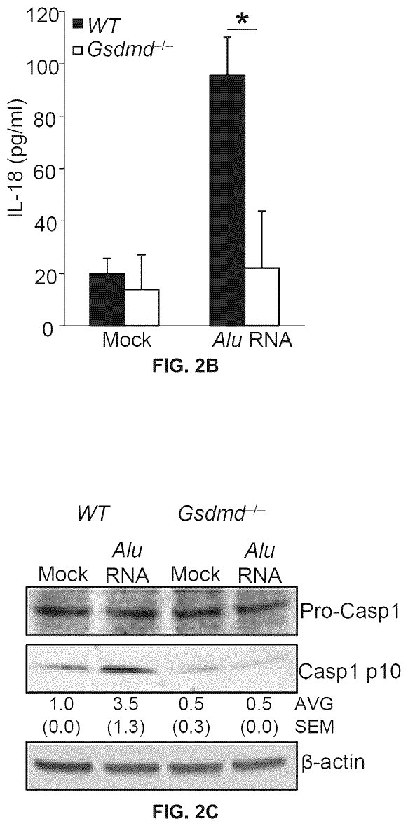

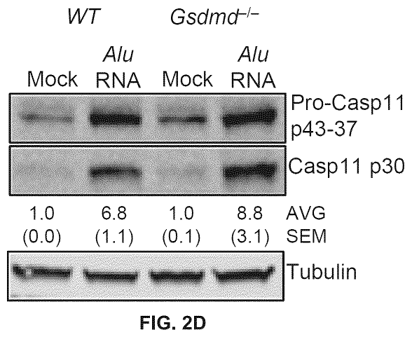

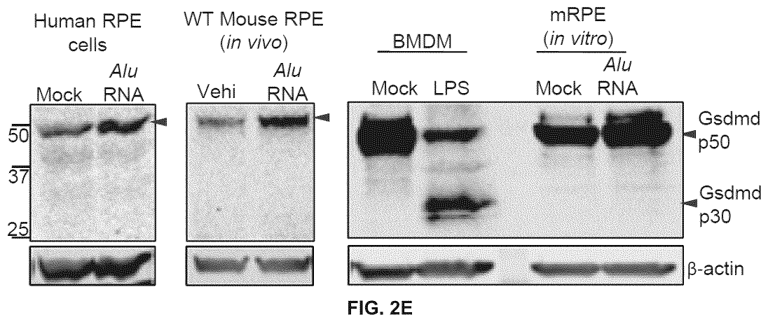

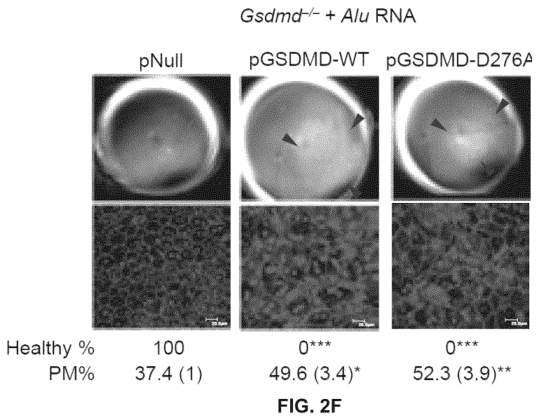

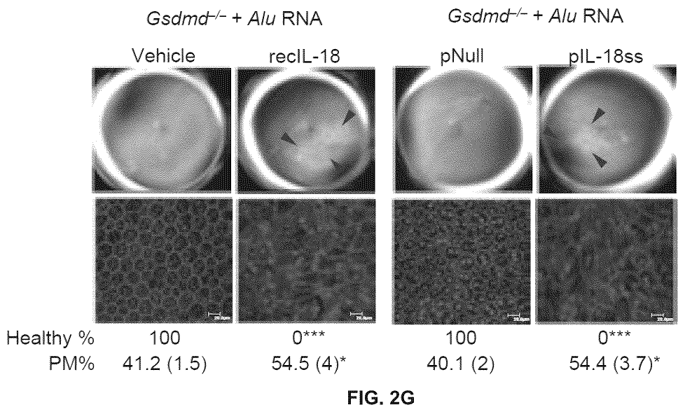

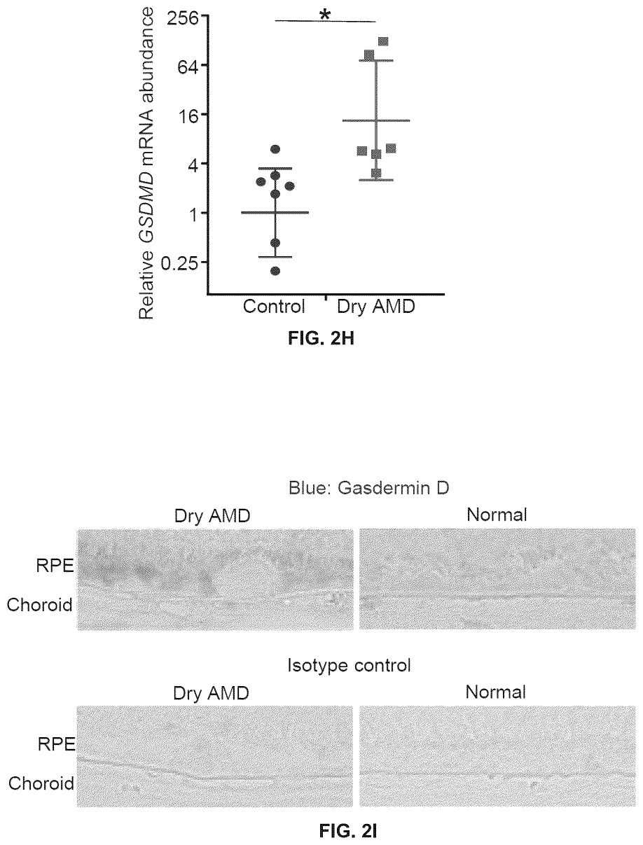

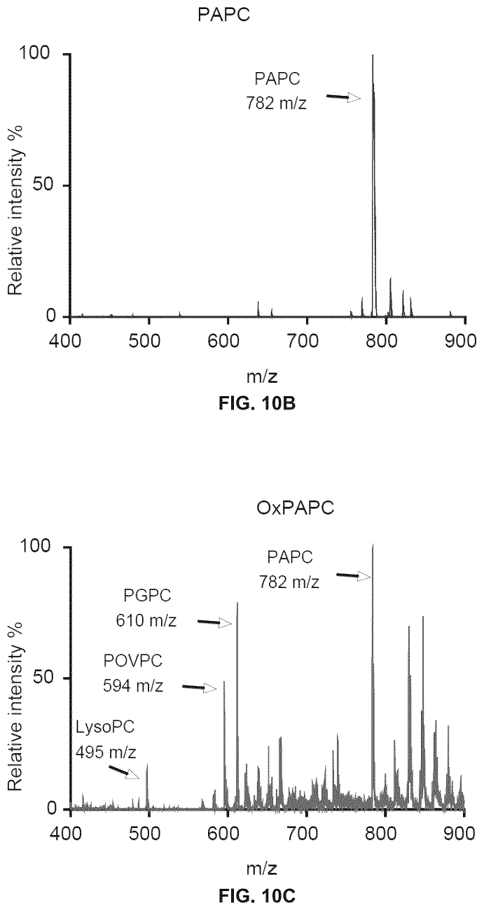

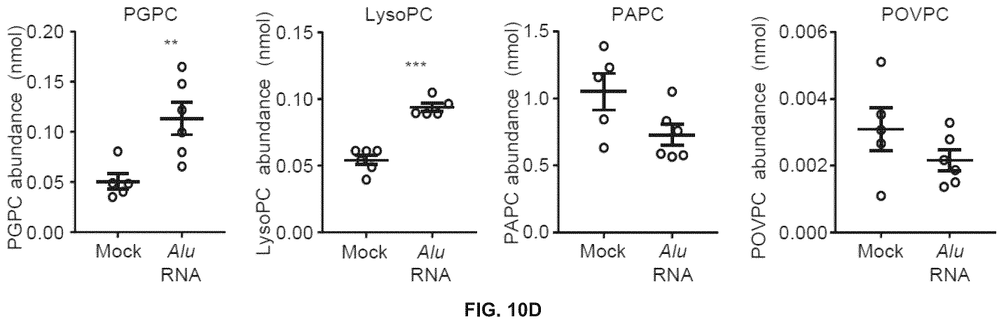

Previously we reported that Alu RNA does not induce RPE degeneration in caspase-1 deficient mice². However, this Casp1^{-/-} strain was subsequently reported to also lack functional caspase-11 as a result of a passenger mutation in their 129S6 background¹⁴; thus, they are properly referred to as Casp1^{-/-}1 Casp11^{129mt/129mt} mice. We sought to clarify the molecular hierarchy of caspases-1 and 11 in response to Alu RNA. Whereas Alu RNA treatment induced caspase-1 activation in WT systems, Alu RNA failed to stimulate caspase-1 activation in Casp11 mice (FIG. 1F), or in primary RPE cells isolated from Casp11^{-/-} mice (FIG. 1G). Reconstitution of caspase-11 into Casp11^{-/-} mouse RPE cells restored caspase-1 activation by Alu RNA (Supplementary FIGS. 2A and 2B). Alu RNA failed to induce IL-18 secretion in Casp11^{-/-} mouse RPE cells (FIG. 1H). In contrast, caspase-11 was dispensable for IL-18 secretion induced by the canonical inflammasome agonist monosodium urate (MSU) crystals (Supplementary FIG. 10D).

Alu RNA did not induce RPE degeneration in Casp1^{-/-} Casp11^{129mt/129mt} Casp11^{Tg} mice¹⁴, in which mouse caspase-11 was functionally reconstituted by a bacterial artificial chromosome transgene (FIG. 1I and Supplementary FIG. 2C). Additionally, as previously observed², Casp1^{-/-} Casp11^{129mt/129mt} mice were not susceptible to Alu-induced RPE degeneration (FIG. 1I and Supplementary FIG. 2C), suggesting that both caspase-4/11 and caspase-1 are required for Alu toxicity.

Previously we demonstrated that PYCARD, an adaptor protein involved in inflammasome activation, and the purinoceptor P2X7 (encoded by P2rx7) were required for Alu toxicity^{2,9,20}. We assessed whether PYCARD and P2X7 were also required for Alu RNA-induced caspase-11 activation. Alu RNA-induced activation of caspase-11 was reduced in P2rx7 but not Pycard^{-/-} mouse RPE cells, suggesting that caspase-11 is mechanistically positioned between these signaling molecules (Supplementary FIGS. 2D and 2E). Collectively these findings support a model of non-canonical inflammasome activation by Alu RNA wherein caspase-4/11 lies upstream of caspase-1 activation²⁹.

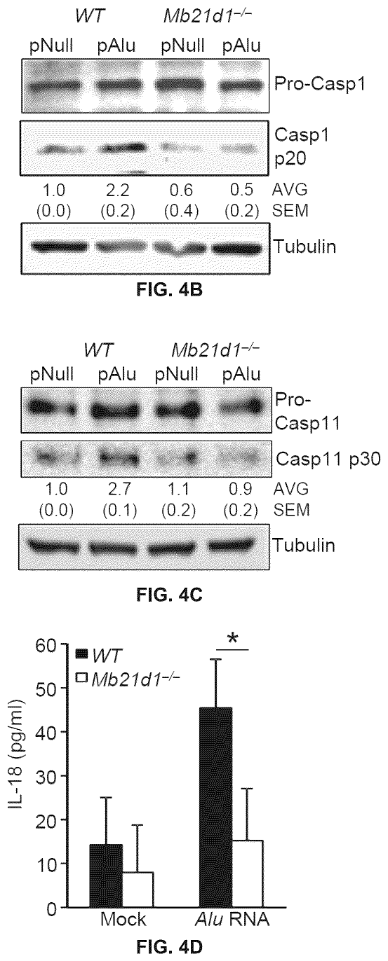

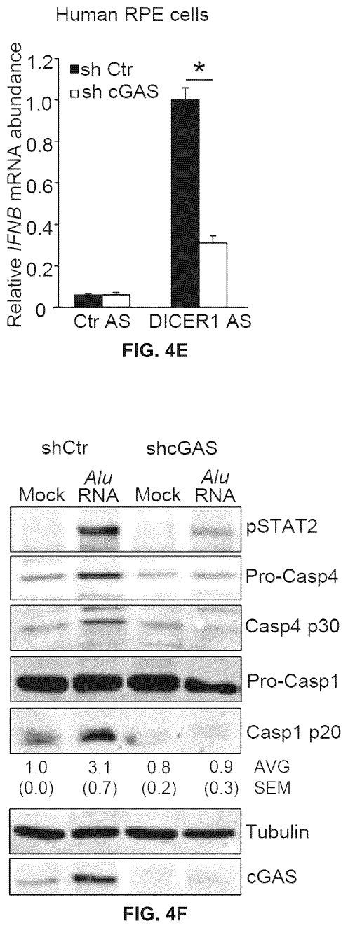

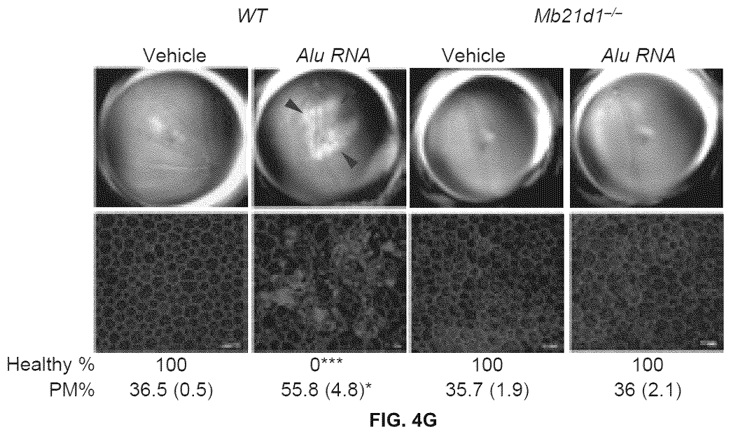

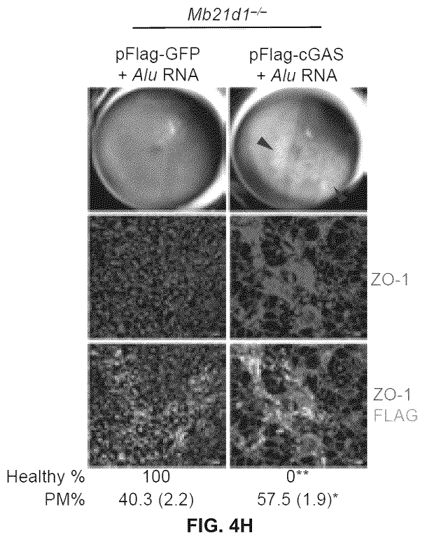

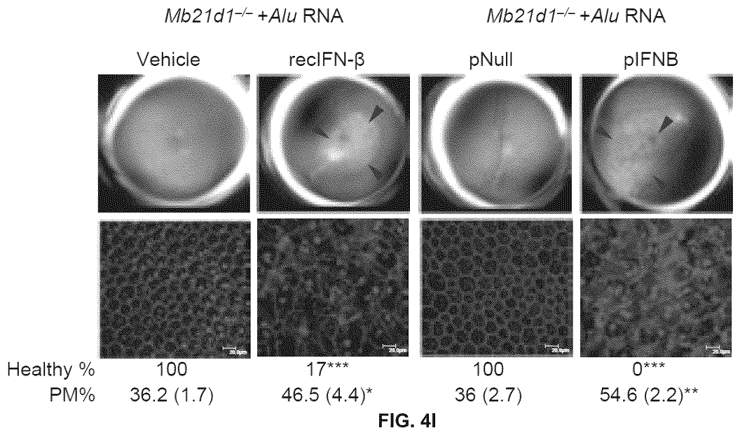

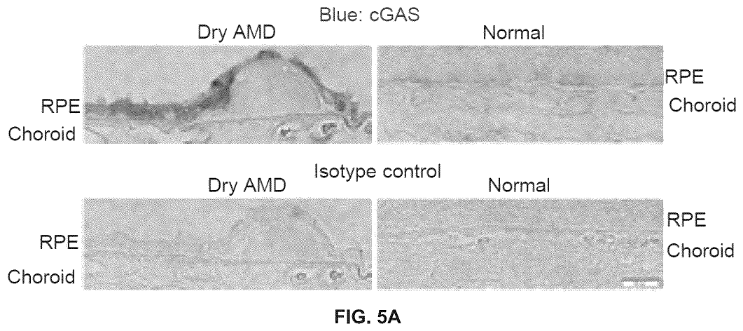

Recent studies implicated the endogenous lipid molecule, oxidized phospholipid oxPAPC (oxidized 1-palmitoyl-2-arachidonyl-sn-glycero-3-phosphocholine) in caspase-11-mediated non-canonical NLRP3 inflammasome activation¹⁸. To test whether Alu RNA promotes accumulation of these endogenous ligands, we extracted lipids from Alu RNA-treated human RPE cells and used liquid chromatography-mass spectrometry to quantify the following products of 1-palmitoyl-2-arachidonoyl-3-phosphatidylcholine (PAPC): 1-palmitoyl-2-glutaryl-3-phosphatidylcholine (PGPC), 1-palmitoyl-2-(5-oxovaleryl)-3-phosphatidylcholine (POVPC), and 1-palmitoyl-2-hydroxy-3-phosphatidylcholine (LysoPC) (Supplementary FIGS. 4A-4C). Compared to control cells, Alu RNA-treated human RPE cells exhibited a two-fold increase in oxPAPC-PGPC and LysoPC levels (Supplementary FIG. 4D), concomitant with a trend towards a reduction in precursor PAPC (Supplementary FIG. 4D). These results suggest an indirect mechanism of Alu-driven caspase-11 engagement possibly via oxidized phospholipid-derived damage-associated molecular patterns (DAMPs).

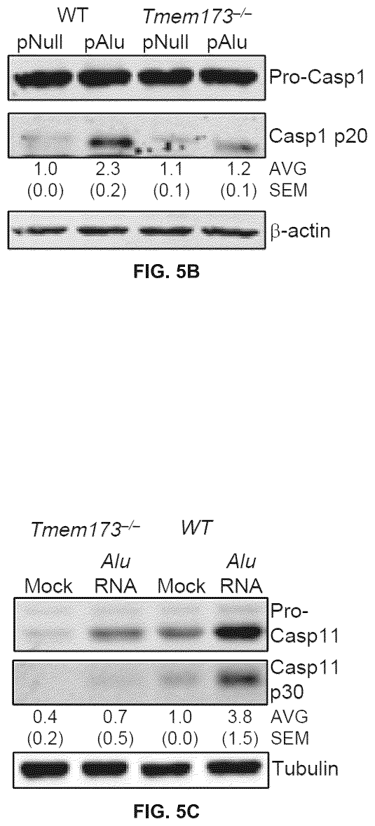

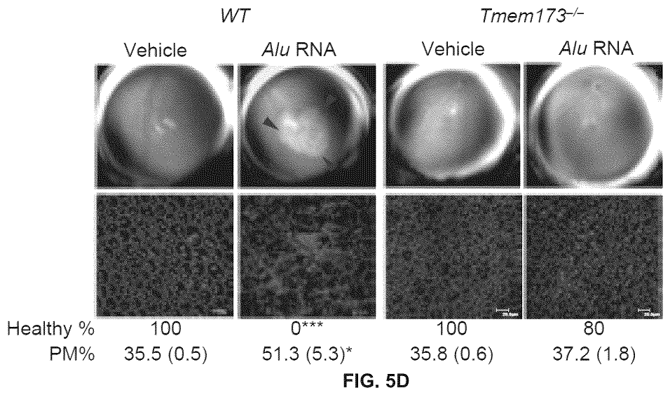

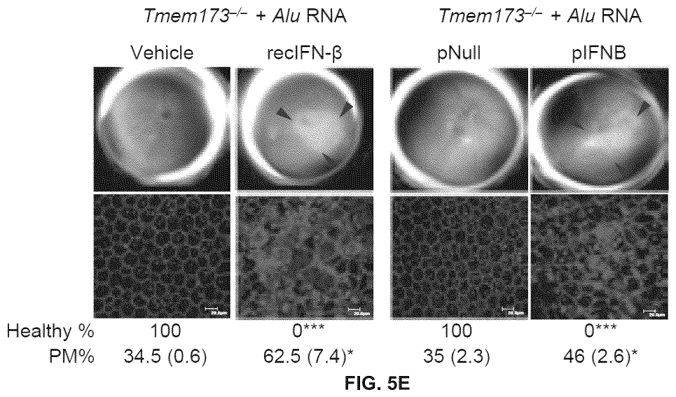

Gasdermin D is Required for Alu RNA-Induced RPE Degeneration and Inflammasome Activation Caspase-11- and caspase-1-dependent pyroptotic cell death can be executed by a pore-forming protein, gasdermin D (encoded by Gsdmd)³⁰⁻³³. Gsdmd^{-/-} mice were resistant to Alu RNA-induced RPE degeneration (FIG. 2A and Supplementary FIGS. 5A and 5B). Consistent with the role of gasdermin D in non-canonical inflammasome activation by intracellular LPS³¹, Alu RNA-induced caspase-1 activation and IL-18 secretion were reduced in Gsdmd^{-/-} mouse RPE cells (FIGS. 2B and 2C). However, caspase-11 activation in Gsdmd^{-/-} mice was not impaired (FIG. 2D), suggesting that loss of caspase-1 activation in gasdermin D is not due an indirect effect of gasdermin D on caspase-11, and that caspase-11 lies mechanistically upstream of gasdermin D.

Execution of pyroptosis by gasdermin D requires its cleavage into a pore-forming p30 fragment³²⁻³⁵. Interestingly, although gasdermin D is required for Alu RNA-induced RPE degeneration and IL-18 secretion, we did not observe its cleavage into a p30 fragment in RPE cells either in cell culture or in vivo (FIG. 2E); however, as reported earlier³¹, intracellular LPS induced p30 cleavage in mouse bone marrow derived macrophages (BMDMs; FIG. 2E).

Next, we directly tested whether gasdermin D p30 cleavage was dispensable for the toxicity of Alu RNA by reconstituting Gsdmd^{-/-} mice with WT gasdermin D (pGSDMD-WT) or mutant gasdermin D (pGSDMD-D276A), which is unable to undergo cleavage into the pyroptotic p30 fragment³¹. Interestingly, expression of either WT or the p30-cleavage incompetent mutant restored susceptibility to Alu RNA-induced RPE degeneration in Gsdmd^{-/-} mice, suggesting a non-pyroptotic function for gasdermin D in this system (FIG. 2F).

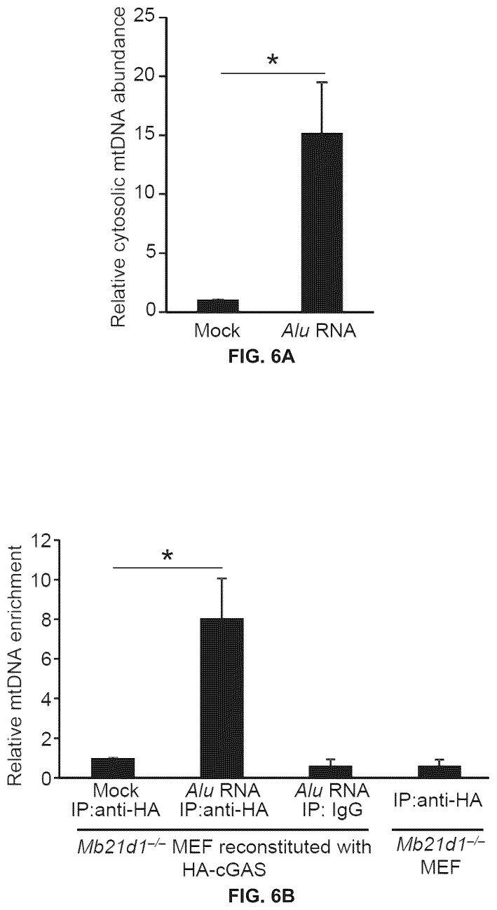

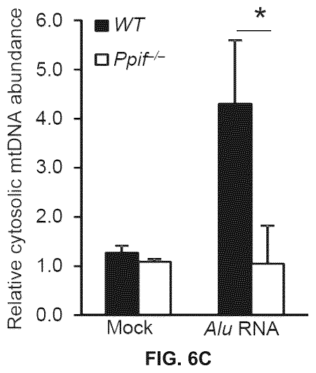

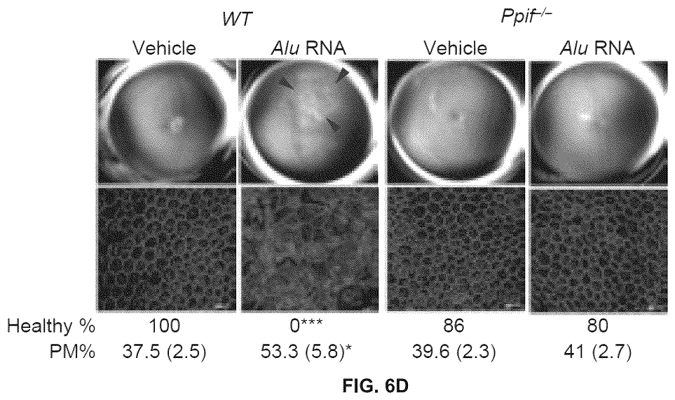

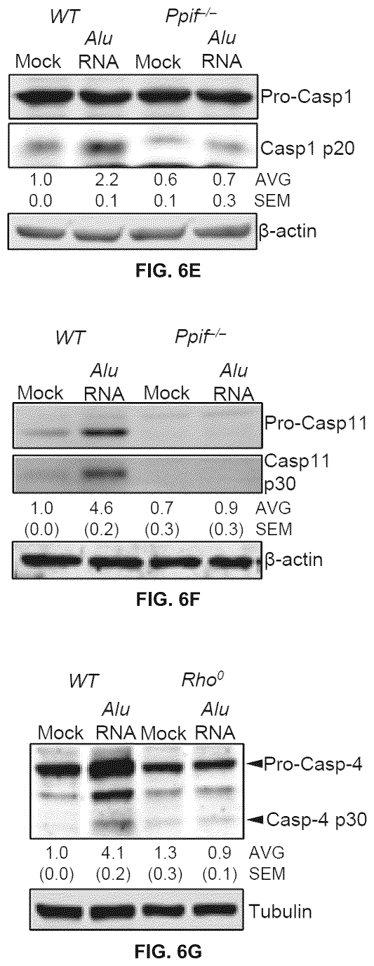

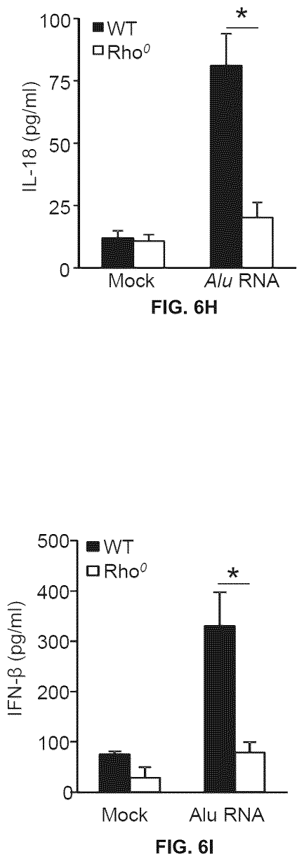

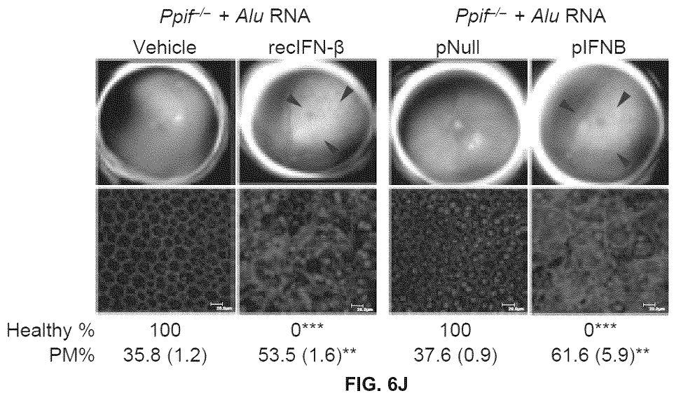

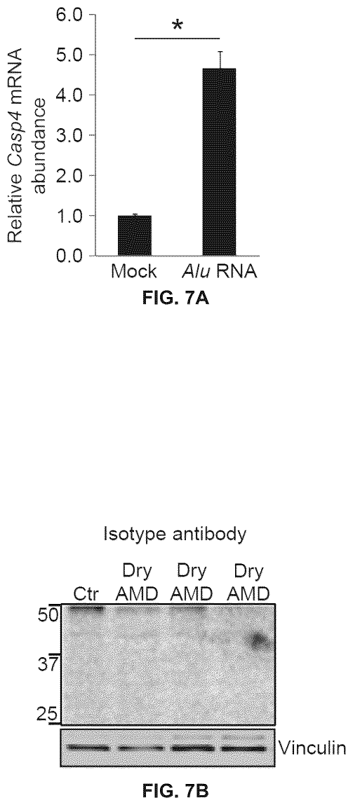

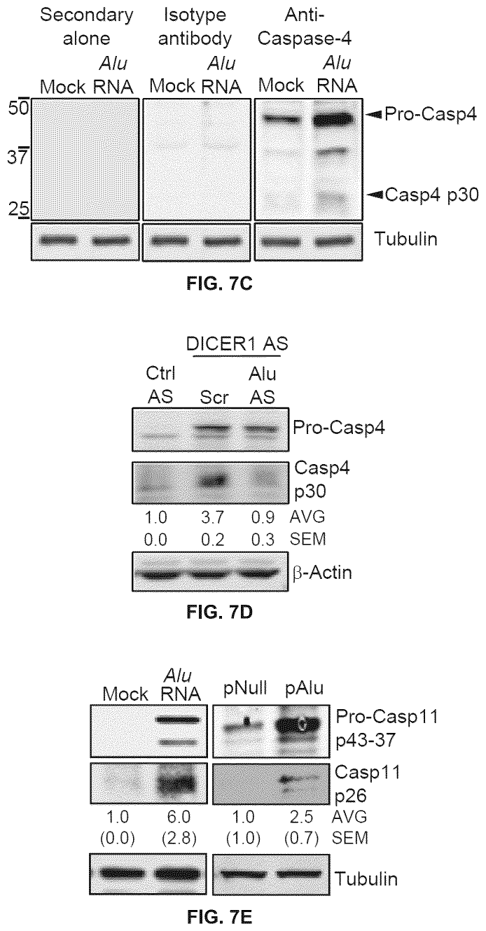

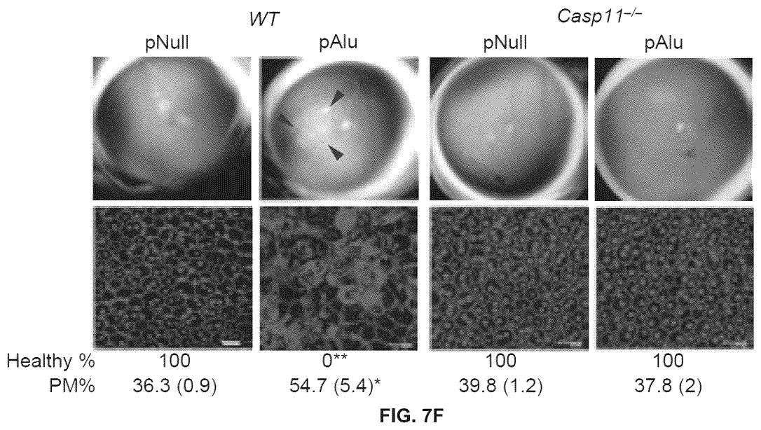

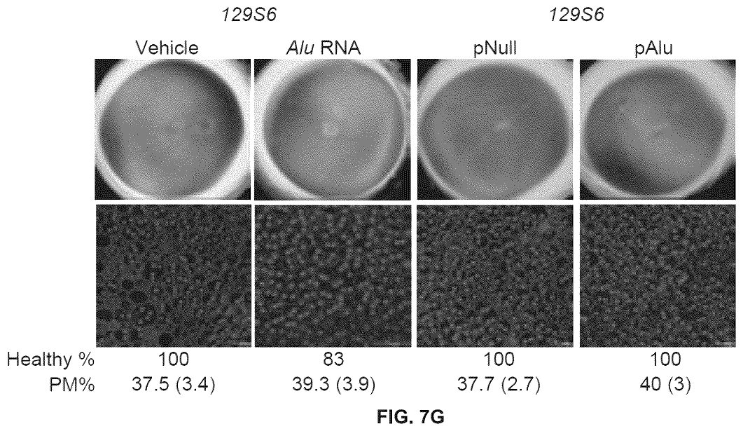

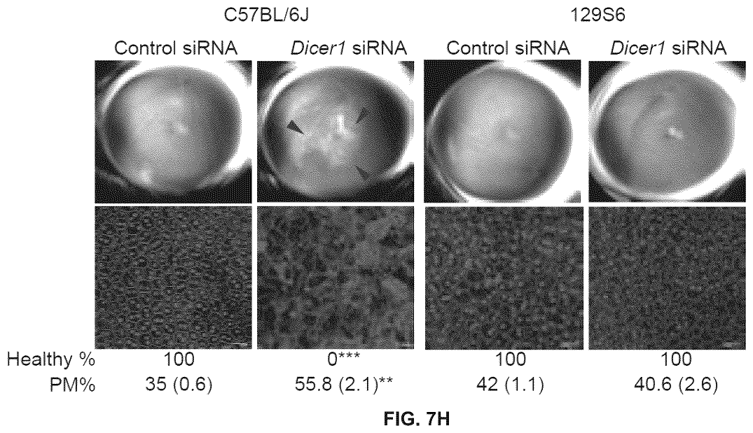

Previously we demonstrated that Alu RNA induced activation of caspase-3 (refs. 3,21) as well as caspase-8, Fas, and FasL²¹, and that this well-characterized pathway of apoptosis inducers was critical for its RPE toxicity. In addition, we and others have shown molecular evidence consistent with apoptosis in the RPE in human eyes with geographic atrophy^{3,36}. To further clarify the precise route of Alu RNA-induced cell death, we performed live-cell imaging of annexin-V and propidium iodide (PI) in primary human RPE cells. Cells treated with Alu RNA developed plasma membrane blebs and displayed an annexin-V⁺ PI⁻ staining pattern, findings that are consistent with early apoptosis. After several hours of annexin-V positivity, cells frequently swelled and became PI-positive, consistent with late apoptosis or secondary necrosis^{37,38} (Supplementary FIGS. 6A and 6B). In vivo studies recapitulated our cell culture findings: RPE flat mounts from Alu RNA-exposed WT mice displayed a prominence of annexin-V⁺ PI⁻ cell death (Supplementary FIG. 7A). Alu RNA stimulation of RPE cells induced cleavage of caspase-3 and poly(ADP-ribose) polymerase 1 (PARP-1; Supplementary FIG. 7B), further supporting an apoptotic cell death pathway. These findings, coupled with our earlier demonstration that neither necrostatin-1, an inhibitor of primary necrosis³⁹, nor glycine, an inhibitor of pyroptosis⁴⁰, blocks Alu RNA-induced RPE degeneration^{2,21}, suggest that Alu RNA promotes cell death primarily via apoptosis rather than pyroptosis or necrosis in RPE cells.

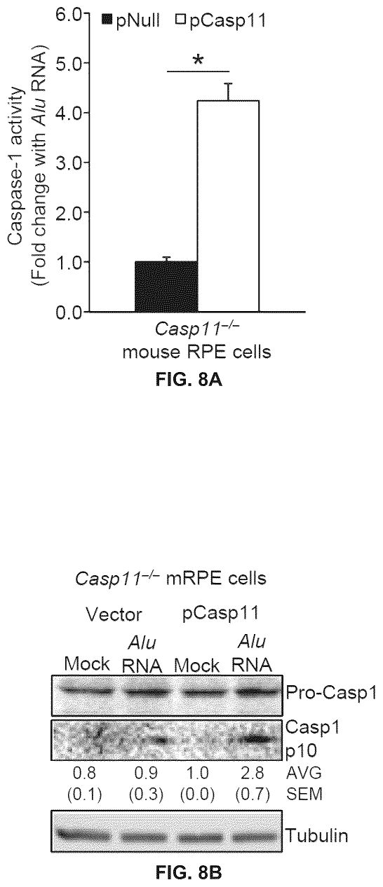

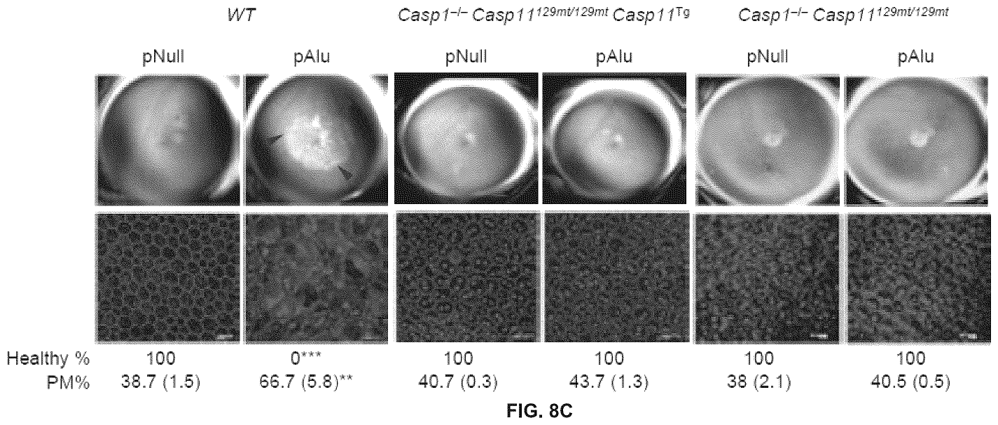

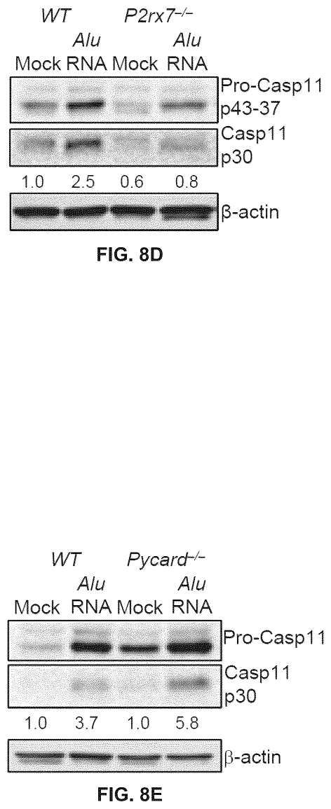

We further explored the inter-relationship of IL-18 and gasdermin D in Alu RNA-induced cell death. The resistance of Gsdmd^{-/-} mice to Alu RNA-induced RPE degeneration was overcome by recombinant mature IL-18 or mature TL-18 expression plasmid, suggesting that the absence of RPE degeneration in Gsdmd^{-/-} mice is due to loss of IL-18 secretion, and not due to lack of pyroptosis (FIG. 2G). Supportive of this concept, Alu RNA induced secretion of IL-18 in Gsdmd^{-/-} mouse RPE cells that were reconstituted with either pGSDMD-WT or the p30 cleavage-incompetent pGSDMD-D276A (Supplementary FIG. 5C). Additionally, whereas annexin-V⁺ cells were not visible in RPE flat mounts of Alu RNA-treated Gsdmd^{-/-} mice, administration of recombinant mature TL-18 led to the appearance of numerous annexin-V⁺ PI⁻ cells (Supplementary FIG. 8). Taken together with our earlier demonstration that IL-18 neutralization or IL-18 receptor ablation in mice with intact gasdermin D blocks Alu RNA toxicity in vivo², these findings suggest that gasdermin D is required for Alu RNA-induced inflammasome activation, and for RPE toxicity driven via IL-18-dependent apoptosis.

Gasdermin D mRNA abundance was elevated in the RPE of human geographic atrophy eyes compared to unaffected age-matched controls (FIG. 2H). In contrast, we observed similar levels of MIP-la, IL-8, and IL-6 in human geographic atrophy and normal specimens (Supplementary FIG. 5D), suggesting there is no global elevation of pro-inflammatory cytokines in GA, but rather a more specific increase in inflammasome pathway genes. We also observed increased gasdermin D immunolocalization in the RPE of human geographic atrophy eyes compared to unaffected age-matched controls (FIG. 2I).

### Alu RNA-Induced Non-Canonical Inflammasome Activation is Driven by Type I Interferon (IFN) Signaling

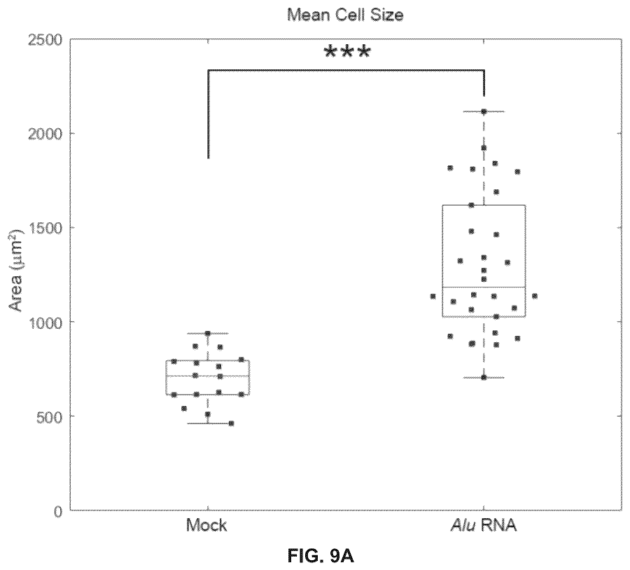

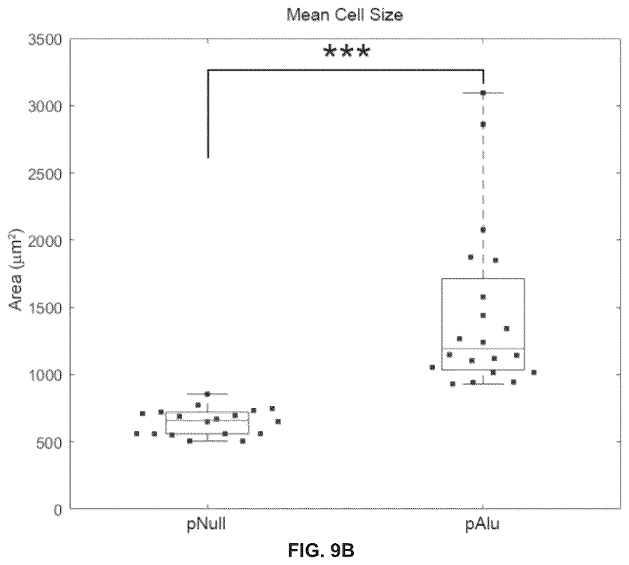

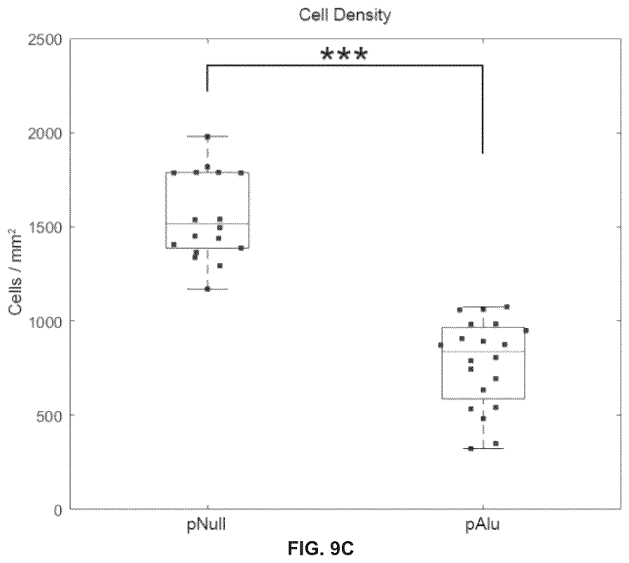

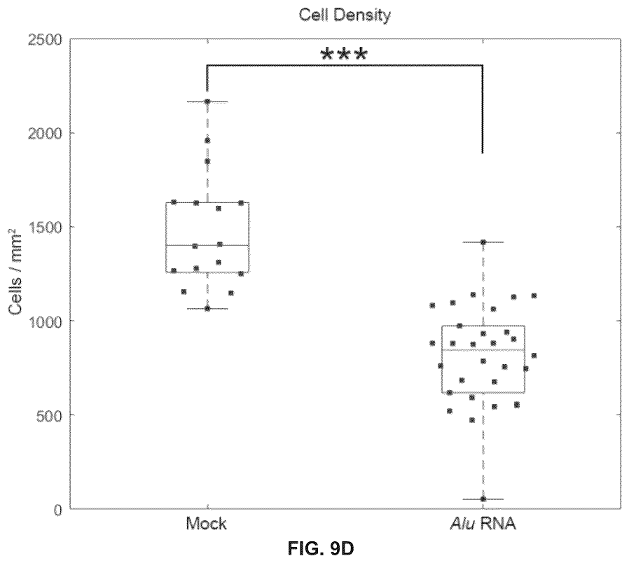

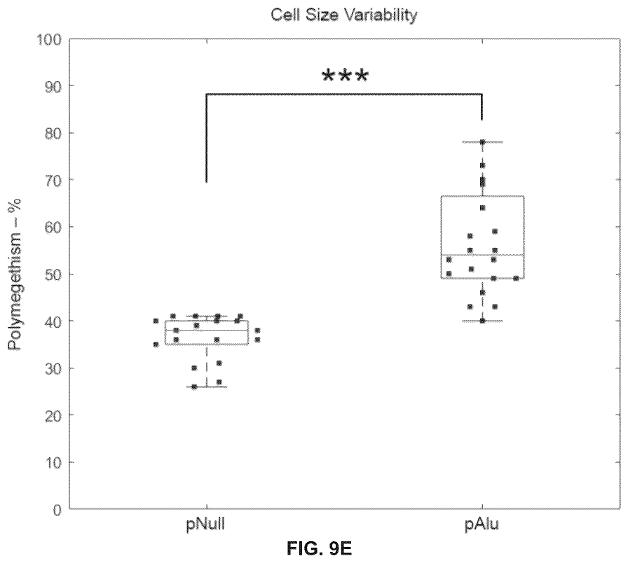

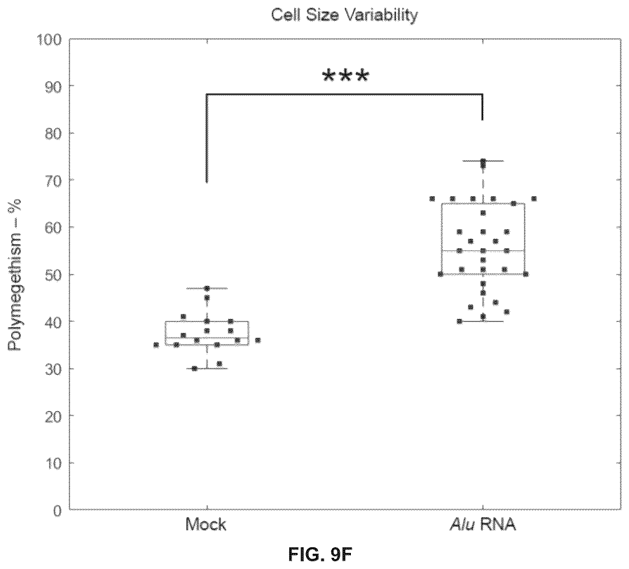

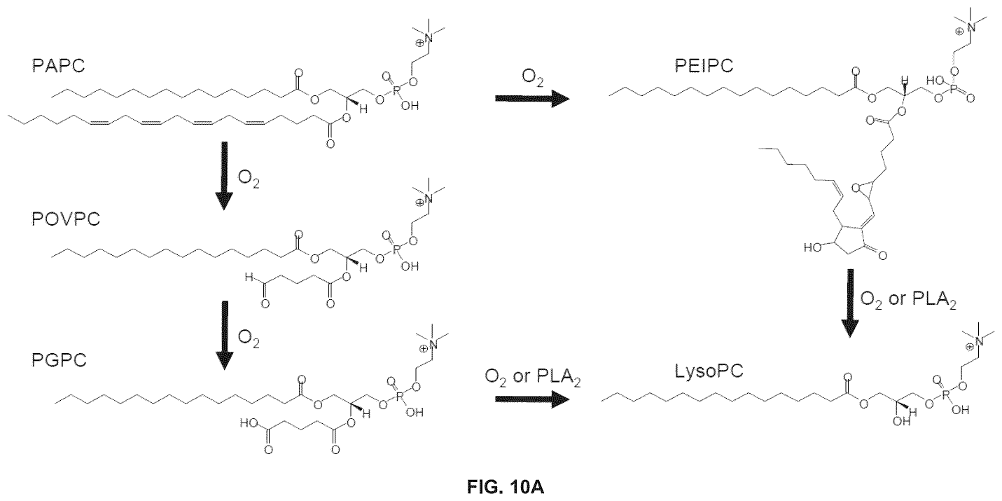

To interrogate the upstream regulation of caspase-4, we focused on interferon signaling, which is involved in activation of the caspase-11 driven non-canonical inflammasome^{11,17}. Alu RNA did not induce RPE degeneration or caspase-11 activation in Ifnar1^{-/-} mice or Ifnar1^{-/-} mouse RPE cells (FIGS. 3A and 3B), which are deficient in the type I interferon-α/β receptor (IFNAR). Recombinant interferon-β increased caspase-4 abundance in human RPE cells (FIG. 3C). Alu RNA induced secretion of interferon-β (FIG. 3D) and phosphorylation of IRF3 (Supplementary FIG. 9A), a transcription factor that induces production of interferon-β. Alu RNA also induced phosphorylation of STAT2 (FIG. 3E and Supplementary FIGS. 9A and 9B), a signaling molecule activated by type-I interferons downstream of IFNAR. Alu RNA also did not induce RPE degeneration in Irf3^{-/-} or Stat2^{-/-} mice (FIG. 3F and Supplementary FIGS. 9C and 9D), and its induction of caspase-11 activation was reduced in Stat2^{-/-} mouse RPE cells (Supplementary FIG. 9E). Alu RNA-induced RPE degeneration was blocked by administration of an IFN-β neutralizing antibody (FIG. 3G), demonstrating that IFN-β is critical in Alu toxicity.

Human eyes with geographic atrophy displayed pronounced IFN-β expression in the RPE compared to unaffected aged human eyes (FIGS. 3H and 3I). Collectively these findings suggest that Alu RNA-induced RPE degeneration is dependent on type I interferon signaling-regulated non-canonical NLRP3 inflammasome, and that this signaling provides the conditions required for caspase-11 induction and activation.

cGAS-Driven IFN Signaling Licenses Non-Canonical NLRP3 Inflammasome

We sought to identify the upstream activator of IRF3-driven interferon signaling induced by Alu RNA. Alu RNA-induced RPE degeneration is independent of several IRF3-activating signaling molecules including various RNA sensors: TLR3, TLR4, TLR9, RIG-I, MDA5, MAVS, and TRIF². Cyclic GMP-AMP synthase (cGAS; encoded by Mb21d1), has emerged as an innate immune sensor that can activate type I interferon signaling⁴¹⁻⁴³. Additionally, a role for cGAS in setting the type I IFN threshold to RNA virus infection has also been reported^{44,45}.

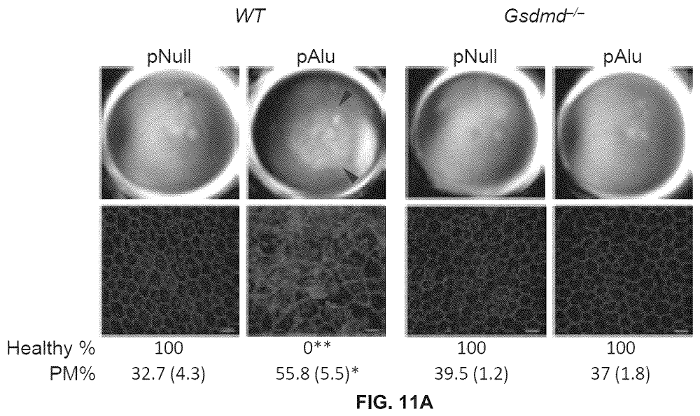

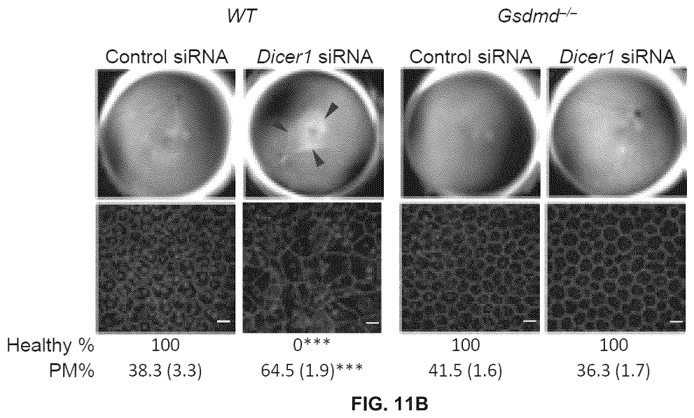

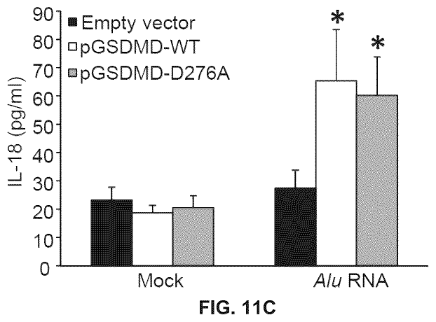

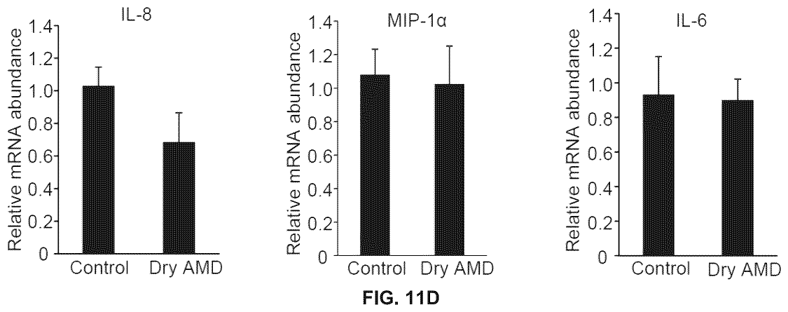

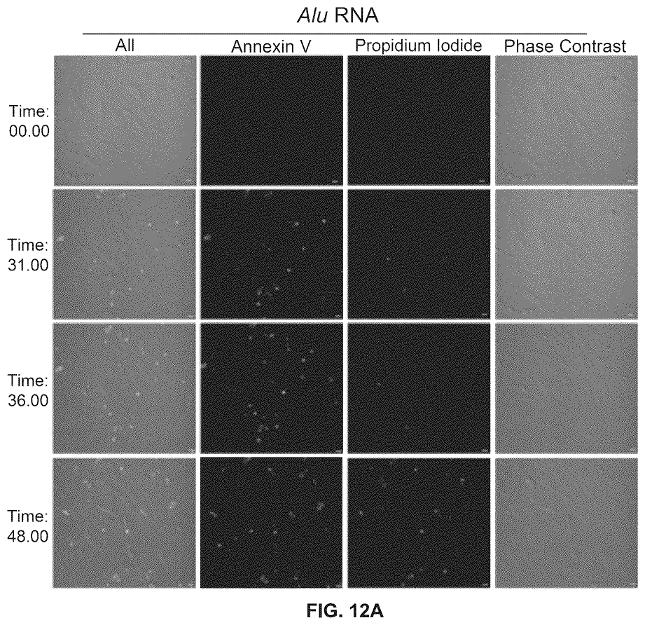

Alu RNA upregulated cGAS mRNA and protein in human RPE cells (Supplementary FIGS. 10A and 10B). In contrast to WT mouse RPE cells, Alu RNA did not induce interferon-β (FIG. 4A), activate caspase-1 (FIG. 4B and Supplementary FIG. 10C) or caspase-11 (FIG. 4C), or induce IL-18 secretion (FIG. 4D) in Mb21d1^{-/-} mouse RPE cells. Inflammasome activation by MSU crystals remained unaffected in Mb21d1^{-/-} mouse RPE cells (Supplementary FIG. 10D). DICER1 knockdown in human RPE cells, which leads to interferon-β induction, STAT2 phosphorylation, and activation of caspase-4 and caspase-1, were all inhibited by knockdown of cGAS (FIGS. 4E and 4F and Supplementary FIGS. 10E and 10F). Corroborating these data, Alu RNA did not induce RPE degeneration in Mb21d1^{-/-} mice (FIG. 4G and Supplementary FIGS. 10G and 10H). Additionally, reconstitution with ectopic mouse cGAS restored IFN-β induction in Mb21d1^{-/-} mouse RPE cells and RPE degeneration in Mb21d1^{-/-} mice (FIG. 4H, Supplementary FIG. 101, and Supplementary FIG. 11A). The resistance of Mb21d1^{-/-} mice to Alu RNA-induced RPE degeneration was overcome by recombinant IFN-β administration or IFN-β expression via subretinal plasmid transfection, suggesting that the loss of susceptibility to Alu RNA in these mice is indeed due to lack of IFN signaling (FIG. 4I).

We observed increased abundance of cGAS protein in the RPE of human geographic atrophy eyes compared to unaffected aged eyes (FIG. 5A). cGAS-driven interferon signaling can be transduced by the adaptor protein STING (encoded by Tmem173)^{42,43,46}. Alu RNA did not induce IRF3 phosphorylation (Supplementary FIGS. 11B and 11C) or activation of caspase-1 (FIG. 5B) and caspase-11 (FIG. 5C) in Tmem173^{-/-} mouse RPE cells, and did not induce RPE degeneration in Tmem173^{-/-} mice (FIG. 5D), pointing to the involvement of the cGAS-STING signaling axis in this system. The resistance of Tmem173^{-/-} mice to Alu RNA-induced RPE degeneration was overcome by recombinant IFN-β administration or IFN-β expression via subretinal plasmid transfection, suggesting that the loss of susceptibility to Alu RNA in these mice is indeed due to lack of IFN signaling (FIG. 5E).

Alu-Driven cGAS Activation is Triggered by Engagement with mtDNA

CGAS is activated by cytosolic DNA but not by poly(I:C), a synthetic double stranded RNA analog⁴³. Consistent with the notion that cGAS does not recognize RNA directly, Alu RNA did not bind cGAS in an RNA immunoprecipitation assay. Previous studies have implicated mitochondrial dysfunction in macular degeneration including mitochondrial DNA (mtDNA) damage, reactive oxygen species (ROS) production, and downregulation of proteins involved in mitochondrial energy production and trafficking^{2,47,48}. Cytosolic escape of mitochondrial components such as DNA and formyl peptides activates innate immune pathways including cGAS (refs. 49,50).

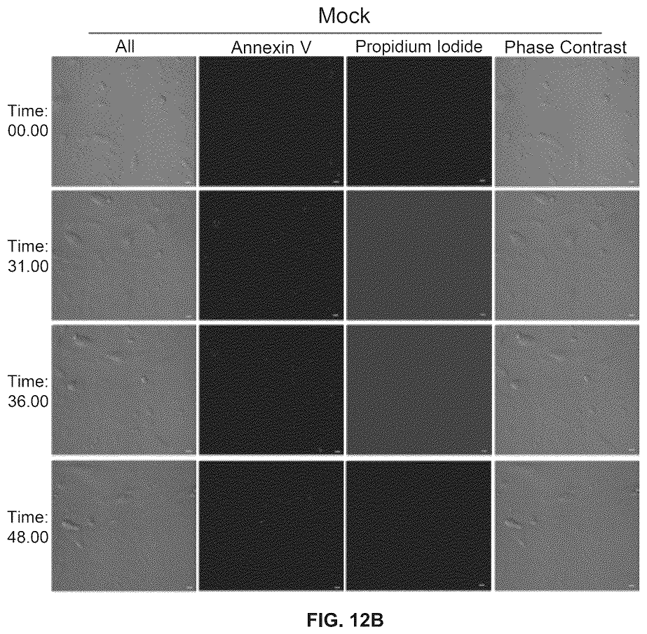

Both Alu RNA stimulation and DICER1 knockdown in human RPE cells resulted in increased cytosolic abundance of mtDNA (FIG. 6A and Supplementary FIGS. 12A and 12B). To examine whether Alu RNA triggers engagement of mtDNA by cGAS, we performed a DNA-protein interaction pull down assay in HA-tagged cGAS reconstituted Mb21d1^{-/-} immortalized mouse embryonic fibroblasts⁴⁹. As these cells express HA-cGAS from a genomically integrated DNA sequence, they would be expected to mimic endogenous cGAS expression. We observed enrichment of mtDNA in cGAS immunoprecipitate of Alu RNA-stimulated but not mock- or poly(I:C)-stimulated cells (FIG. 6B and Supplementary FIG. 12C), suggesting that mtDNA in the cytosol engages cGAS. As a positive control, transfected plasmid DNA in this assay was also enriched in the cGAS immunoprecipitate (Supplementary FIG. 12D). Additionally, subretinal delivery of mtDNA induced RPE degeneration in WT but not in Mb21d1^{-/-} mice (Supplementary FIG. 12E). Similarly, in cell culture studies, mtDNA-induced Ifnb mRNA was reduced in Mb21d1^{-/-} compared to WT mouse RPE cells (Supplementary FIG. 12F).

Mitochondrial Permeability Transition Pore is Required for Alu-Driven mtDNA Release

During conditions of cellular stress, opening of the mitochondrial permeability transition pore (mPTP) leads to mitochondrial swelling, rupture, and release of mitochondrial contents into the cytosol^{51,52}. In cells lacking mitochondrial peptidyl-prolyl cis-trans isomerase F (PPIF, also known as cyclophilin D), a key enzyme involved in mPTP, mitochondria are resistant to swelling and permeability transition^{53,54}. We assessed whether Alu RNA induced mPTP opening using the JC-1 and cobalt-calcein assays⁵⁵. Alu RNA induced a reduction of mitochondrial membrane potential (ΔΨm), as determined by the potential-sensitive fluorochrome JC-1, and quenching of the calcein signal in wild-type but not Ppif^{-/-} mouse RPE cells (Supplementary FIGS. 12G and 12H). In addition, cyclosporine A, which inhibits mPTP opening via binding to PPIF, blocked Alu RNA-induced mPTP opening in wild-type cells, but did not alter ΔΨm or calcein intensity in Ppif^{-/-} cells (Supplementary FIGS. 12G and 12H). Collectively, these findings suggest that Alu RNA induces Ppif^{-/-} dependent mPTP opening in RPE cells.

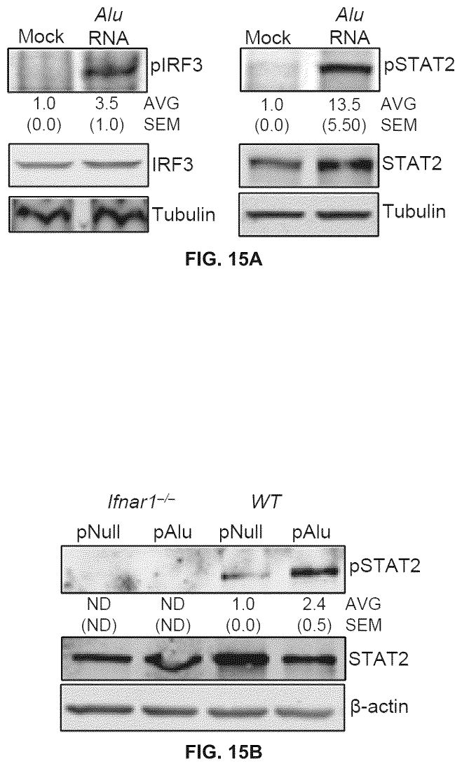

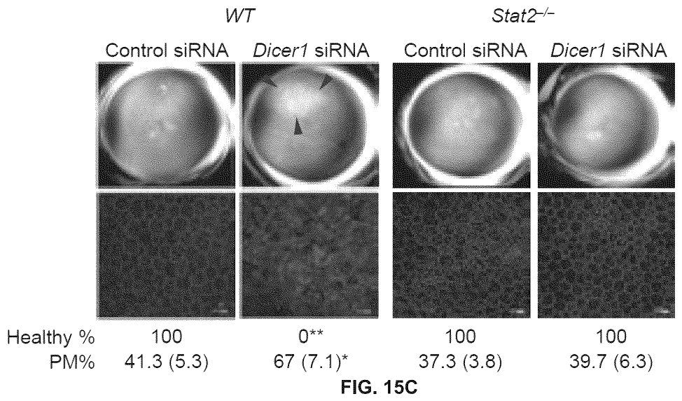

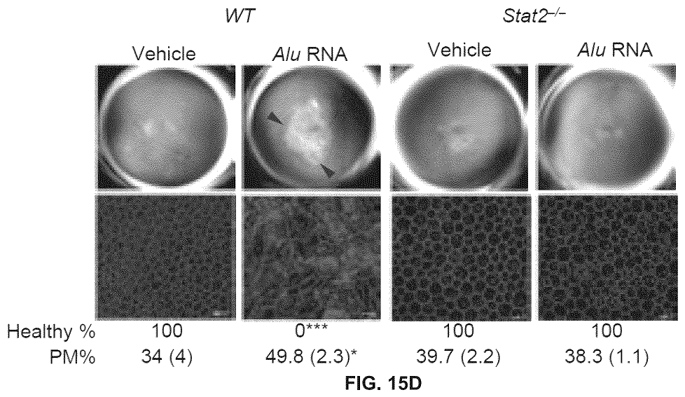

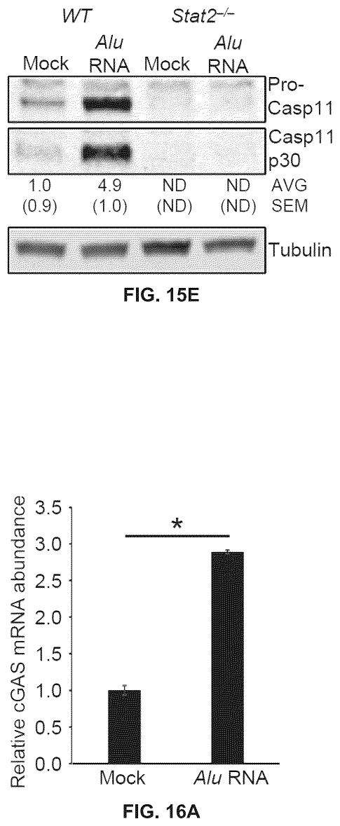

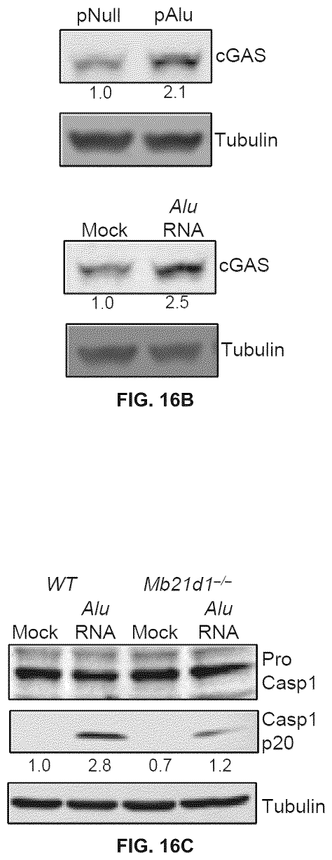

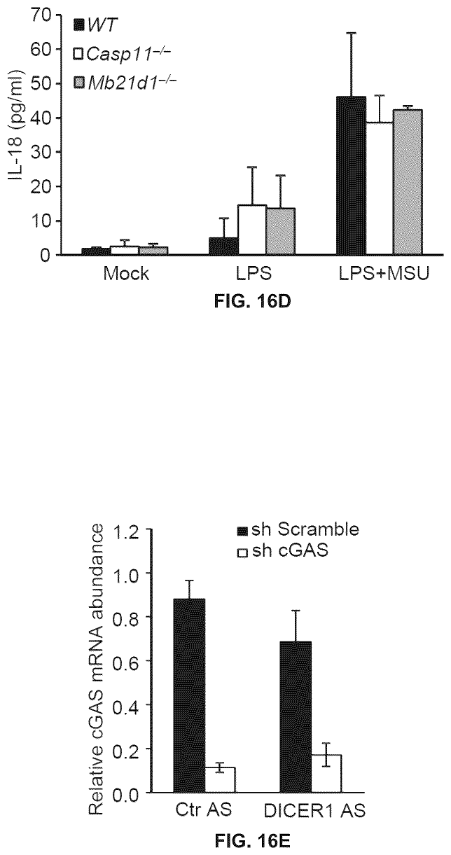

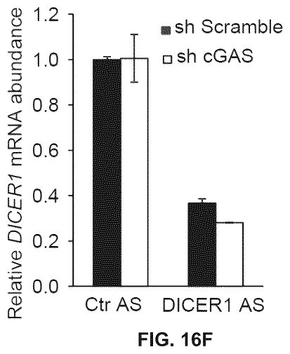

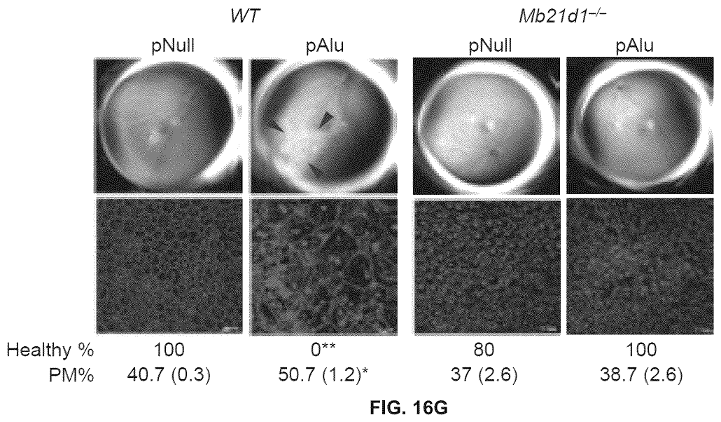

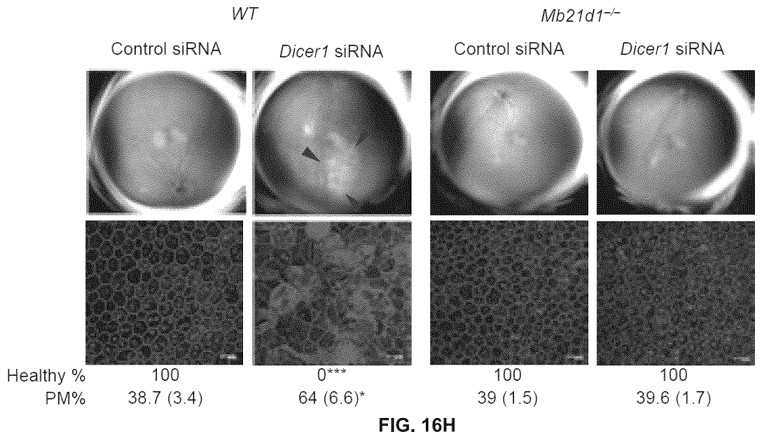

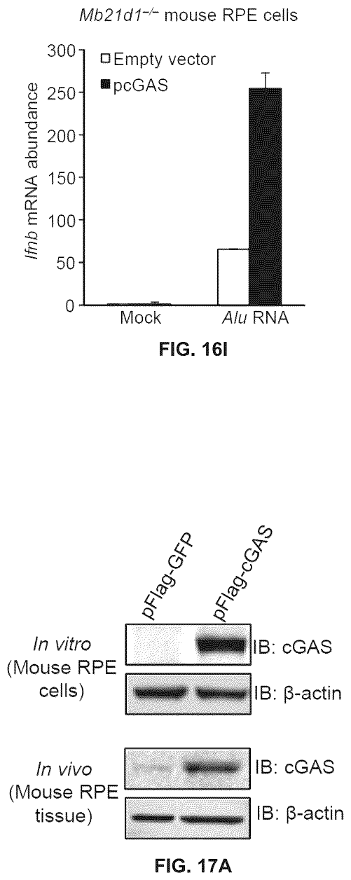

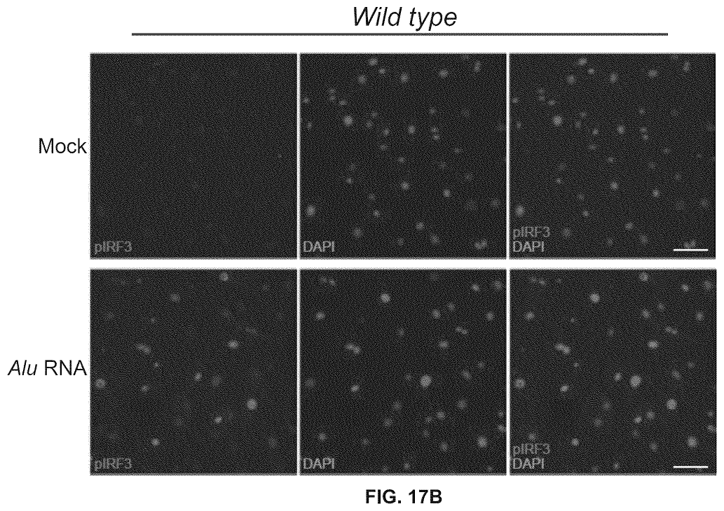

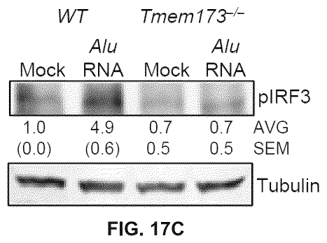

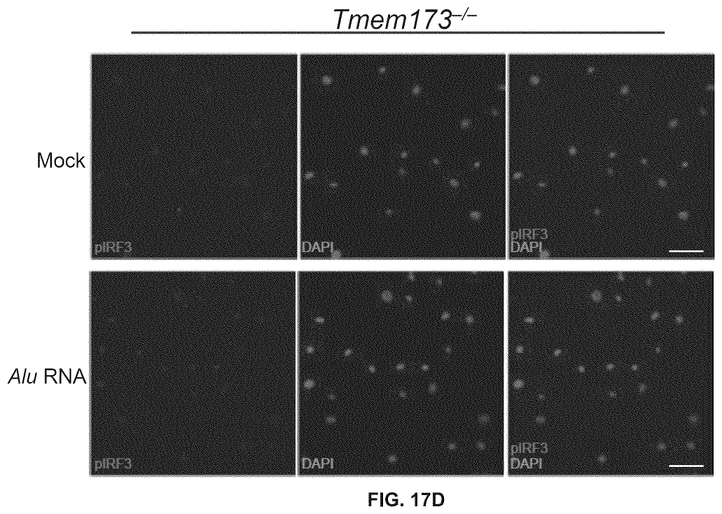

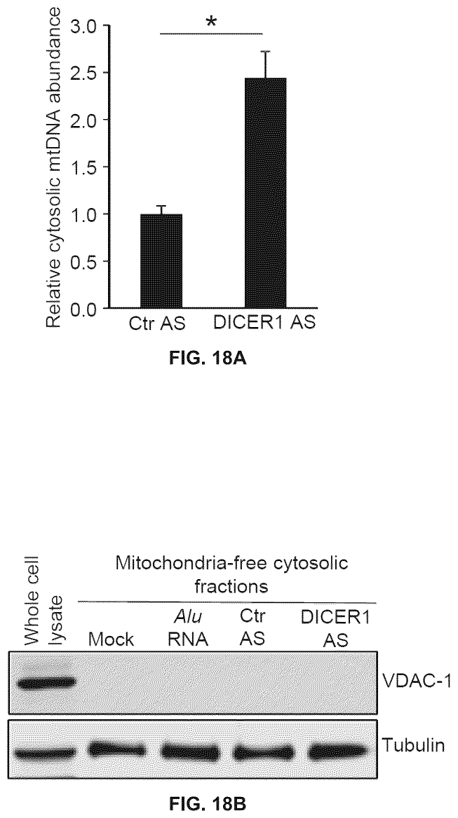

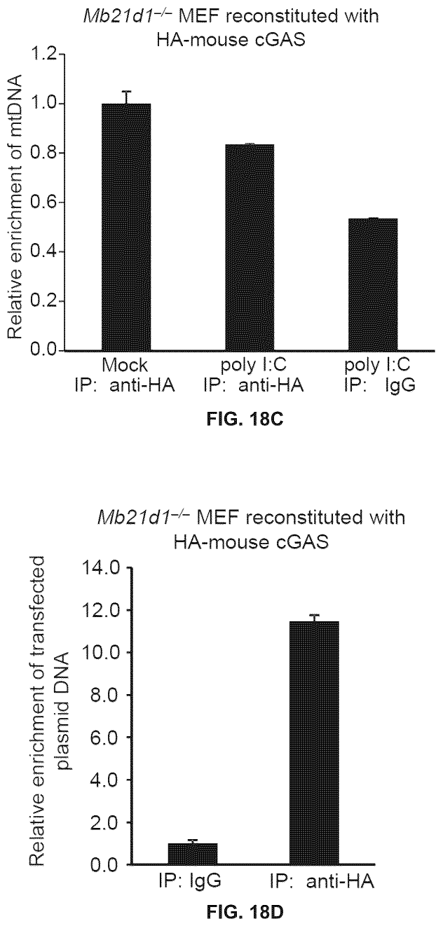

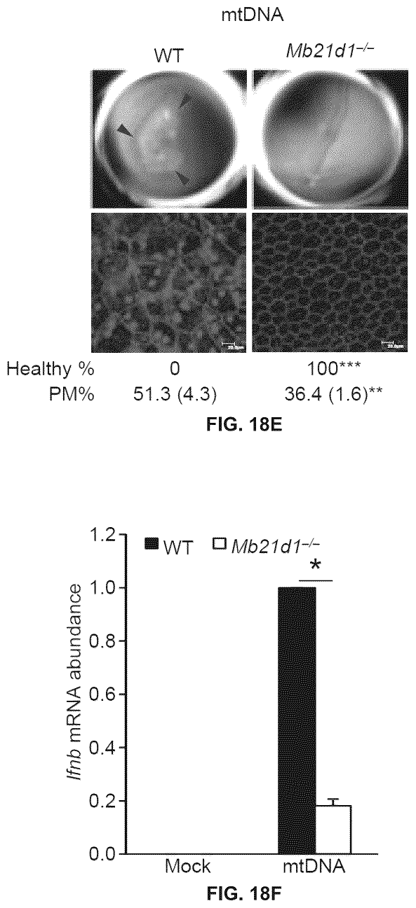

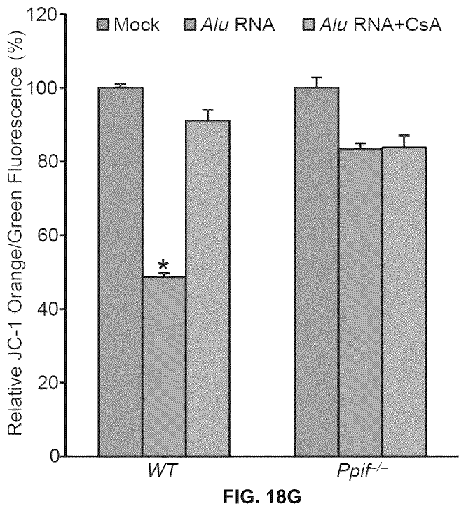

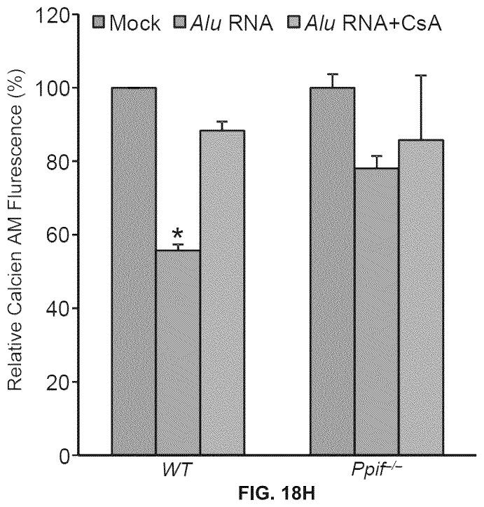

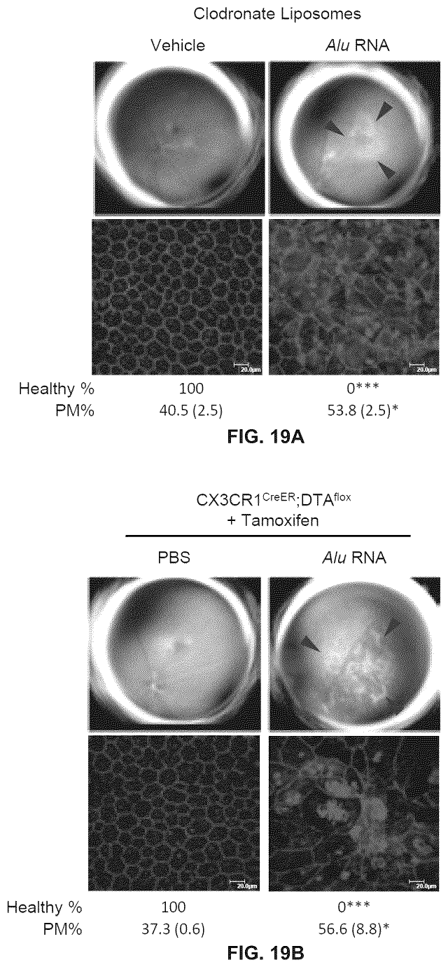

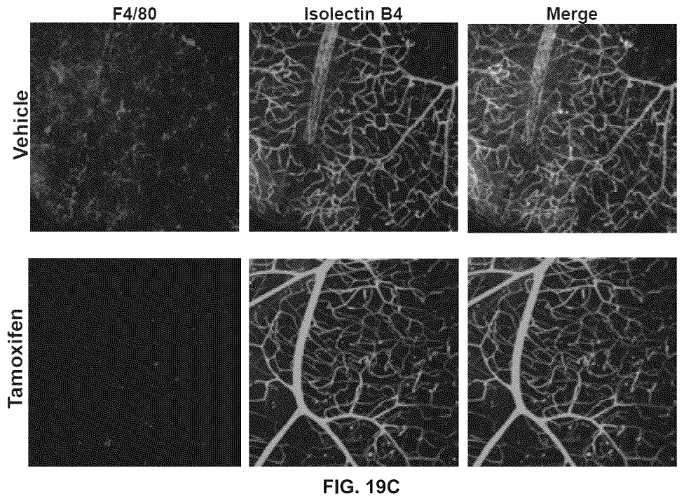

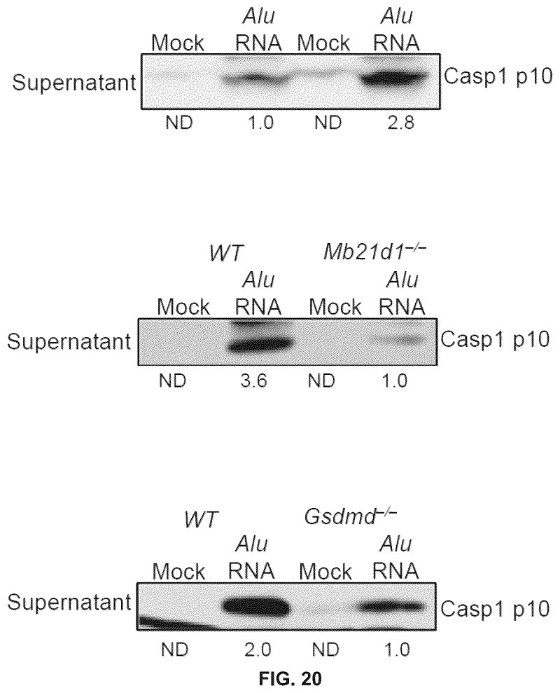

Alu RNA triggered mtDNA release into the cytosol in WT but not Ppif^{-/-} mouse RPE cells (FIG. 6C). Ppif^{-/-} mice were protected against Alu RNA-induced RPE degeneration (FIG. 6D), confirming the in vivo importance of mPTP in Alu toxicity. Alu RNA-induced activation of caspase-1 and caspase-11 were reduced in Ppif^{-/-} mouse RPE cells (FIGS. 6E and 6F). In human RPE cells lacking mitochondrial DNA (Rho⁰ ARPE19)⁵⁶, Alu RNA no longer activated caspase-4 (FIG. 6G) or induced secretion of IL-18 (FIG. 6H) or IFN-β (FIG. 6I). Furthermore, the resistance of Ppif^{-/-} mice to Alu RNA-induced RPE degeneration was overcome by recombinant IFN-β administration or IFN-β expression via subretinal plasmid transfection, suggesting that the loss of susceptibility to Alu RNA in these mice is indeed due to lack of IFN signaling (FIG. 6J). Collectively these data support a model wherein mPTP-driven mitochondrial permeability mediates cytosolic release of mtDNA, which in turn promotes non-canonical NLRP3 inflammasome via engaging the cytosolic DNA sensor cGAS-driven IFN signaling (Supplementary FIG. 15).

Alu Driven RPE Toxicity does not Require Macrophages or Microglia

We focused on the RPE as the cellular locus of inflammasome activation because we previously demonstrated the localization of DICER1 deficiency, Alu RNA accumulation, and increased abundance of NLRP3, PYCARD, cleaved caspase-1, and phosphorylated IRAK1/4 to the RPE layer of human eyes with geographic atrophy^{2,3}. Our current observations of elevated cGAS, gasdermin D, cleaved caspase-4, and IFN-β in the RPE in diseased eyes further buttress this cell layer as the locus of molecular perturbations in the non-canonical inflammasome pathway in geographic atrophy.

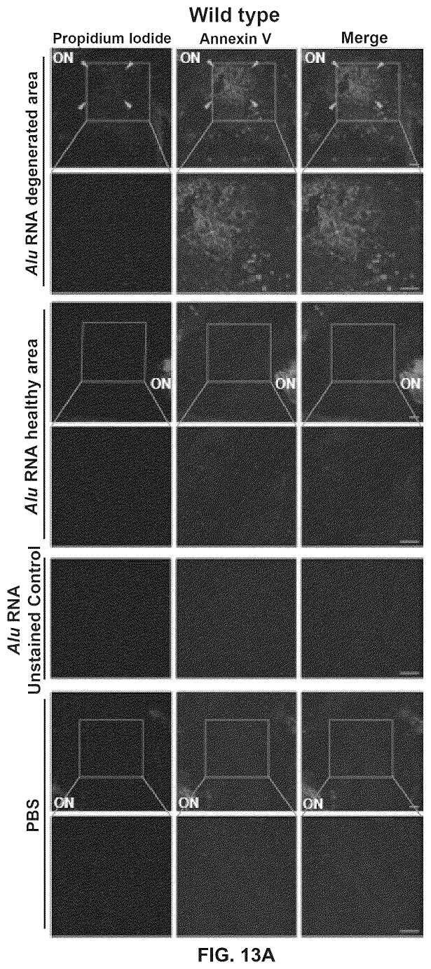

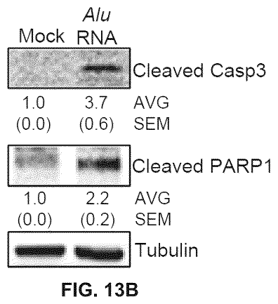

However, there are recent reports that macrophages and microglia can be observed in the vicinity of pathology in human eyes with geographic atrophy⁵⁷⁻⁵⁹. Previously we demonstrated that RPE cell-specific ablation of Myd88, the adaptor critical for IL-18-induced RPE cell death in this system, was sufficient to prevent Alu RNA-induced RPE degeneration in mice². We also demonstrated using mouse chimeras that ablation of Myd88 in circulating bone marrow derived cells did not prevent Alu RNA-induced RPE degeneration². Nevertheless, given that these professional immune cells are capable of inflammasome signaling, we studied their involvement more directly. We depleted macrophages using clodronate liposomes⁶⁰ and depleted microglia by administering tamoxifen to Cx3cr1^{CreER} ROSA-DTA mice⁶¹. Alu RNA induced RPE degeneration in mice despite depleting macrophages or microglia, providing direct evidence that these two cell populations are dispensable for RPE toxicity in this system (Supplementary FIG. 13).

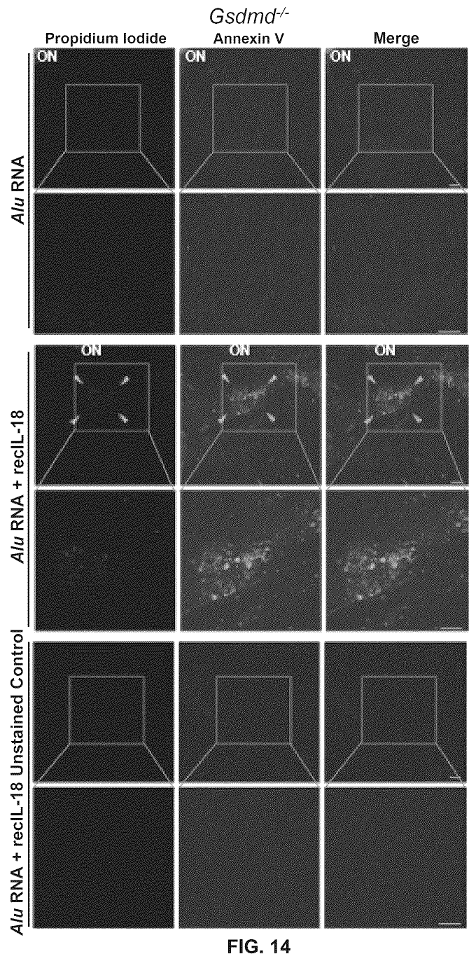

Although these two cell types are apparently not required by Alu RNA to elicit RPE degeneration in mice, it is possible that they might subtly influence disease pathology in this system. Indeed we found that Alu RNA activates the non-canonical inflammasome in mouse BMDMs (Supplementary FIG. 14). Alike in RPE cells, Alu RNA-induced caspase-1 activation was reduced in Casp11^{-/-}, Mb21d1^{-/-}, and Gsdmd^{-/-} BMDMs (Supplementary FIG. 14). Collectively these findings suggest that cGAS-driven licensing of the non-canonical NLRP3 inflammasome by Alu RNA is not restricted to RPE cells.

### DISCUSSION

Our data identify an unexpected role for the DNA sensor cGAS, the non-canonical caspase-4 inflammasome, and gasdermin D in mediating Alu RNA-induced RPE cell death in various mouse models and human cell culture systems. Coupled with the increased abundance of cGAS, interferon-β, caspase-4, and gasdermin D in the RPE of human geographic atrophy eyes, our findings point to their involvement in the pathogenesis of this form of age-related macular degeneration and provide new targets for treatment and prevention.

cGAS was originally recognized as sensor of exogenous and endogenous cytosolic DNA that mediates IRF3-driven interferon signaling, and previous studies demonstrated that the enzymatic activity of cGAS could not be activated by an RNA stimulus⁴³. Nonetheless, cGAS has been reported to be critical for the antiviral response to multiple RNA viruses^{44,45}; although the mechanistic underpinnings of this effect are not fully understood, our work defines a novel pathway by which endogenous RNAs can activate cGAS in a model of a prevalent human disease.

Our findings also raise the possibility that cGAS-driven antiviral immunity involves Alu RNA, which can be stimulated by viral infections⁶²⁻⁶⁶. Mitochondria have been increasingly implicated as gatekeepers of cell fate with decisive roles in diverse cellular responses including apoptosis, autophagy, and innate immunity^{67,68}. Mitochondria can facilitate the innate immune response to infection and injury via release of mitochondrial components as DAMPs that can be recognized by the cell's innate immune components. Of note, mtDNA can activate multiple arms of innate immunity including the NLRP3 inflammasome, TLR9, and cGAS/STING-driven IFN signaling^{50,69}. mtDNA can activate the NLRP3 inflammasome by directly interacting with NLRP3 (ref. 70) or amplifying the response to an initial trigger such as ATP or ROS⁷¹.

In addition, mtDNA can activate TLR9 on neutrophils triggering systemic lung and liver inflammation⁷²⁻⁷⁴. Besides engaging TLR9 and NLRP3 signaling, mtDNA has also recently been reported in the activation of cGAS signaling by cytosolic escape of mtDNA as a consequence of mitochondrial stress⁴⁹. Interestingly we previously demonstrated that TLR9 signaling is dispensable for Alu RNA-induced RPE degeneration², and that NLRP3 inflammasome priming is unaffected in mouse RPE cells lacking TLR9 (ref. 20). Therefore our findings implicating activation of both NLRP3 and cGAS signaling pathways under the conditions of Alu RNA-driven cytosolic mtDNA release highlight the significance of the mitochondria as a signaling platform that integrates various cellular stress cues into innate immune signaling in autoimmune and chronic inflammatory diseases.

In addition to its role in responding to infections, cGAS has been implicated in mouse models of autoimmune diseases and mouse tumor models. Our findings that cGAS is elevated in the RPE of human eyes with AMD and is critical for Alu RNA-induced RPE degeneration in mice and in human cells expands the functional repertoire of this innate immune sensor to chronic degenerative diseases.

Caspase-4/11-mediated activation of the non-canonical NLRP3 inflammasome has been implicated in gram-negative bacterial infection, sepsis, and antimicrobial defense at the mucosal surface^{11-17,28}. To our knowledge, our report is the first example of caspase-4-driven non-canonical inflammasome activation in a non-infectious human disease. Also it bears investigating whether caspase-4 and cGAS are involved in other conditions such as systemic lupus erythematosus and diabetes mellitus, wherein Alu RNA accumulation has been observed^{75,76}. Activation of caspase-4 has been observed in conditions of endoplasmic reticulum (ER) stress⁷⁷; interestingly, several human diseases including Alzheimer's disease, and obesity driven-type 2 diabetes, which are also driven by hyperactive inflammasome, are associated with ER stress^{78,79}. It would be revealing to explore whether DICER1 deficit or Alu RNA-induced mitochondrial dysfunction and cGAS- and caspase-4 dependent-inflammasome activation are linked to ER stress.

Our studies also reveal that gasdermin D lies mechanistically downstream of caspase-11 activation and is required for Alu toxicity. Of note, the role of gasdermin D in this system appears not to be induction of pyroptosis, as it is in response to exogenous triggers such as intracellular LPS. Instead, gasdermin D supports Alu RNA-induced RPE cell apoptosis by promoting IL-18 secretion without being cleaved into its p30 fragment, which is required for its pyroptotic effect. To our knowledge, this is the first report of gasdermin D involvement in a non-infectious human disease. Interestingly, Fas/FasL are thought to play a critical role in limiting inflammation in immune-privileged sites such as the eye⁸⁰. Therefore it is conceivable that additional mechanisms have evolved to limit inflammasome-driven gasdermin D-mediated pore formation and pyroptotic cell death that would otherwise exacerbate inflammation via release of DAMPs leading to recruitment of inflammatory cells into the eye. Hence inflammasomes in the eye might be geared towards engaging IL-18-promoted, Fas/FasL-driven pro-apoptotic cell death. Additional studies are required to dissect the mechanisms that disengage the pore-forming function of gasdermin D from the inflammasome-activating function, i.e., caspase-1 activation and IL-18 secretion. Furthermore pyroptosis induction via gasdermin D upon non-canonical inflammasome activation could be dictated by the activating trigger (e.g. exogenous versus host) or the cell type involved. For instance, the only other endogenous molecule known to activate caspase-11, oxPAPC, also did not induce pyroptosis, yet triggered IL-1β release from dendritic cells¹⁸. Interestingly, Alu RNA induces oxPAPC synthesis, raising the possibility that Alu RNA might recruit other DAMPs to induce RPE toxicity.

The molecular mechanism by which caspase-8 influences inflammasome activation is elusive. Caspase-8 has been reported to both prime the NLRP3 inflammasome as well as trigger cleavage of pro-IL-18 and pro-IL-1β, either with or without caspase-1 (ref 81). In our model, caspase-8 mediated Alu toxicity was mediated by IL-18-driven Fas/FasL²¹ (see also FIG. 21 and Supplementary FIG. 15). In another study, both canonical and non-canonical inflammasome were regulated by FADD and caspase-8 signaling at the level of both inflammasome priming and activation⁸². Therefore a model is conceivable wherein an initial round of caspase-1 activation and IL-18 secretion set in motion a cascade of events that can be further modulated to achieve signal amplification in a feed-forward manner by recruiting additional molecules such as FasL and caspase-8.

The mechanisms underlying regulation of the NLRP3 inflammasome by caspase-11/4 have been elusive. Previously we demonstrated that Alu RNA-induced RPE degeneration and NLRP3 inflammasome activation depended on NF-κB and P2X7 (refs. 19, 20). Surprisingly in our studies Alu RNA-induced caspase-11 activation was subdued in P2rx7^{-/-} mouse RPE cells, suggesting that P2X7 is required for both caspase-1 and caspase-11 activation. A role for P2X7 also has been reported in a mouse model of caspase-11-dependent endotoxic shock⁸³. Taken together, our observations suggest that Alu RNA-driven NLRP3 inflammasome activation engages both caspase-11/4 and P2X7. We also observed that Alu RNA induces oxPAPC synthesis, suggesting that Alu RNA might recruit other DAMPs to activate the non-canonical inflammasome during RPE cell toxicity. Earlier reports have implicated oxidized phospholipids in the pathophysiology of age-related macular degeneration⁸⁴⁻⁸⁶; future studies should unravel the molecular details of this pathway.

In recent years, numerous groups employing a variety of cell culture systems, animal models, and human donor eyes have reported an important role for the inflammasome in AMD^{2,6-10,87-89} Collectively these studies suggest that NLRP3 pathway is an important responder to a panoply of AMD-related molecular stressors and toxins in RPE cells. Thus there is great interest in inflammasome inhibition as a therapeutic for AMD¹⁹. Our identification of cGAS, interferon-β, caspase-4, and gasdermin D as critical mediators in inflammasome-driven RPE degeneration expands the array of therapeutic targets for AMD.

Although there is consensus that NLRP3 inflammasome activation is detrimental to RPE cell health and survival, akin to that of other cell types^{81,90}, there is controversy about the role of this pathway in neovascular AMD. It has been reported that IL-18, a cytokine produced by NLRP3 inflammasome activation, inhibits angiogenesis and that IL-18 neutralization augments angiogenesis in a laser injury model of choroidal neovascularization⁸⁸⁻⁸⁹. However, an international consortium did not replicate this anti-angiogenic effect of IL-18 and also demonstrated that the promotion of angiogenesis by an IL-18 antibody was due to an excipient in its preparation⁹¹. These conflicting data in neovascular AMD models do not, however, impact the conclusion that inflammasome activation promotes RPE degeneration, which provides a mechanistic rationale for testing inflammasome inhibition in geographic atrophy⁹¹.

Our finding that DICER1, through its cleavage of Alu RNA, can prevent activation of the non-canonical inflammasome adds to the functionality of this multifaceted protein that has microRNA biogenesis, anti-apoptotic, and tumor-related functions. Although it is unknown why DICER1 levels might be reduced in AMD³, DICER1 protein levels are suppressed by hypoxia, type I interferons, dsRNAs, and reactive oxygen species^{92,93}, all of which are thought to contribute to AMD pathogenesis^{94,95}.

In summary, our studies have uncovered a novel effect of cGAS signaling in response to endogenous retroelement transcripts, which involves the unexpected collaboration between mitochondrial dysfunction, cGAS-driven interferon signaling, gasdermin D, and NLRP3 inflammasome activation (See FIG. 21 and Supplementary FIG. 15). Targeting this pathway or various components of the pathway presents potentially a new therapeutic approach to preserve RPE health in age-related macular degeneration and a host of other inflammasome-driven diseases.

The disclosures of each and every patent, patent application, and publication cited herein are hereby incorporated by reference herein in their entirety.

Headings are included herein for reference and to aid in locating certain sections. These headings are not intended to limit the scope of the concepts described therein under, and these concepts may have applicability in other sections throughout the entire specification.

While this invention has been disclosed with reference to specific embodiments, it is apparent that other embodiments and variations of this invention may be devised by others skilled in the art without departing from the true spirit and scope of the invention.

### BIBLIOGRAPHY FOR RESULTS

1. Ambati, J., Ambati, B. K., Yoo, S. H., Ianchulev, S. & Adamis, A. P. Age-related macular degeneration: etiology, pathogenesis, and therapeutic strategies. Surv Ophthalmol 48, 257-293 (2003).
2. Tarallo, V., et al. DICER1 loss and Alu RNA induce age-related macular degeneration via the NLRP3 inflammasome and MyD88. Cell 149, 847-859 (2012).
3. Kaneko, H., et al. DICER1 deficit induces Alu RNA toxicity in age-related macular degeneration. Nature 471, 325-330 (2011).
4. Wong, W. L., et al. Global prevalence of age-related macular degeneration and disease burden projection for 2020 and 2040: a systematic review and meta-analysis. Lancet Glob Health 2, e106-116 (2014).
5. Ambati, J., Atkinson, J. P. & Gelfand, B. D. Immunology of age-related macular degeneration. Nat Rev Immunol 13, 438-451 (2013).
6. Anderson, O. A., Finkelstein, A. & Shima, D. T. A2E induces IL-1ss production in retinal pigment epithelial cells via the NLRP3 inflammasome. PLoS One 8, e67263 (2013).
7. Brandstetter, C., Mohr, L. K., Latz, E., Holz, F. G. & Krohne, T. U. Light induces NLRP3 inflammasome activation in retinal pigment epithelial cells via lipofuscin-mediated photooxidative damage. J Mol Med (Berl) 93, 905-916 (2015).
8. Kauppinen, A., et al. Oxidative stress activates NLRP3 inflammasomes in ARPE-19 cells—implications for age-related macular degeneration (AMD). Immunol Lett 147, 29-33 (2012).
9. Tseng, W. A., et al. NLRP3 Inflammasome Activation in Retinal Pigment Epithelial Cells by Lysosomal Destabilization: Implications for Age-Related Macular Degeneration. Invest Ophthalmol Vis Sci 54, 110-120 (2013).
10. Gelfand, B. D., et al. Iron Toxicity in the Retina Requires Alu RNA and the NLRP3 Inflammasome. Cell Rep 11, 1686-1693 (2015).
11. Broz, P., et al. Caspase-11 increases susceptibility to Salmonella infection in the absence of caspase-1. Nature 490, 288-291 (2012).
12. Casson, C. N., et al. Human caspase-4 mediates noncanonical inflammasome activation against gram-negative bacterial pathogens. Proc Natl Acad Sci USA 112, 6688-6693 (2015).
13. Hagar, J. A., Powell, D. A., Aachoui, Y., Ernst, R. K. & Miao, E. A. Cytoplasmic LPS activates caspase-11: implications in TLR4-independent endotoxic shock. Science 341, 1250-1253 (2013).
14. Kayagaki, N., et al. Non-canonical inflammasome activation targets caspase-11. Nature 479, 117-121 (2011).
15. Kayagaki, N., et al. Noncanonical inflammasome activation by intracellular LPS independent of TLR4. Science 341, 1246-1249 (2013).
16. Knodler, L. A., et al. Noncanonical inflammasome activation of caspase-4/caspase-11 mediates epithelial defenses against enteric bacterial pathogens. Cell Host Microbe 16, 249-256 (2014).
17. Rathinam, V. A., et al. TRIF licenses caspase-11-dependent NLRP3 inflammasome activation by gram-negative bacteria. Cell 150, 606-619 (2012).
18. Zanoni, I., et al. An endogenous caspase-11 ligand elicits interleukin-1 release from living dendritic cells. Science 352, 1232-1236 (2016).
19. Fowler, B. J., et al. Nucleoside reverse transcriptase inhibitors possess intrinsic anti-inflammatory activity. Science 346, 1000-1003 (2014).
20. Kerur, N., et al. TLR-independent and P2X7-dependent signaling mediate Alu RNA-induced NLRP3 inflammasome activation in geographic atrophy. Invest Ophthalmol Vis Sci 54, 7395-7401 (2013).
21. Kim, Y., et al. DICER1/Alu RNA dysmetabolism induces Caspase-8-mediated cell death in age-related macular degeneration. Proc Natl Acad Sci USA 111, 16082-16087 (2014).
22. Huang, J., et al. Comparison of Noncontact Specular and Confocal Microscopy for Evaluation of Corneal Endothelium. Eye Contact Lens, doi: 10.1097/ICL.0000000000000362 (2017).
23. Ach, T., et al. Lipofuscin redistribution and loss accompanied by cytoskeletal stress in retinal pigment epithelium of eyes with age-related macular degeneration. Invest Ophthalmol Vis Sci 56, 3242-3252 (2015).
24. Dridi, S., et al. ERK1/2 activation is a therapeutic target in age-related macular degeneration. Proc Natl Acad Sci USA 109, 13781-13786 (2012).
25. Grossniklaus, H. E., Nickerson, J. M., Edelhauser, H. F., Bergman, L. A. & Berglin, L. Anatomic alterations in aging and age-related diseases of the eye. Invest Ophthalmol Vis Sci 54, ORSF23-27 (2013).
26. Kleinman, M. E., et al. Sequence- and target-independent angiogenesis suppression by siRNA via TLR3. Nature 452, 591-597 (2008).
27. Kleinman, M. E., et al. Short-interfering RNAs induce retinal degeneration via TLR3 and IRF3. Mol Ther 20, 101-108 (2012).
28. Kajiwara, Y., et al. A critical role for human caspase-4 in endotoxin sensitivity. J Immunol 193, 335-343 (2014).
29. Zhao, L., et al. Microglial phagocytosis of living photoreceptors contributes to inherited retinal degeneration. EMBO Mol Med 7, 1179-1197 (2015).
30. Shi, J., et al. Cleavage of GSDMD by inflammatory caspases determines pyroptotic cell death. Nature 526, 660-665 (2015).
31. Kayagaki, N., et al. Caspase-11 cleaves gasdermin D for non-canonical inflammasome signalling. Nature 526, 666-671 (2015).
32. Ding, J., et al. Pore-forming activity and structural autoinhibition of the gasdermin family. Nature 535, 111-116 (2016).
33. Liu, X., et al. Inflammasome-activated gasdermin D causes pyroptosis by forming membrane pores. Nature 535, 153-158 (2016).
34. Sborgi, L., et al. GSDMD membrane pore formation constitutes the mechanism of pyroptotic cell death. EMBO J 35, 1766-1778 (2016).
35. Aglietti, R. A., et al. GsdmD p30 elicited by caspase-11 during pyroptosis forms pores in membranes. Proc Natl Acad Sci USA 113, 7858-7863 (2016).
36. Dunaief, J. L., Dentchev, T., Ying, G. S. & Milam, A. H. The role of apoptosis in age-related macular degeneration. Arch Ophthalmol 120, 1435-1442 (2002).
37. van Genderen, H., et al. In vitro measurement of cell death with the annexin A5 affinity assay. Nat Protoc 1, 363-367 (2006).
38. Wlodkowic, D., Telford, W., Skommer, J. & Darzynkiewicz, Z. Apoptosis and beyond: cytometry in studies of programmed cell death. Methods Cell Biol 103, 55-98 (2011).
39. Degterev, A., et al. Chemical inhibitor of nonapoptotic cell death with therapeutic potential for ischemic brain injury. Nat Chem Biol 1, 112-119 (2005).
40. Fink, S. L., Bergsbaken, T. & Cookson, B. T. Anthrax lethal toxin and Salmonella elicit the common cell death pathway of caspase-1-dependent pyroptosis via distinct mechanisms. Proc Natl Acad Sci USA 105, 4312-4317 (2008).
41. Kranzusch, P. J., Lee, A. S., Berger, J. M. & Doudna, J. A. Structure of human cGAS reveals a conserved family of second-messenger enzymes in innate immunity. Cell Rep 3, 1362-1368 (2013).
42. Diner, E. J., et al. The innate immune DNA sensor cGAS produces a noncanonical cyclic dinucleotide that activates human STING. Cell Rep 3, 1355-1361 (2013).
43. Sun, L., Wu, J., Du, F., Chen, X. & Chen, Z. J. Cyclic GMP-AMP synthase is a cytosolic DNA sensor that activates the type I interferon pathway. Science 339, 786-791 (2013).
44. Schoggins, J. W., et al. A diverse range of gene products are effectors of the type I interferon antiviral response. Nature 472, 481-485 (2011).
45. Schoggins, J. W., et al. Pan-viral specificity of IFN-induced genes reveals new roles for cGAS in innate immunity. Nature 505, 691-695 (2014).
46. Zhang, X., et al. Cyclic GMP-AMP containing mixed phosphodiester linkages is an endogenous high-affinity ligand for STING. Mol Cell 51, 226-235 (2013).
47. Feher, J., et al. Mitochondrial alterations of retinal pigment epithelium in age-related macular degeneration. Neurobiol Aging 27, 983-993 (2006).
48. Terluk, M. R., et al. Investigating mitochondria as a target for treating age-related macular degeneration. J Neurosci 35, 7304-7311 (2015).
49. West, A. P., et al. Mitochondrial DNA stress primes the antiviral innate immune response. Nature 520, 553-557 (2015).
50. West, A. P., Shadel, G. S. & Ghosh, S. Mitochondria in innate immune responses. Nat Rev Immunol 11, 389-402 (2011).
51. Kroemer, G., Galluzzi, L. & Brenner, C. Mitochondrial membrane permeabilization in cell death. Physiol Rev 87, 99-163 (2007).
52. Galluzzi, L., Blomgren, K. & Kroemer, G. Mitochondrial membrane permeabilization in neuronal injury. Nat Rev Neurosci 10, 481-494 (2009).
53. Baines, C. P., et al. Loss of cyclophilin D reveals a critical role for mitochondrial permeability transition in cell death. Nature 434, 658-662 (2005).
54. Schinzel, A. C., et al. Cyclophilin D is a component of mitochondrial permeability transition and mediates neuronal cell death after focal cerebral ischemia. Proc Natl Acad Sci USA 102, 12005-12010 (2005).
55. Bonora, M., et al. Comprehensive analysis of mitochondrial permeability transition pore activity in living cells using fluorescence-imaging-based techniques. Nat Protoc 11, 1067-1080 (2016).
56. Miceli, M. V. & Jazwinski, S. M. Nuclear gene expression changes due to mitochondrial dysfunction in ARPE-19 cells: implications for age-related macular degeneration. Invest Ophthalmol Vis Sci 46, 1765-1773 (2005).
57. Grossniklaus, H. E., et al. Histopathologic and ultrastructural features of surgically excised subfoveal choroidal neovascular lesions: submacular surgery trials report no. 7. Arch Ophthalmol 123, 914-921 (2005).
58. Ding, X., Patel, M. & Chan, C. C. Molecular pathology of age-related macular degeneration. Prog Retin Eye Res 28, 1-18 (2009).
59. McLeod, D. S., et al. Distribution and Quantification of Choroidal Macrophages in Human Eyes With Age-Related Macular Degeneration. Invest Ophthalmol Vis Sci 57, 5843-5855 (2016).
60. Sakurai, E., Anand, A., Ambati, B. K., van Rooijen, N. & Ambati, J. Macrophage depletion inhibits experimental choroidal neovascularization. Invest Ophthalmol Vis Sci 44, 3578-3585 (2003).
61. Wang, X., et al. Requirement for Microglia for the Maintenance of Synaptic Function and Integrity in the Mature Retina. J Neurosci 36, 2827-2842 (2016).
62. Karijolich, J., Abernathy, E. & Glaunsinger, B. A. Infection-Induced Retrotransposon-Derived Noncoding RNAs Enhance Herpesviral Gene Expression via the N F-kappaB Pathway. PLoS Pathog 11, e1005260 (2015).
63. Panning, B. & Smiley, J. R. Activation of RNA polymerase III transcription of human Alu repetitive elements by adenovirus type 5: requirement for the E1b 58-kilodalton protein and the products of E4 open reading frames 3 and 6. Mol Cell Biol 13, 3231-3244 (1993).
64. Williams, W. P., Tamburic, L. & Astell, C. R. Increased levels of B1 and B2 SINE transcripts in mouse fibroblast cells due to minute virus of mice infection. Virology 327, 233-241 (2004).
65. Jang, K. L. & Latchman, D. S. HSV infection induces increased transcription of Alu repeated sequences by RNA polymerase III. FEBS Lett 258, 255-258 (1989).
66. Panning, B. & Smiley, J. R. Activation of expression of multiple subfamilies of human Alu elements by adenovirus type 5 and herpes simplex virus type 1. J Mol Biol 248, 513-524 (1995).
67. Nunnari, J. & Suomalainen, A. Mitochondria: in sickness and in health. Cell 148, 1145-1159 (2012).
68. Friedman, J. R. & Nunnari, J. Mitochondrial form and function. Nature 505, 335-343 (2014).
69. Fang, C., Wei, X. & Wei, Y. Mitochondrial DNA in the regulation of innate immune responses. Protein Cell 7, 11-16 (2016).
70. Shimada, K., et al. Oxidized mitochondrial DNA activates the NLRP3 inflammasome during apoptosis. Immunity 36, 401-414 (2012).
71. Nakahira, K., et al. Autophagy proteins regulate innate immune responses by inhibiting the release of mitochondrial DNA mediated by the NALP3 inflammasome. Nat Immunol 12, 222-230 (2011).
72. Zhang, Q., Itagaki, K. & Hauser, C. J. Mitochondrial DNA is released by shock and activates neutrophils via p38 map kinase. Shock 34, 55-59 (2010).
73. Zhang, Q., et al. Circulating mitochondrial DAMPs cause inflammatory responses to injury. Nature 464, 104-107 (2010).
74. Wei, X., et al. Cationic nanocarriers induce cell necrosis through impairment of Na(+)/K(+)-ATPase and cause subsequent inflammatory response. Cell Res 25, 237-253 (2015).
75. Yan, Y., et al. Dicer expression exhibits a tissue-specific diurnal pattern that is lost during aging and in diabetes. PLoS One 8, e80029 (2013).
76. Hung, T., et al. The Ro60 autoantigen binds endogenous retroelements and regulates inflammatory gene expression. Science 350, 455-459 (2015).
77. Hitomi, J., et al. Involvement of caspase-4 in endoplasmic reticulum stress-induced apoptosis and Abeta-induced cell death. J Cell Biol 165, 347-356 (2004).
78. Katayama, T., et al. Presenilin-1 mutations downregulate the signalling pathway of the unfolded-protein response. Nat Cell Biol 1, 479-485 (1999).
79. Ozcan, U., et al. Endoplasmic reticulum stress links obesity, insulin action, and type 2 diabetes. Science 306, 457-461 (2004).
80. Green, D. R. & Ferguson, T. A. The role of Fas ligand in immune privilege. Nat Rev Mol Cell Biol 2, 917-924 (2001).
81. Man, S. M. & Kanneganti, T. D. Converging roles of caspases in inflammasome activation, cell death and innate immunity. Nat Rev Immunol 16, 7-21 (2016).
82. Gurung, P., et al. FADD and caspase-8 mediate priming and activation of the canonical and noncanonical Nlrp3 inflammasomes. J Immunol 192, 1835-1846 (2014).
83. Yang, D., He, Y., Munoz-Planillo, R., Liu, Q. & Nunez, G. Caspase-11 Requires the Pannexin-1 Channel and the Purinergic P2X7 Pore to Mediate Pyroptosis and Endotoxic Shock. Immunity 43, 923-932 (2015).
84. Shaw, P. X., et al. Complement factor H genotypes impact risk of age-related macular degeneration by interaction with oxidized phospholipids. Proc Natl Acad Sci USA 109, 13757-13762 (2012).
85. Suzuki, M., et al. Chronic photo-oxidative stress and subsequent MCP-1 activation as causative factors for age-related macular degeneration. J Cell Sci 125, 2407-2415 (2012).
86. Suzuki, M., et al. Oxidized phospholipids in the macula increase with age and in eyes with age-related macular degeneration. Mol Vis 13, 772-778 (2007).
87. Marneros, A. G. NLRP3 inflammasome blockade inhibits VEGF-A-induced age-related macular degeneration. Cell Rep 4, 945-958 (2013).
88. Doyle, S. L., et al. NLRP3 has a protective role in age-related macular degeneration through the induction of IL-18 by drusen components. Nat Med 18, 791-798 (2012).
89. Doyle, S. L., et al. IL-18 attenuates experimental choroidal neovascularization as a potential therapy for wet age-related macular degeneration. Sci Transl Med 6, 230ra244 (2014).
90. Wallach, D., Kang, T. B., Dillon, C. P. & Green, D. R. Programmed necrosis in inflammation: Toward identification of the effector molecules. Science 352, aaf2154 (2016).
91. Hirano, Y., et al. IL-18 is not therapeutic for neovascular age-related macular degeneration. Nat Med 20, 1372-1375 (2014).
92. Wiesen, J. L. & Tomasi, T. B. Dicer is regulated by cellular stresses and interferons. Mol Immunol 46, 1222-1228 (2009).
93. van den Beucken, T., et al. Hypoxia promotes stem cell phenotypes and poor prognosis through epigenetic regulation of DICER. Nat Commun 5, 5203 (2014).
94. Ambati, J., Atkinson, J. P. & Gelfand, B. D. Immunology of age-related macular degeneration. Nat Rev Immunol 13, 438-451 (2013).
95. Ambati, J. & Fowler, B. J. Mechanisms of age-related macular degeneration. Neuron 75, 26-39 (2012).
96 Ambati, Method of inhibiting Alu RNA and therapeutic uses thereof (U.S. Pat. No. 8,809,517). Aug. 19, 2014.
97 Ambati and Fowler, Compositions and methods for treating retinal degradation (U.S. Pat. No. 9,326,983). May 3, 2016.
98 Ambati et al., Protection of cells from Alu-RNA-induced degeneration and inhibitors for protecting cells (U.S. Pat. No. 9,453,226). Sep. 27, 2016.
99 Ambati, Methods of inhibiting Alu RNA and therapeutic uses thereof (U.S. Pat. No. 9,464,289). Oct. 11, 2016.
100 Protection of cells from Alu-RNA-induced degeneration and inhibitors for protecting cells (Chinese Patent No. ZL 201280045438.0; International PCT/US12/46928). Nov. 9, 2016.
101 Ambati, Protection of cells from degeneration and treatment of geographic atrophy (U.S. Pat. No. 9,707,235). Jul. 18, 2017.

1. A method for preventing or treating age-related macular degeneration in a subject in need thereof by targeting at least one of the alternative, non-canonical inflammasome signaling molecules, protein complex, or signal transduction pathways in retinal pigment epithelium (RPE), the method comprising administering to the subject a pharmaceutical composition comprising a pharmaceutically-acceptable carrier, an effective amount of an inhibitor of noncanonical-inflammasome activation in RPE, and optionally an additional therapeutic agent, thereby preventing or treating age-related macular degeneration.

2. The method of claim 1, wherein said alternative, non-canonical inflammasome signaling molecule, protein complex, or signal transduction pathway is selected from the group consisting of cyclic GMP-AMP synthase (cGAS), Caspase-4, stimulator of interferon genes (STING), peptidyl-prolyl cis-trans isomerase F (PPIF), mitochondrial permeability transition pore (MPTP), Gasdermin D (GSDMD), interferon beta (IFN-β), and interferon-α/β receptor (IFNAR).

3. The method of claim 1, wherein said age-related macular degeneration is geographic atrophy.

4. The method of claim 1, wherein said inhibitor is selected from the group consisting of antisense oligonucleotide, small interfering RNA (siRNA), short hairpin RNA (shRNA), antibody, and biologically active fragments or homologs of said antibody.

5. The method of claim 4, wherein said antibody is selected from the group consisting of monoclonal antibody, humanized antibody, chimeric antibody, single chain antibody, and biologically active fragments and homologs thereof.

6. The method of claim 5, wherein said homolog comprises at least 95% sequence identity with said monoclonal antibody, humanized antibody, chimeric antibody, or single chain antibody.

7. The method of claim 1, wherein said inhibitor is selected from the group consisting of cGAS shRNA (shcGAS), cGAS siRNA, Caspase-4 shRNA, Caspase-4 siRNA, GSDMD shRNA, STING shRNA, PPIF shRNA, IFNB shRNA, IFN-β shRNA, IFNAR1 shRNA, and an IFN-β neutralizing antibody.

8. The method of claim 7, wherein said inhibitor is shcGAS or cGAS siRNA.

9-10. (canceled)

11. The method of claim 7, wherein said inhibitor is Caspase-4 shRNA or Caspase-4 siRNA.

12. The method of claim 11, wherein said Caspase-4 shRNA is SEQ ID NO: 16 and said Caspase-4 siRNA has a sequence selected from the group consisting of SEQ ID NOs: 9, 10, 11, 12, 13, and 14.

13. The method of claim 7, wherein said inhibitor is an IFN-β neutralizing antibody.

14. The method of claim 1, wherein said method protects RPE cells from cell death.

15. The method of claim 1, wherein said subject has been diagnosed with age-related macular degeneration or geographic atrophy.

16. The method of claim 1, wherein said subject is susceptible to age-related macular degeneration or geographic atrophy.

17. The method of claim 1, wherein two of said inhibitors are administered.

18. The method of claim 1, wherein said method inhibits Alu RNA induced RPE degeneration.

19. The method of claim 1, wherein an effective amount of an additional therapeutic agent is administered and said additional therapeutic agent is selected from the group consisting of cyclosporin A, Alu RNA antisense oligonucleotide, reverse transcriptase inhibitor, and IL-18 neutralizing antibody.

20. A method for preventing or inhibiting Alu RNA-induced retinal pigment epithelium (RPE) cell degeneration by targeting at least one of the alternative, non-canonical inflammasome signaling molecules or protein complex, the method comprising contacting said RPE cell with an inhibitor of at least one molecule or complex selected from the group consisting of cGAS, Caspase-4, STING, PPIF, MPTP, Gasdermin D, IFN-β, and IFNAR.

21-54. (canceled)

55. The method of claim 7, wherein said GSDMD shRNA has SEQ ID NO: 19, said STING shRNA has SEQ ID NO: 17, said PPIF shRNA has SEQ ID NO: 18, said IFN-β shRNA has SEQ ID NO: 20, and said IFNAR1 shRNA has SEQ ID NO: 21.

56-57. (canceled)


SEQUENCE LISTING (21 sequences)

SEQ ID NO: 1 - DNA, 59 bp, Homo sapiens
  source 1..59: mol_type = other DNA
ccggctttgataactgcgtgacatactcgagtatgtcacgcagttatcaaagttttttg

SEQ ID NO: 2 - DNA, 48 bp, Homo sapiens
  source 1..48: mol_type = other DNA
aagaagaaacatggcggctatccttctctcacatcgaaaaggaaattt

SEQ ID NO: 3 - protein, 522 aa, Homo sapiens
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 4 - DNA, 1802 bp, Homo sapiens
  source 1..1802: mol_type = other DNA
agcctggggttccccttcgggtcgcagactcttgtgtgcccgccagtagtgcttggtttccaacagctgctgctggctcttcctcttgcggccttttcctgaaacggattcttctttcggggaacagaaagcgccagccatgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaagatttctgcacctaatgaatttgatgtcatgtttaaactggaagtccccagaattcaactagaagaatattccaacactcgtgcatattactttgtgaaatttaaaagaaatccgaaagaaaatcctctgagtcagtttttagaaggtgaaatattatcagcttctaagatgctgtcaaagtttaggaaaatcattaaggaagaaattaacgacattaaagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaagaagaaacatggcggctatccttctctcacatcgaaaaggaaattttgaacaatcatggaaaatctaaaacgtgctgtgaaaacaaagaagagaaatgttgcaggaaagattgtttaaaactaatgaaataccttttagaacagctgaaagaaaggtttaaagacaaaaaacatctggataaattctcttcttatcatgtgaaaactgccttctttcacgtatgtacccagaaccctcaagacagtcagtgggaccgcaaagacctgggcctctgctttgataactgcgtgacatactttcttcagtgcctcaggacagaaaaacttgagaattattttattcctgaattcaatctattctctagcaacttaattgacaaaagaagtaaggaatttctgacaaagcaaattgaatatgaaagaaacaatgagtttccagtttttgatgaattttgagattgtatttttagaaagatctaagaactagagtcaccctaaatcctggagaatacaagaaaaatttgaaaaggggccagacgctgtggctcac

SEQ ID NO: 5 - protein, 377 aa, Homo sapiens
MAEGNHRKKPLKVLESLGKDFLTGVLDNLVEQNVLNWKEEEKKKYYDAKTEDKVRVMADSMQEKQRMAGQMLLQTFFNIDQISPNKKAHPNMEAGPPESGESTDALKLCPHEEFLRLCKERAEEIYPIKERNNRTRLALIICNTEFDHLPPRNGADFDITGMKELLEGLDYSVDVEENLTARDMESALRAFATRPEHKSSDSTFLVLMSHGILEGICGTVHDEKKPDVLLYDTIFQIFNNRNCLSLKDKPKVIIVQACRGANRGELWVRDSPASLEVASSQSSENLEEDAVYKTHVEKDFIAFCSSTPHNVSWRDSTMGSIFITQLITCFQKYSWCCHLEEVFRKVQQSFETPRAKAQMPTIERLSMTRYFYLFPGN

SEQ ID NO: 6 - DNA, 1319 bp, Homo sapiens
  source 1..1319: mol_type = other DNA
atacatagtttactttcatttttgactctgaggctctttccaacgctgtaaaaaaggacagaggctgttccctatggcagaaggcaaccacagaaaaaagccacttaaggtgttggaatccctgggcaaagatttcctcactggtgttttggataacttggtggaacaaaatgtactgaactggaaggaagaggaaaaaaagaaatattacgatgctaaaactgaagacaaagttcgggtcatggcagactctatgcaagagaagcaacgtatggcaggacaaatgcttcttcaaaccttttttaacatagaccaaatatcccccaataaaaaagctcatccgaatatggaggctggaccacctgagtcaggagaatctacagatgccctcaagctttgtcctcatgaagaattcctgagactatgtaaagaaagagctgaagagatctatccaataaaggagagaaacaaccgcacacgcctggctctcatcatatgcaatacagagtttgaccatctgcctccgaggaatggagctgactttgacatcacagggatgaaggagctacttgagggtctggactatagtgtagatgtagaagagaatctgacagccagggatatggagtcagcgctgagggcatttgctaccagaccagagcacaagtcctctgacagcacattcttggtactcatgtctcatggcatcctggagggaatctgcggaactgtgcatgatgagaaaaaaccagatgtgctgctttatgacaccatcttccagatattcaacaaccgcaactgcctcagtctgaaggacaaacccaaggtcatcattgtccaggcctgcagaggtgcaaaccgtggggaactgtgggtcagagactctccagcatccttggaagtggcctcttcacagtcatctgagaacctagaggaagatgctgtttacaagacccacgtggagaaggacttcattgctttctgctcttcaacgccacacaacgtgtcctggagagacagcacaatgggctctatcttcatcacacaactcatcacatgcttccagaaatattcttggtgctgccacctagaggaagtatttcggaaggtacagcaatcatttgaaactccaagggccaaagctcaaatgcccaccatagaacgactgtccatgacaagatatttctacctctttcctggcaattgaaaatggaagccacaagcagcccagccctccttaatcaacttcaaggagcaccttcattagtacagcttgcatatttaacattttgtatttcaataaaagtgaagacaaacga

SEQ ID NO: 7 - protein, 484 aa, Homo sapiens
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNFLTDGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPH

SEQ ID NO: 8 - DNA, 1823 bp, Homo sapiens
  source 1..1823: mol_type = other DNA
cctgggcgggccctgcgtcaggttgcagtttcacttttagctctgggcacctccagctcctgctcgccggacggctcccagggagagcagacgcgccagacgcgccaccctcggggcgccgacggtcacggagcatggggtcggcctttgagcgggtagtccggagagtggtccaggagctggaccatggtggggagttcatccctgtgaccagcctgcagagctccactggcttccagccctactgcctggtggttaggaagccctcaagctcatggttctggaaaccccgttataagtgtgtcaacctgtctatcaaggacatcctggagccggatgccgcggaaccagacgtgcagcgtggcaggagcttccacttctacgatgccatggatgggcagatacagggcagcgtggagctggcagccccaggacaggcaaagatcgcaggcggggccgcggtgtctgacagctccagcacctcaatgaatgtgtactcgctgagtgtggaccctaacacctggcagactctgctccatgagaggcacctgcggcagccagaacacaaagtcctgcagcagctgcgcagccgcggggacaacgtgtacgtggtgactgaggtgctacagacacagaaggaggtggaagtcacgcgcacccacaagcgggagggctcgggccggttttccctgcccggagccacgtgcttgcagggtgagggccagggccatctgagccagaagaagacggtcaccatcccctcaggcagcaccctcgcattccgggtggcccagctggttattgactctgacttggacgtccttctcttcccggataagaagcagaggaccttccagccacccgcgacaggccacaagcgttccacgagcgaaggcgcctggccacagctgccctctggcctctccatgatgaggtgcctccacaacttcctgacagatggggtccctgcggagggggcgttcactgaagacttccagggcctacgggcagaggtggagaccatctccaaggaactggagcttttggacagagagctgtgccagctgctgctggagggcctggagggggtgctgcgggaccagctggccctgcgagccttggaggaggcgctggagcagggccagagccttgggccggtggagcccctggacggtccagcaggtgctgtcctggagtgcctggtgttgtcctccggaatgctggtgccggaactcgctatccctgttgtctacctgctgggggcactgaccatgctgagtgaaacgcagcacaagctgctggcggaggcgctggagtcgcagaccctgttggggccgctcgagctggtgggcagcctcttggagcagagtgccccgtggcaggagcgcagcaccatgtccctgccccccgggctcctggggaacagctggggcgaaggagcaccggcctgggtcttgctggacgagtgtggcctagagctgggggaggacactccccacgtgtgctgggagccgcaggcccagggccgcatgtgtgcactctacgcctccctggcactgctatcaggactgagccaggagccccactagcctgtgcccgggcatggcctggcagctctccagcagggcagagtgtttgcccaccagctgctagccctaggaaggccaggagcccagtagccatgtggccagtctaccatggggcccaggagttggggaaacacaataaaggtggcatacgaaggaaaaaaaaaaaaaaaaaaaaaccaaaaaaaaaaaaaaaaaaaaaaaaaaaaaaaaaaaaaaaaaaaaaaaaaaaaaaaa

SEQ ID NO: 9 - DNA, 21 bp, Homo sapiens
  source 1..21: mol_type = other DNA
gtgtagatgtagaagagaatt

SEQ ID NO: 10 - DNA, 21 bp, Homo sapiens
  source 1..21: mol_type = other DNA
cctagaggaagatgctgtttt

SEQ ID NO: 11 - DNA, 19 bp, Homo sapiens
  source 1..19: mol_type = other DNA
ctacactgtggttgacgaa

SEQ ID NO: 12 - RNA, 19 nt, Homo sapiens
  source 1..19: mol_type = other RNA
ccatagaacgagcaacctt

SEQ ID NO: 13 - RNA, 19 nt, Homo sapiens
  source 1..19: mol_type = other RNA
cagcagaatctacaaatat

SEQ ID NO: 14 - RNA, 19 nt, Homo sapiens
  source 1..19: mol_type = other RNA
cggatgtgctgctttatga

SEQ ID NO: 15 - DNA, 59 bp, Homo sapiens
  source 1..59: mol_type = other DNA
ccggcctgctgtaacacttcttattctcgagaataagaagtgttacagcaggttttttg

SEQ ID NO: 16 - DNA, 57 bp, Homo sapiens
  source 1..57: mol_type = other DNA
ccggagactatgtaaagaaagagctctcgagagctctttctttacatagtctttttt

SEQ ID NO: 17 - DNA, 59 bp, Homo sapiens
  source 1..59: mol_type = other DNA
ccggccaacattcgcttcctggatactcgagtatccaggaagcgaatgttggttttttg

SEQ ID NO: 18 - DNA, 58 bp, Homo sapiens
  source 1..58: mol_type = other DNA
ccggctgtggccagttgagctaatcctcgaggattagctcaactggccacagtttttg

SEQ ID NO: 19 - DNA, 59 bp, Homo sapiens
  source 1..59: mol_type = other DNA
ccggcaacctgtctatcaaggacatctcgagatgtccttgatagacaggttgttttttg

SEQ ID NO: 20 - DNA, 57 bp, Homo sapiens
  source 1..57: mol_type = other DNA
ccggcagagtggaaatcctaaggaactcgagttccttaggatttccactctgttttt

SEQ ID NO: 21 - DNA, 58 bp, Homo sapiens
  source 1..58: mol_type = other DNA
ccgggccaagattcaggaaattattctcgagaataatttcctgaatcttggctttttg